(19) United States
(12) Patent Application Publication
(10) Pub. No.: US 2023/0183639 A1
(43) Pub. Date: Jun. 15, 2023

(54) IMPROVED MICROORGANISMS FOR ARABINOSE FERMENTATION

(71) Applicant: Novozymes A/S, Bagsvaerd (DK)

(72) Inventor: Monica Tassone, West Sacramento, CA (US)

(73) Assignee: Novozymes A/S, Bagsvaerd (DK)

(21) Appl. No.: 17/924,917

(22) PCT Filed: May 12, 2021
(86) PCT No.: PCT/US2021/032054

§ 371 Date: Nov. 11, 2022

Related U.S. Application Data
(60) Provisional application No. 63/024,010, filed on May 13, 2020.

(51) Int. Cl. C12N 1/18; C12N 15/52

(52) U.S. Cl. CPC C12N 1/185; C12N 15/52

(57) ABSTRACT

Described herein are recombinant host organisms having an active arabinose fermentation pathway and further comprising a heterologous polynucleotide a heterologous polynucleotide encoding a L-xylulose reductase (LXR). Also described are processes for 5 producing a fermentation product, such as ethanol, from starch or cellulosic-containing material with the recombinant host organisms.

### REFERENCE TO A SEQUENCE LISTING

This application contains a Sequence Listing in computer readable form, which is incorporated herein by reference.

### BACKGROUND

Production of ethanol from starch and cellulosic containing materials is well-known in the art.

The most commonly industrially used commercial process for starch-containing material, often referred to as a “conventional process”, includes liquefying gelatinized starch at high temperature (about 85° C.) using typically a bacterial alpha-amylase, followed by simultaneous saccharification and fermentation (SSF) carried out anaerobically in the presence of typically a glucoamylase and a Saccharomyces cerevisiae yeast.

Yeasts which are used for production of ethanol for use as fuel, such as in the corn ethanol industry, require several characteristics to ensure cost effective production of the ethanol. These characteristics include ethanol tolerance, low by-product yield, rapid fermentation, and the ability to limit the amount of residual sugars remaining in the ferment. Such characteristics have a marked effect on the viability of the industrial process.

Yeast of the genus Saccharomyces exhibits many of the characteristics required for production of ethanol. In particular, strains of Saccharomyces cerevisiae are widely used for the production of ethanol in the fuel ethanol industry. Strains of Saccharomyces cerevisiae that are widely used in the fuel ethanol industry have the ability to produce high yields of ethanol under fermentation conditions found in, for example, the fermentation of corn mash. An example of such a strain is the yeast used in commercially available ethanol yeast product called ETHANOL RED®.

Efforts to improve arabinose utilization by recombinant techniques to modify Saccharomyces yeast have been described in e.g., WO2002/066616, U.S. Pat. Nos. 9,551,015 and 7,977,083. However, there remains a need for improved arabinose sugar utilization in genetically-engineered yeast for production of bioethanol in an economically and commercially relevant scale.

### SUMMARY

Described herein are, inter alia, methods for producing a fermentation product, such as ethanol, from starch or cellulosic-containing material, and microorganisms suitable for use in such processes. The Applicant surprisingly found a cluster of related L-xylulose reductase (LXR) genes that, when expressed in a yeast host, can provide exceptional arabinose utilization. These related genes are structurally distinct from those LXRs used in the art, as described in more detail below, which the Applicant believes is responsible for their unexpected superior performance. The Applicant also found that yeast can utilize arabinose in a fungal arabinose pathway that expresses the described heterologous LXR gene together with a heterologous aldose reductase (AR) and L-arabinitol 4-dehydrogenase (LAD), but without the need for expressing other heterologous genes of the pathway (See FIG. 1).

A first aspect relates to a recombinant host cell having an active arabinose fermentation pathway, wherein the cell comprises:

(i) a heterologous polynucleotide encoding an aldose reductase (AR),

(ii) a heterologous polynucleotide encoding a L-arabinitol 4-dehydrogenase (LAD), and

(iii) a heterologous polynucleotide encoding a L-xylulose reductase (LXR);

wherein the L-xylulose reductase (LXR) has a mature polypeptide sequence with at least 60%, e.g., at least 65%, 70%, 75%, 80%, 85%, 90%, 95%, 97%, 98%, 99%, or 100% sequence identity to any one of SEQ ID NOs: 297, 300, 302 and 304.

In one embodiment, the L-xylulose reductase (LXR) differs by no more than ten amino acids, e.g., by no more than five amino acids, by no more than four amino acids, by no more than three amino acids, by no more than two amino acids, or by one amino acid from the amino acid sequence of any one of any one of SEQ ID NOs: 297, 300, 302 and 304. In one embodiment, the LXR comprises or consists of the amino acid sequence of any one of SEQ ID NOs: 297, 300, 302 and 304.

In one embodiment, the aldose reductase (AR) has a mature polypeptide sequence with at least 60%, e.g., at least 65%, 70%, 75%, 80%, 85%, 90%, 95%, 97%, 98%, 99%, or 100% sequence identity to any one of SEQ ID NOs: 281-285. In one embodiment, the AR differs by no more than ten amino acids, e.g., by no more than five amino acids, by no more than four amino acids, by no more than three amino acids, by no more than two amino acids, or by one amino acid from the amino acid sequence of any one of any one of SEQ ID NOs: 281-285. In one embodiment, the AR comprises or consists of the amino acid sequence of any one of SEQ ID NOs: 281-285.

In one embodiment, the L-arabinitol 4-dehydrogenase (LAD) has a mature polypeptide sequence with at least 60%, e.g., at least 65%, 70%, 75%, 80%, 85%, 90%, 95%, 97%, 98%, 99%, or 100% sequence identity to any one of SEQ ID NOs: 286-296. In one embodiment, the LAD differs by no more than ten amino acids, e.g., by no more than five amino acids, by no more than four amino acids, by no more than three amino acids, by no more than two amino acids, or by one amino acid from the amino acid sequence of any one of any one of SEQ ID NOs: 286-296. In one embodiment, the LAD comprises or consists of the amino acid sequence of any one of SEQ ID NOs: 286-296.

In some embodiments, the cell further comprises one or more heterologous polynucleotides selected from: (iv) a heterologous polynucleotide encoding a xylitol dehydrogenase (XDH); and (v) a heterologous polynucleotide encoding a xylulokinase (XK).

In one embodiment, the xylitol dehydrogenase (XDH) has a mature polypeptide sequence with at least 60%, e.g., at least 65%, 70%, 75%, 80%, 85%, 90%, 95%, 97%, 98%, 99%, or 100% sequence identity to SEQ ID NO: 309. In one embodiment, the XDH differs by no more than ten amino acids, e.g., by no more than five amino acids, by no more than four amino acids, by no more than three amino acids, by no more than two amino acids, or by one amino acid from the amino acid sequence of SEQ ID NO: 309. In one embodiment, the XDH comprises or consists of the amino acid sequence of SEQ ID NO: 309.

In one embodiment, the xylulokinase (XK) has a mature polypeptide sequence with at least 60%, e.g., at least 65%, 70%, 75%, 80%, 85%, 90%, 95%, 97%, 98%, 99%, or 100% sequence identity to SEQ ID NO: 75, SEQ ID NO: 310 or SEQ ID NO: 311. In one embodiment, the XK differs by no more than ten amino acids, e.g., by no more than five amino acids, by no more than four amino acids, by no more than three amino acids, by no more than two amino acids, or by one amino acid from the amino acid sequence of SEQ ID NO: 75, SEQ ID NO: 310 or SEQ ID NO: 311. In one embodiment, the XK comprises or consists of the amino acid sequence of SEQ ID NO: 75, SEQ ID NO: 310 or SEQ ID NO: 311.

In one embodiment, the heterologous polynucleotide of (i), (ii), (iii), (iv) and/or (v) is operably linked to a promoter that is foreign to the polynucleotide.

In one embodiment, the cell further comprises heterologous polynucleotide encoding a non-phosphorylating NADP-dependent glyceraldehyde-3-phosphate dehydrogenase (GAPN). In one embodiment, the GAPN has a mature polypeptide sequence with at least 60%, e.g., at least 65%, 70%, 75%, 80%, 85%, 90%, 95%, 97%, 98%, 99%, or 100% sequence identity to any one of SEQ ID NOs: 262-280. In one embodiment, the GAPN differs by no more than ten amino acids, e.g., by no more than five amino acids, by no more than four amino acids, by no more than three amino acids, by no more than two amino acids, or by one amino acid from the amino acid sequence of any one of SEQ ID NOs: 262-280. In one embodiment, the GAPN comprises or consists of the amino acid sequence of any one of SEQ ID NOs: 262-280. In one embodiment, the heterologous polynucleotide encoding the GAPN is operably linked to a promoter that is foreign to the polynucleotide.

In one embodiment, the recombinant host cell comprises an active xylose fermentation pathway. In one embodiment, the cell comprises one or more active xylose fermentation pathway genes selected from: a heterologous polynucleotide encoding a xylose isomerase (XI), and a heterologous polynucleotide encoding a xylulokinase (XK). In one embodiment, the cell comprises one or more active xylose fermentation pathway genes selected from: a heterologous polynucleotide encoding an aldose reductase (AR), a heterologous polynucleotide encoding a xylitol dehydrogenase (XDH), and a heterologous polynucleotide encoding a xylulokinase (XK).

In one embodiment, the recombinant host cell further comprises a heterologous polynucleotide encoding a glucoamylase. In one embodiment, the glucoamylase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 8,102-113,229,230 and 244-250. In one embodiment, the heterologous polynucleotide encoding the glucoamylase is operably linked to a promoter that is foreign to the polynucleotide.

In one embodiment, the recombinant host cell further comprises a heterologous polynucleotide encoding an alpha-amylase. In one embodiment, the alpha-amylase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 76-101, 121-174, 231 and 251-256. In one embodiment, the heterologous polynucleotide encoding the alpha-amylase is operably linked to a promoter that is foreign to the polynucleotide.

In one embodiment, the recombinant host cell further comprises a heterologous polynucleotide encoding a phospholipase. In one embodiment, the phospholipase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 235, 236, 237, 238, 239, 240, 241 and 242. In one embodiment, the heterologous polynucleotide encoding phospholipase is operably linked to a promoter that is foreign to the polynucleotide.

In one embodiment, the recombinant host cell further comprises a heterologous polynucleotide encoding a trehalase. In one embodiment, the trehalase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 175-226. In one embodiment, the heterologous polynucleotide encoding the trehalase is operably linked to a promoter that is foreign to the polynucleotide.

In one embodiment, the recombinant host cell further comprises a heterologous polynucleotide encoding a protease. In one embodiment, the protease has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 9-73. In one embodiment, the heterologous polynucleotide encoding the protease is operably linked to a promoter that is foreign to the polynucleotide.

In one embodiment, the recombinant host cell further comprises a heterologous polynucleotide encoding a pullulanase. In one embodiment, the pullulanase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 114-120. In one embodiment, the heterologous polynucleotide encoding the pullulanase is operably linked to a promoter that is foreign to the polynucleotide.

In one embodiment, the recombinant host cell is capable of higher anaerobic growth rate on pentose (e.g., xylose and/or arabinose) compared to the same cell without the heterologous polynucleotide encoding a L-xylulose reductase (LXR) (e.g., under conditions described in Example 2). In one embodiment, the cell is capable of higher anaerobic growth rate on pentose (e.g., xylose and/or arabinose) compared to the same cell encoding the L-xylulose reductase (LXR) of SEQ ID NO: 305 (e.g., under conditions described in Example 2).

In one embodiment, the recombinant host cell is capable of a higher rate of pentose consumption (e.g., at least 5%, 10%, 15%, 20%, 25%, 30%, 35%, 40%, 45%, 50%, 60%, 75% or 90% higher xylose and/or arabinose consumption) compared to the same cell without the heterologous polynucleotide encoding a L-xylulose reductase (LXR) (e.g., under conditions described in Example 2). In one embodiment, the recombinant host cell is capable of a higher rate of pentose consumption (e.g., at least 5%, 10%, 15%, 20%, 25%, 30%, 35%, 40%, 45%, 50%, 60%, 75% or 90% higher xylose and/or arabinose consumption) compared to the same cell encoding the L-xylulose reductase (LXR) of SEQ ID NO: 305 (e.g., under conditions described in Example 2).

In one embodiment, the recombinant host cell is capable of higher pentose consumption (e.g., xylose and/or arabinose) compared to the same cell without the heterologous polynucleotide encoding a L-xylulose reductase (LXR) at about or after 120 hours fermentation (e.g., under conditions described in Example 2). In one embodiment, the recombinant host cell is capable of higher pentose consumption (e.g., xylose and/or arabinose) compared to the same cell encoding the L-xylulose reductase (LXR) of SEQ ID NO: 305 at about or after 120 hours fermentation (e.g., under conditions described in Example 2). In one embodiment, the recombinant host cell is capable of consuming more than 65%, e.g., at least 70%, 75%, 80%, 85%, 90%, 95% of pentose (e.g., xylose and/or arabinose) in the medium at about or after 120 hours fermentation (e.g., under conditions described in Example 2).

In one embodiment, the recombinant host cell is capable of higher ethanol production compared to the same cell without the heterologous polynucleotide of (i), (ii), (iii), (iv) and/or (v) under the same conditions (e.g., after 40 hours of fermentation). In one embodiment, the recombinant host cell is capable of higher ethanol production compared to the same cell encoding the L-xylulose reductase (LXR) of SEQ ID NO: 305 under the same conditions (e.g., after 40 hours of fermentation).

In one embodiment, the recombinant host cell further comprises a heterologous polynucleotide encoding a transketolase (TKL1). In one embodiment, the cell further comprises a heterologous polynucleotide encoding a transaldolase (TAL1).

In one embodiment, the recombinant host cell further comprises a disruption (e.g., inactivation) to an endogenous gene encoding a glycerol 3-phosphate dehydrogenase (GPD). In one embodiment, the cell further comprises a disruption (e.g., inactivation) to an endogenous gene encoding a glycerol 3-phosphatase (GPP). In one embodiment, the cell produces a decreased amount of glycerol (e.g., at least 25% less, at least 50% less, at least 60% less, at least 70% less, at least 80% less, or at least 90% less) compared to the cell without the disruption to the endogenous gene encoding the GPD and/or GPP when cultivated under identical conditions.

In one embodiment, the recombinant host cell is a yeast cell. In one embodiment, the cell is a Saccharomyces, Rhodotorula, Schizosaccharomyces, Kluyveromyces, Pichia, Hansenula, Rhodosporidium, Candida, Yarrowia, Lipomyces, Cryptococcus, or Dekkera sp. yeast cell. In one embodiment, the cell is Saccharomyces cerevisiae.

A second aspect relates to methods of producing a fermentation product from a starch-containing or cellulosic-containing material, the method comprising:

(a) saccharifying the starch-containing or cellulosic-containing material; and

(b) fermenting the saccharified material of step (a) with the recombinant host cell of the first aspect.

In one embodiment, the method comprises liquefying the starch-containing material at a temperature above the initial gelatinization temperature in the presence of an alpha-amylase and/or a protease prior to saccharification. In one embodiment, the fermentation product is ethanol.

A third aspect relates to methods of producing a derivative of host cell of the first aspect, comprising culturing a host cell of the first aspect with a second host cell under conditions which permit combining of DNA between the first and second host cells, and screening or selecting for a derived host cell.

A fourth aspect relates to compositions comprising the host cell of the first aspect with one or more naturally occurring and/or non-naturally occurring components, such as components selected from the group consisting of: surfactants, emulsifiers, gums, swelling agents, and antioxidants.

### BRIEF DESCRIPTION OF THE FIGURES

FIG. 1 shows arabinose fermentation pathways from L-arabinose to D-xylulose 5-phosphate, which is then fermented to ethanol via the pentose phosphate pathway. The bacterial pathway utilizes genes L-arabinose isomerase (AI), L-ribulokinase (RK), and L-ribulose-5-P4-epimerase (R5PE) to convert L-arabinose to D-xylulose 5-phosphate. The fungal pathway proceeds using aldose reductase (AR), L-arabinitol 4-dehydrogenase (LAD), L-xylulose reductase (LXR), xylitol dehydrogenase (XDH) and xylulokinase (XK).

FIG. 2 shows a functional diversity tree for the expressed heterologous L-xylulose reductases (LXRs). The cluster of LXRs that provided substantial cell growth on arabinose agar plates is indicated with a box. The remaining LXRs provided minimal or no cell growth under the same conditions, including the previously described T. reesei LXR of SEQ ID NO: 305 (shown with an asterisk).

FIG. 3 shows a Multiple Sequence Alighnment of the L-xylulose reductases (LXRs) of SEQ ID NOs: 297, 300, 302 and 304.

FIG. 4 shows xylose fermentation pathways from D-xylose to D-xylulose 5-phosphate, which is then fermented to ethanol via the pentose phosphate pathway. The oxido-reductase pathway uses an aldolase reductase (AR, such as xylose reductase (XR)) to reduce D-xylose to xylitol followed by oxidation of xylitol to D-xylulose with xylitol dehydrogenase (XDH). The isomerase pathway uses xylose isomerase (XI) to convert D-xylose directly into D-xylulose. D-xylulose is then converted to D-xylulose-5-phosphate with xylulokinase (XK).

FIG. 5 shows a plasmid map of pMLBA635.

FIG. 6 shows a plasmid map of pMLBA638.

### DEFINITIONS

Unless defined otherwise or clearly indicated by context, all technical and scientific terms used herein have the same meaning as commonly understood by one of ordinary skill in the art.

Aldose reductase: The term “aldose reductase” or “AR” is classified as E.C. 1.1.1.21 and means an enzyme that catalyzes the conversion of L-arabinose to L-arabitol. Some aldose reductase genes may be unspecific and have activity on D-xylose to produce xylitol (AKA, D-xylose reductase; classified as E.C. 1.1.1.307). Aldose reductase activity can be determined using methods known in the art (e.g., Kuhn, et al., 1995, Appl. Environ. Microbiol. 61 (4), 1580-1585).

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-amylase: The term “alpha amylase” means an 1,4-alpha-D-glucan glucanohydrolase, EC. 3.2.1.1, which catalyze hydrolysis of starch and other linear and branched 1,4-glucosidic oligo- and polysaccharides. Alpha-amylase activity can be determined using methods known in the art (e.g., using an alpha amylase assay described WO02020/023411).

L-arabinitol dehydrogenase: The term “L-arabinitol dehydrogenase” or “LAD” is classified as E.C. 1.1.1.12 and means an enzyme that catalyzes the conversion of L-arabitol to L-xylulose. L-arabinitol dehydrogenase activity can be determined using methods known in the art (e.g., as described in U.S. Pat. No. 7,527,951).

Auxiliary Activity 9: The term “Auxiliary Activity 9” or “AA9” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic-containing material by an enzyme having cellulolytic activity. Cellulolytic enhancing activity can be determined by measuring the increase in reducing sugars or the increase of the total of cellobiose and glucose from the hydrolysis of a cellulosic-containing material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40 C-80° C., e.g., 50° C., 55° C., 60° C., 65° C., or 70° C., and a suitable pH, such as 4-9, e.g., 4.5, 5.0, 5.5, 6.0, 6.5, 7.0, 7.5, 8.0, or 8.5, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST® 1.5 L (Novozymes A/S, Bagsværd, Denmark) and beta-glucosidase as the source of the cellulolytic activity, wherein the beta-glucosidase is present at a weight of at least 2-5% protein of the cellulase protein loading. In one embodiment, the beta-glucosidase is an Aspergillus oryzae beta-glucosidase (e.g., recombinantly produced in Aspergillus oryzae according to WO 02/095014). In another embodiment, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (4-(1,1,3,3-tetramethylbutyl)phenyl-polyethylene glycol) for 24-96 hours at 40° C. followed by determination of the glucose released from the PASC.

AA9 polypeptide enhancing activity can also be determined according to WO02013/028928 for high temperature compositions.

AA9 polypeptides enhance the hydrolysis of a cellulosic-containing material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN®20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN®20.

Catalase: The term “catalase” means a hydrogen-peroxide:hydrogen-peroxide oxidoreductase (EC 1.11.1.6) that catalyzes the conversion of 2H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Catalytic domain: The term “catalytic domain” means the region of an enzyme containing the catalytic machinery of the enzyme.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase 1) or non-reducing end (cellobiohydrolase 11) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581.

Cellulolytic enzyme or cellulase: The term “cellulolytic enzyme” or “cellulase” means one or more (e.g., several) enzymes that hydrolyze a cellulosic-containing material. Such enzymes include endoglucanase(s), cellobiohydrolase(s), beta-glucosidase(s), or combinations thereof. The two basic approaches for measuring cellulolytic enzyme activity include: (1) measuring the total cellulolytic enzyme activity, and (2) measuring the individual cellulolytic enzyme activities (endoglucanases, cellobiohydrolases, and beta-glucosidases) as reviewed in Zhang et al., 2006, Biotechnology Advances 24: 452-481. Total cellulolytic enzyme activity can be measured using insoluble substrates, including Whatman N1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59:257-68).

Cellulolytic enzyme activity can be determined by measuring the increase in production/release of sugars during hydrolysis of a cellulosic-containing material by cellulolytic enzyme(s) under the following conditions: 1-50 mg of cellulolytic enzyme protein/g of cellulose in pretreated corn stover (PCS) (or other pretreated cellulosic-containing material) for 3-7 days at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0, compared to a control hydrolysis without addition of cellulolytic enzyme protein. Typical conditions are 1 ml reactions, washed or unwashed PCS, 5% insoluble solids (dry weight), 50 mM sodium acetate pH 5, 1 mM MnSO₄, 50° C., 55° C., or 60° C., 72 hours, sugar analysis by AMINEX® HPX-87H column chromatography (Bio-Rad Laboratories, Inc., Hercules, Calif., USA).

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using from cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)).

Endogenous gene: The term “endogenous gene” means a gene that is native to the referenced host cell. “Endogenous gene expression” means expression of an endogenous gene.

Endoglucanase: The term “endoglucanase” means a 4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59:257-268, at pH 5, 40° C.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences that provide for its expression.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as ethanol. In some instances, the fermentation medium is derived from a natural source, such as sugar cane, starch, or cellulose, and may be the result of pretreating the source by enzymatic hydrolysis (saccharification). The term fermentation medium is understood herein to refer to a medium before the fermenting organism is added, such as, a medium resulting from a saccharification process, as well as a medium used in a simultaneous saccharification and fermentation process (SSF).

Glucoamylase: The term “glucoamylase” (1,4-alpha-D-glucan glucohydrolase, EC 3.2.1.3) is defined as an enzyme that catalyzes the release of D-glucose from the non-reducing ends of starch or related oligo- and polysaccharide molecules. For purposes of the present invention, glucoamylase activity may be determined according to the procedures known in the art, such as those described in WO02020/023411.

Hemicellulolytic enzyme or hemicellulase: The term “hemicellulolytic enzyme” or “hemicellulase” means one or more (e.g., several) enzymes that hydrolyze a hemicellulosic material. See, for example, Shallom and Shoham, 2003, Current Opinion In Microbiology 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates for these enzymes, hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature such as 40° C.-80° C., e.g., 50° C., 55° C., 60° C., 65° C., or 70° C., and a suitable pH such as 4-9, e.g., 5.0, 5.5, 6.0, 6.5, or 7.0.

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

Host cell: The term “host cell” means any cell type that is susceptible to transformation, transfection, transduction, and the like with a nucleic acid construct or expression vector comprising a polynucleotide described herein. The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Mature polypeptide: The term “mature polypeptide” is defined herein as a polypeptide having biological activity that is in its final form following translation and any post-translational modifications, such as N-terminal processing, C-terminal truncation, glycosylation, phosphorylation, etc. The mature polypeptide sequence lacks a signal sequence, which may be determined using techniques known in the art (See, e.g., Zhang and Henzel, 2004, Protein Science 13: 2819-2824). The term “mature polypeptide coding sequence” means a polynucleotide that encodes a mature polypeptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

Non-phosphorylating NADP-dependent glyceraldehyde-3-phosphate dehydrogenase (GAPN): The term “non-phosphorylating NADP-dependent glyceraldehyde-3-phosphate dehydrogenase”, “NADP-dependent glyceraldehyde-3-phosphate dehydrogenase” or “GAPN” is defined herein as an enzyme that catalyzes the chemical reaction of glyceraldehyde-3-phosphate and NADP+ to 3-phosphoglycerate and NADPH (e.g., EC 1.2.1.9). GAPN activity may be determined from cell-free extracts as described in the art, e.g., as described in Tamoi et al., 1996, Biochem. J. 316, 685-690. For example, GAPN activity may be measured spectrophotometrically by monitoring the absorbance change following NADPH oxidation at 340 nm in a reaction mixture containing 100 mM Tris/HCl buffer (pH 8.0), 10 mM MgCl₂, 10 mM GSH, 5 mM ATP, 0.2 mM NADPH, 2 units of 3-phosphoglyceric phosphokinase, 2 mM 3-phosphoglyceric acid and the enzyme.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Pentose: The term “pentose” means a five-carbon monosaccharide (e.g., xylose, arabinose, ribose, lyxose, ribulose, and xylulose). Pentoses, such as D-xylose and L-arabinose, may be derived, e.g., through saccharification of a plant cell wall polysaccharide.

Active pentose fermentation pathway: As used herein, a host cell or fermenting organism having an “active pentose fermentation pathway” produces active enzymes necessary to catalyze each reaction of a metabolic pathway in a sufficient amount to produce a fermentation product (e.g., ethanol) from pentose, and therefore is capable of producing the fermentation product in measurable yields when cultivated under fermentation conditions in the presence of pentose. A host cell or fermenting organism having an active pentose fermentation pathway comprises one or more active pentose fermentation pathway genes. A “pentose fermentation pathway gene” as used herein refers to a gene that encodes an enzyme involved in an active pentose fermentation pathway. In some embodiments, the active pentose fermentation pathway is an “active xylose fermentation pathway” (ie produces a fermentation product, such as ethanol, from xylose) or an “active arabinose fermentation pathway (ie produces a fermentation product, such as ethanol, from arabinose).

The active enzymes necessary to catalyze each reaction in an active pentose fermentation pathway may result from activities of endogenous gene expression, activities of heterologous gene expression, or from a combination of activities of endogenous and heterologous gene expression, as described in more detail herein.

Phospholipase: The term “phospholipase” means an enzyme that catalyzes the conversion of phospholipids into fatty acids and other lipophilic substances, such as phospholipase A (EC numbers 3.1.1.4, 3.1.1.5 and 3.1.1.32) or phospholipase C (EC numbers 3.1.4.3 and 3.1.4.11). Phospholipase activity may be determined using activity assays known in the art.

Pretreated corn stover: The term “Pretreated Corn Stover” or “PCS” means a cellulosic-containing material derived from corn stover by treatment with heat and dilute sulfuric acid, alkaline pretreatment, neutral pretreatment, or any pretreatment known in the art.

Protease: The term “protease” is defined herein as an enzyme that hydrolyses peptide bonds. It includes any enzyme belonging to the EC 3.4 enzyme group (including each of the thirteen subclasses thereof). The EC number refers to Enzyme Nomenclature 1992 from NC-IUBMB, Academic Press, San Diego, Calif., including supplements 1-5 published in Eur. J. Biochem. 223: 1-5 (1994); Eur. J. Biochem. 232: 1-6 (1995); Eur. J. Biochem. 237: 1-5 (1996); Eur. J. Biochem. 250: 1-6 (1997); and Eur. J. Biochem. 264: 610-650 (1999); respectively. The term “subtilases” refer to a sub-group of serine protease according to Siezen et al., 1991, Protein Engng. 4: 719-737 and Siezen et al., 1997, Protein Science 6: 501-523. Serine proteases or serine peptidases is a subgroup of proteases characterised by having a serine in the active site, which forms a covalent adduct with the substrate. Further the subtilases (and the serine proteases) are characterised by having two active site amino acid residues apart from the serine, namely a histidine and an aspartic acid residue. The subtilases may be divided into 6 sub-divisions, i.e. the Subtilisin family, the Thermitase family, the Proteinase K family, the Lantibiotic peptidase family, the Kexin family and the Pyrolysin family. The term “protease activity” means a proteolytic activity (EC 3.4). Protease activity may be determined using methods described in the art (e.g., US 2015/0125925) or using commercially available assay kits (e.g., Sigma-Aldrich).

Pullulanase: The term “pullulanase” means a starch debranching enzyme having pullulan 6-glucano-hydrolase activity (EC 3.2.1.41) that catalyzes the hydrolysis the α-1,6-glycosidic bonds in pullulan, releasing maltotriose with reducing carbohydrate ends. For purposes of the present invention, pullulanase activity can be determined according to a PHADEBAS assay or the sweet potato starch assay described in WO2016/087237.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Referenced Sequence−Total Number of Gaps in Alignment)

Signal peptide: The term “signal peptide” is defined herein as a peptide linked (fused) in frame to the amino terminus of a polypeptide having biological activity and directs the polypeptide into the cell's secretory pathway. Signal sequences may be determined using techniques known in the art (See, e.g., Zhang and Henzel, 2004, Protein Science 13: 2819-2824).

Trehalase: The term “trehalase” means an enzyme which degrades trehalose into its unit monosaccharides (i.e., glucose). Trehalases are classified in EC 3.2.1.28 (alpha,alpha-trehalase) and EC. 3.2.1.93 (alpha,alpha-phosphotrehalase). The EC classes are based on recommendations of the Nomenclature Committee of the International Union of Biochemistry and Molecular Biology (IUBMB). Description of EC classes can be found on the internet, e.g., on “http://www.expasy.org/enzyme/”. Trehalases are enzymes that catalyze the following reactions:

EC 3.2.1.28: Alpha,alpha-trehalose+H₂O [symbol] 2 D-glucose;

EC 3.2.1. 93: Alpha,alpha-trehalose 6-phosphate+H₂O [symbol] D-glucose+D-glucose 6-phosphate.

Trehalase activity may be determined according to procedures known in the art.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity can be determined with 0.2% AZCL-arabinoxylan as substrate in 0.01% TRITON® X-100 and 200 mM sodium phosphate pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

Xylitol dehydrogenase: The term “xylitol dehydrogenase” or “XDH” (AKA D-xylulose reductase) is classified as E.C. 1.1.1.9 and means an enzyme that catalyzes the conversion of xylitol to D-xylulose. Xylitol dehydrogenase activity can be determined using methods known in the art (e.g., Richard et al., 1999, FEBS Letters 457, 135-138).

Xylose isomerase: The term “xylose isomerase” or “XI” means an enzyme which can catalyze D-xylose into D-xylulose in vivo, and convert D-glucose into D-fructose in vitro. Xylose isomerase is also known as “glucose isomerase” and is classified as E.C. 5.3.1.5. As the structure of the enzyme is very stable, the xylose isomerase is a good model for studying the relationships between protein structure and functions (Karimaki et al., Protein Eng Des Sel, 12004, 17 (12):861-869). Xylose Isomerase activity may be determined using techniques known in the art (e.g., a coupled enzyme assay using D-sorbitol dehygrogenase, as described by Verhoeven et al., 2017, Sci Rep 7, 46155).

Xylulokinase: The term “xylulokinase” or “XK” is classified as E.C. 2.7.1.17 and means an enzyme that catalyzes the conversion of D-xylulose to D-xylulose 5-phosphate. Xylulokinase activity can be determined using methods known in the art (e.g., Richard et al., 2000, FEBS Microbiol. Letters 190, 39-43)

L-xylulose reductase: The term “L-xylulose reductase” or “LXR” is classified as E.D. 1.1.1.10 and means an enzyme that catalyzes the conversion of L-xylulose to xylitol. L-xylulose reductase activity can be determined using methods known in the art (e.g., as described in U.S. Pat. No. 7,527,951).

Reference to “about” a value or parameter herein includes embodiments that are directed to that value or parameter per se. For example, description referring to “about X” includes the embodiment “X”. When used in combination with measured values, “about” includes a range that encompasses at least the uncertainty associated with the method of measuring the particular value, and can include a range of plus or minus two standard deviations around the stated value.

Likewise, reference to a gene or polypeptide that is “derived from” another gene or polypeptide X, includes the gene or polypeptide X.

As used herein and in the appended claims, the singular forms “a,” “or,” and “the” include plural referents unless the context clearly dictates otherwise.

It is understood that the embodiments described herein include “consisting” and/or “consisting essentially of” embodiments. As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments.

### DETAILED DESCRIPTION

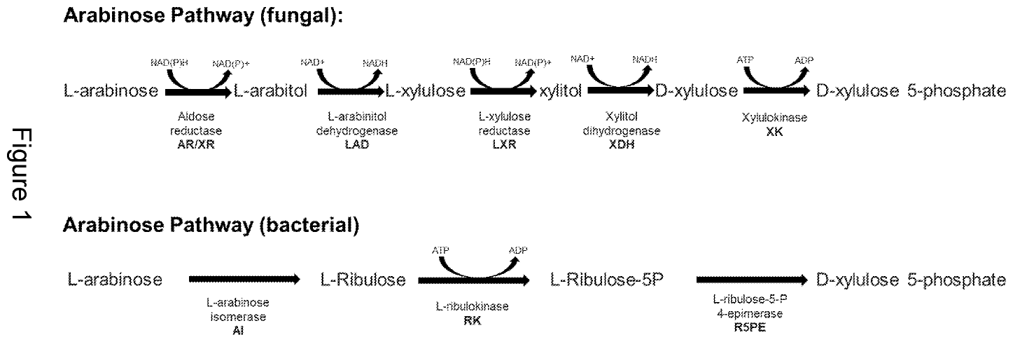

Described herein, inter alia, are host cells/fermentation organisms, and methods for producing a fermentation product, such as ethanol, from starch or cellulosic containing material. The Applicant surprisingly found a cluster of related L-xylulose reductase (LXR) genes that, when expressed in a yeast host, can provide exceptional arabinose utilization. These related genes are structurally distinct from those LXRs used in the art, as described in more detail below, which the Applicant believed is responsible for their unexpected superior performance. The Applicant also found that yeast can utilize arabinose in a fungal arabinose pathway that expresses the described heterologous LXR gene together with a heterologous aldose reductase (AR) and L-arabinitol 4-dehydrogenase (LAD), but without the need for expressing other heterologous genes of the pathway (See FIG. 1).

In one aspect is a method of producing a fermentation product from a starch-containing or cellulosic-containing material comprising:

(a) saccharifying the starch-containing or cellulosic-containing material; and
(b) fermenting the saccharified material of step (a) with a recombinant host cell;

wherein the cell has an active arabinose fermentation pathway and comprises:

(i) a heterologous polynucleotide encoding an aldose reductase (AR),

(ii) a heterologous polynucleotide encoding a L-arabinitol 4-dehydrogenase (LAD), and

(iii) a heterologous polynucleotide encoding a L-xylulose reductase (LXR);

wherein the L-xylulose reductase (LXR) has a mature polypeptide sequence with at least 60%, e.g., at least 65%, 70%, 75%, 80%, 85%, 90%, 95%, 97%, 98%, 99%, or 100% sequence identity to an LXR described here (e.g., any one of SEQ ID NOs: 297, 300, 302 and 304).

Steps a) and b) may be carried out either sequentially or simultaneously (SSF). In one embodiment, steps a) and b) are carried out simultaneously (SSF). In another embodiment, steps a) and b) are carried out sequentially.

In some embodiments of the methods described herein, fermentation of step (b) consumes a greater amount of arabinose e.g., at least 5%, 10%, 15%, 20%, 25%, 30%, 35%, 40%, 45%, 50%, 60%, 75% or 90% more when compared to the method using the same cell without the heterologous polynucleotide encoding a L-xylulose reductase (LXR) (e.g., under conditions described in the examples herein). In some embodiments, fermentation of step (b) consumes a greater amount of arabinose compared to the method using the same cell encoding the L-xylulose reductase (LXR) of SEQ ID NO: 305 (e.g., under conditions described in the examples herein). In some embodiments, more than 65%, e.g., at least 70%, 75%, 80%, 85%, 90%, 95% of arabinose in the medium is consumed.

### Host Cells and Fermenting Organisms

The host cells and fermenting organisms described herein may be derived from any host cell known to the skilled artisan, such as a cell capable of producing a fermentation product (e.g., ethanol). As used herein, a “derivative” of strain is derived from a referenced strain, such as through mutagenesis, recombinant DNA technology, mating, cell fusion, or cytoduction between yeast strains. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species.

The host cells described herein can be from any suitable host, such as a yeast strain, including, but not limited to, a Saccharomyces, Rhodotorula, Schizosaccharomyces, Kluyveromyces, Pichia, Hansenula, Rhodospordium, Candida, Yarrowia, Lipomyces, Cryptococcus, or Dekkera sp. cell. In particular, Saccharomyces host cells are contemplated, such as Saccharomyces cerevisiae, bayanus or cadsbergensis cells. Preferably, the yeast cell is a Saccharomyces cerevisiae cell. Suitable cells can, for example, be derived from commercially available strains and polyploid or aneuploid industrial strains, including but not limited to those from Superstart™, THERMOSACC®, C5 FUEL™, XyloFerm®, etc. (Lallemand); RED STAR and ETHANOL RED® (Fermentis/Lesaffre); FALI (AB Mauri); Bakers Best Yeast, Bakers Compressed Yeast, etc. (Fleishmann's Yeast); BIOFERM AFT, XP, CF, and XR (North American Bioproducts Corp.); Turbo Yeast (Gert Strand AB); and FERMIOL® (DSM Specialties). Other useful yeast strains are available from biological depositories such as the American Type Culture Collection (ATCC) or the Deutsche Sammlung von Mikroorganismen und Zellkulturen GmbH (DSMZ), such as, e.g., BY4741 (e.g., ATCC 201388); Y108-1 (ATCC PTA.10567) and NRRL YB-1952 (ARS Culture Collection). Still other S. cerevisiae strains suitable as host cells DBY746, [Alpha][Eta]22, S150-2B, GPY55-15Ba, CEN.PK, USM21, TMB3500, TMB3400, VTT-A-63015, VTT-A-85068, VTT-c-79093 and their derivatives as well as Saccharomyces sp. 1400, 424A (LNH-ST), 259A (LNH-ST) and derivatives thereof. In one embodiment, the recombinant cell is a derivative of a strain Saccharomyces cerevisiae CIBTS1260 (deposited under Accession No. NRRL Y-50973 at the Agricultural Research Service Culture Collection (NRRL), Illinois 61604 U.S.A.).

The host cell or fermenting organism may be Saccharomyces strain, e.g., Saccharomyces cerevisiae strain produced using the method described and concerned in U.S. Pat. No. 8,257,959.

The strain may also be a derivative of Saccharomyces cerevisiae strain NMI V14/004037 (See, WO2015/143324 and WO02015/143317 each incorporated herein by reference), strain nos. V15/004035, V15/004036, and V15/004037 (See, WO02016/153924 incorporated herein by reference), strain nos. V15/001459, V15/001460, V15/001461 (See, WO02016/138437 incorporated herein by reference), strain no. NRRL Y67342 (See, WO02018/098381 incorporated herein by reference), strain nos. NRRL Y67549 and NRRL Y67700 (See, WO02019/161227 incorporated herein by reference), or any strain described in WO02017/087330 (incorporated herein by reference).

The fermenting organisms according to the invention have been generated in order to, e.g., improve fermentation yield and to improve process economy by cutting enzyme costs since part or all of the necessary enzymes needed to improve method performance are be produced by the fermenting organism.

The host cells and fermenting organisms described herein may utilize expression vectors comprising the coding sequence of one or more (e.g., two, several) heterologous genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

A construct or vector (or multiple constructs or vectors) comprising the one or more (e.g., two, several) heterologous genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one embodiment, the nucleic acid construct encoding the polypeptide of interest is operably linked to a promoter foreign to the polynucleotide. The promoters may be identical to or share a high degree of sequence identity (e.g., at least about 80%, at least about 85%, at least about 90%, at least about 95%, at least about 96%, at least about 97%, at least about 98%, or at least about 99%) with a selected native promoter.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, aldose reductase (AR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5-phosphate decarboxylase (URA3) genes. Other suitable promoters may be obtained from S. cerevisiae TDH3, HXT7, PGK1, RPL18B and CCW12 genes. Additional useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, at least about 96%, at least about 97%, at least about 98%, or at least about 99%) with the selected native terminator.

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL10 terminator). Other suitable terminators may be obtained from S. cerevisiae ENO2 or TEF1 genes. Additional useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a suitable leader sequence, when transcribed is a non-translated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used. Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra. Signal peptides are also described in U.S. Provisional application No. 62/883,519, filed Aug. 6, 2019 (incorporated herein by reference).

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like. Suitable markers for yeast host cells include, but are not limited to, ADE2, HIS3, LEU2, LYS2, MET3, TRP1, and URA3.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo. Examples of origins of replication for use in a yeast host cell are the 2 micron origin of replication, ARS1, ARS4, the combination of ARS1 and CEN3, and the combination of ARS4 and CEN6.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, Molecular Cloning, A Laboratory Manual, 2d edition, Cold Spring Harbor, New York).

Additional procedures and techniques known in the art for the preparation of recombinant cells for ethanol fermentation, are described in, e.g., WO2016/045569, the content of which is hereby incorporated by reference.

The host cell or fermenting organism may be in the form of a composition comprising a host cell or fermenting organism (e.g., a yeast strain described herein) and a naturally occurring and/or a non-naturally occurring component.

The host cell or fermenting organism described herein may be in any viable form, including crumbled, dry, including active dry and instant, compressed, cream (liquid) form etc. In one embodiment, the host cell or fermenting organism (e.g., a Saccharomyces cerevisiae yeast strain) is dry yeast, such as active dry yeast or instant yeast. In one embodiment, the host cell or fermenting organism (e.g., a Saccharomyces cerevisiae yeast strain) is crumbled yeast. In one embodiment, the host cell or fermenting organism (e.g., a Saccharomyces cerevisiae yeast strain) is compressed yeast. In one embodiment, the host cell or fermenting organism (e.g., a Saccharomyces cerevisiae yeast strain) is cream yeast.

In one embodiment is a composition comprising a host cell or fermenting organism described herein (e.g., a Saccharomyces cerevisiae yeast strain), and one or more of the component selected from the group consisting of: surfactants, emulsifiers, gums, swelling agent, and antioxidants and other processing aids.

The compositions described herein may comprise a host cell or fermenting organism described herein (e.g., a Saccharomyces cerevisiae yeast strain) and any suitable surfactants.

In one embodiment, the surfactant(s) is/are an anionic surfactant, cationic surfactant, and/or nonionic surfactant.

The compositions described herein may comprise a host cell or fermenting organism described herein (e.g., a Saccharomyces cerevisiae yeast strain) and any suitable emulsifier.

In one embodiment, the emulsifier is a fatty-acid ester of sorbitan. In one embodiment, the emulsifier is selected from the group of sorbitan monostearate (SMS), citric acid esters of monodiglycerides, polyglycerolester, fatty acid esters of propylene glycol.

In one embodiment, the composition comprises a host cell or fermenting organism described herein (e.g., a Saccharomyces cerevisiae yeast strain), and Olindronal SMS, Olindronal SK, or Olindronal SPL including composition concerned in European Patent No. 1,724,336 (hereby incorporated by reference). These products are commercially available from Bussetti, Austria, for active dry yeast.

The compositions described herein may comprise a host cell or fermenting organism described herein (e.g., a Saccharomyces cerevisiae yeast strain) and any suitable gum. In one embodiment, the gum is selected from the group of carob, guar, tragacanth, arabic, xanthan and acacia gum, in particular for cream, compressed and dry yeast.

The compositions described herein may comprise a host cell or fermenting organism described herein (e.g., a Saccharomyces cerevisiae yeast strain) and any suitable swelling agent. In one embodiment, the swelling agent is methyl cellulose or carboxymethyl cellulose.

The compositions described herein may comprise a host cell or fermenting organism described herein (e.g., a Saccharomyces cerevisiae yeast strain) and any suitable anti-oxidant. In one embodiment, the antioxidant is butylated hydroxyanisol (BHA) and/or butylated hydroxytoluene (BHT), or ascorbic acid (vitamin C), particular for active dry yeast.

### Active Arabinose Fermentation Pathway

The host cells or fermenting organisms described herein (e.g., yeast cells) comprise an active arabinose fermentation pathway expressing a heterologous L-xylulose reductase (LXR).

As shown in FIG. 1, L-xylulose reductase (LXR) is an enzyme used in a “fungal pathway” that proceeds from L-arabinose to D-xyluluose-5-phosphate, where the L-xylulose reductase provides enzymatic activity for converting L-xylulose to xylitol. This pathway also includes transformations from enzymes aldose reductase (AR), L-arabinitol 4-dehydrogenase (LAD), xylitol dehydrogenase (XDH, also known as D-xylulose reductase) and xylulokinase (XK). As described in the examples, the Applicant has shown that arabinose can be consumed through heterologous expression of AR, LAD and LXR, even without heterologous expression of remaining enzymes of the pathway XDH and XK.

However, strains expressing the LXR previously used and described in U.S. Pat. No. 7,527,951 (Trichoderma reesei, SEQ ID NO: 305) were unable to grow on arabinose agar plates even with heterologous expression of the full pathway (See Example 1). Upon screening yeasts expressing putative LXRs (Table 1) on agarose, the Applicant found that expression of most putative LXRs showed no growth (SEQ ID NOs: 298, 299, 303, 305, 307, 308) or only minimal growth (SEQ ID NOs: 301 and 306), but a subset of LXRs showed substantial growth (SEQ ID NOs: 297, 300, 302, 304).

TABLE 1

| | | SEQ ID NO: |
|---|---|---|
| Donor Organism | Gene ID | (mature polypeptide) |
| A. brasiliensis | EFP7G1BZ1 | 297 |
| C. sorboxylosa | EFP5PTNSX | 298 |
| B. anomalus | EFP480208 | 299 |
| T. leycettanus | EFP3RCJ9K | 300 |
| K. capsulata | EFP2CH8R5 | 301 |
| A. aculeatus | EFP2BNSMN | 302 |
| P. membranifaciens | EFP16N8ZZ | 303 |
| A. niger | G3YG17 | 304 |
| T. reesei | Q8NK50 | 305 |
| A. monospora | Q70FD1 | 306 |
| C. arabinofermentans | A0A1E4T269 | 307 |
| O. parapolymorpha | E7R859 | 308 |

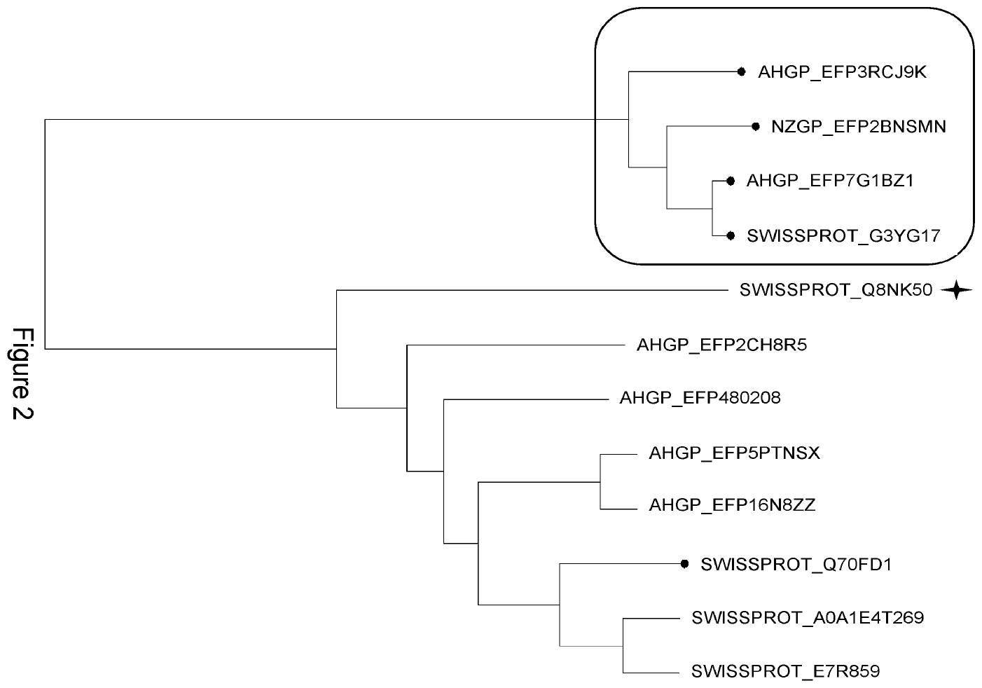

Those LXRs that resulted in substantial growth were found to be genetically distinct from the remaining LXRs that showed only minimal or no cell growth under the same conditions, including the T. reesei LXR of SEQ ID NO: 305 previously described in U.S. Pat. No. 7,527,951. Table 2 below shows a matrix of sequence homology among the screened LXRs. More than 70% sequence identity is shared among the best LXRs of SEQ ID NOs: 297, 300, 302, 304, while each share less than 20% sequence identity to the remaining poor performing LXRs (including only about 16% identity to previously described T. reesei LXR of SEQ ID NO: 305). FIG. 2 shows a functional diversity tree for the expressed LXRs. The cluster of LXRs that provided substantial cell growth on arabinose agar plates is indicated with a box.

TABLE 2

| SEQ | SEQ ID NO: | | | | | | | | | | | |
|---|---|---|---|---|---|---|---|---|---|---|---|---|
| ID NO: | 302 | 304 | 297 | 300 | 306 | 301 | 308 | 305 | 299 | 303 | 307 | 298 |
| 302 | 100 | 78 | 79 | 70 | 19 | 18 | 18 | 16 | 17 | 18 | 19 | 17 |
| 304 | 78 | 100 | 91 | 71 | 17 | 18 | 18 | 16 | 16 | 17 | 18 | 16 |
| 297 | 79 | 91 | 100 | 71 | 18 | 18 | 18 | 16 | 16 | 17 | 18 | 16 |
| 300 | 70 | 71 | 71 | 100 | 19 | 17 | 19 | 16 | 18 | 19 | 19 | 18 |
| 306 | 19 | 17 | 18 | 19 | 100 | 59 | 70 | 33 | 59 | 59 | 68 | 60 |
| 301 | 18 | 18 | 18 | 17 | 59 | 100 | 64 | 32 | 56 | 55 | 61 | 53 |
| 308 | 18 | 18 | 18 | 19 | 70 | 64 | 100 | 34 | 65 | 68 | 79 | 68 |
| 305 | 16 | 16 | 16 | 16 | 33 | 32 | 34 | 100 | 32 | 31 | 33 | 31 |
| 299 | 17 | 16 | 16 | 18 | 59 | 56 | 65 | 32 | 100 | 58 | 64 | 60 |
| 303 | 18 | 17 | 17 | 19 | 59 | 55 | 68 | 31 | 58 | 100 | 67 | 82 |
| 307 | 19 | 18 | 18 | 19 | 68 | 61 | 79 | 33 | 64 | 67 | 100 | 66 |
| 298 | 17 | 16 | 16 | 18 | 60 | 53 | 68 | 31 | 60 | 82 | 66 | 100 |

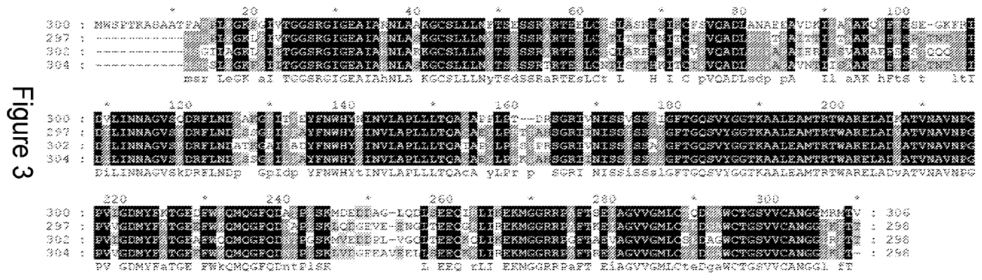

A Multiple Sequence Alignment of SEQ ID NOs: 297, 300, 302, and 304 is shown in FIG. 3 to illustrate the common residues among this identified cluster of LXRs.

In one embodiment, the L-xylulose reductase (LXR) comprises or consists of the amino acid sequence of any one of SEQ ID NOs: 297, 300, 302 and 304. In another embodiment, the LXR is a fragment of the LXR of any one of SEQ ID NOs: 297, 300, 302 and 304, wherein, e.g., the fragment has LXR activity. In one embodiment, the number of amino acid residues in the fragment is at least 75%, e.g., at least 80%, 85%, 90%, or 95% of the number of amino acid residues in referenced full length LXR (e.g. any one of SEQ ID NOs: 297, 300, 302 and 304). In other embodiments, the LXR may comprise the catalytic domain of any LXR described or referenced herein (e.g., the catalytic domain of any one of SEQ ID NOs: 297, 300, 302 and 304).

In some embodiments, the host cells or fermenting organisms comprising a heterologous polynucleotide encoding a L-xylulose reductase (LXR) have an increased level of LXR activity compared to the host cells without the heterologous polynucleotide encoding the LXR, when cultivated under the same conditions. In some embodiments, the host cells have an increased level of LXR activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the LXR, when cultivated under the same conditions.

The L-xylulose reductase (LXR) may be a variant or homolog (e.g. natural or synthetic) of any one of the LXRs described supra (e.g., any one of SEQ ID NOs: 297, 300, 302 and 304). In one embodiment, the LXR has at least 60%, e.g., at least 65%, 70%, 75%, 80%, 85%, 90%, 95%, 97%, 98%, 99%, or 100% sequence identity to any one of the LXRs described supra (e.g., any one of SEQ ID NOs: 297, 300, 302 and 304).

The L-xylulose reductase (LXR) may be a bacterial LXR. For example, the LXR may be derived from a Gram-positive bacterium such as a Bacillus, Clostridium, Enterococcus, Geobacillus, Lactobacillus, Lactococcus, Oceanobacillus, Staphylococcus, Streptococcus, or Streptomyces, or a Gram-negative bacterium such as a Campylobacter, E. coli, Flavobacterium, Fusobacterium, Helicobacter, Ilyobacter, Neisseria, Pseudomonas, Salmonella, or Ureaplasma.

In one embodiment, the L-xylulose reductase (LXR) is derived from Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.

In another embodiment, the L-xylulose reductase (LXR) is derived from Streptococcus 20 equisimilis, Streptococcus pyogenes, Streptococcus uberis, or Streptococcus equi subsp. Zooepidemicus.

In another embodiment, the L-xylulose reductase (LXR) is derived from Streptomyces achromogenes, Streptomyces avermitilis, Streptomyces coelicolor, Streptomyces griseus, or Streptomyces lividans.

The L-xylulose reductase (LXR) may be a fungal LXR. For example, the LXR may be derived from a yeast such as a Candida, Kluyveomyces, Pichia, Saccharomyces, Schizosaccharomyces, Yarrowia or Issatchenkia; or derived from a filamentous fungus such as an Acremonium, Agaricus, Altemaria, Aspergillus, Aureobasidium, Botryosphaeria, Ceriponopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phaneochaete, Piromyces, Poitasia, Pseudoplectania, Pseudotrichonympha, Rhizomucor, Schizophyllum, Scytalidium, Talaomyces, Thermoascus, Thielavia, Tolypocladium, Trichoderma, Trichophaea, Verticillium, Volvariella, or Xylaria.

In another embodiment, the L-xylulose reductase (LXR) is derived from Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis.

In another embodiment, the L-xylulose reductase (LXR) is derived from Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusanum roseum, Fusanum sambucinum, Fusanum sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The L-xylulose reductase (LXR) coding sequences described or referenced herein, or a subsequence thereof, as well as the LXR described or referenced herein, or a fragment thereof, may be used to design nucleic acid probes to identify and clone DNA encoding a LXR from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a sugar transporter. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with a coding sequence, or a subsequence thereof, the carrier material is used in a Southern blot.

In one embodiment, the nucleic acid probe is a polynucleotide, or subsequence thereof, that encodes the L-xylulose reductase (LXR) of any one of SEQ ID NOs: 297, 300, 302 and 304, or a fragment thereof.

For purposes of the probes described above, hybridization indicates that the polynucleotide hybridizes to a labeled nucleic acid probe, or the full-length complementary strand thereof, or a subsequence of the foregoing; under very low to very high stringency conditions. Molecules to which the nucleic acid probe hybridizes under these conditions can be detected using, for example, X-ray film. Stringency and washing conditions are defined as described supra.

In one embodiment, the L-xylulose reductase (LXR) is encoded by a polynucleotide that hybridizes under at least low stringency conditions, e.g., medium stringency conditions, medium-high stringency conditions, high stringency conditions, or very high stringency conditions with the full-length complementary strand of the coding sequence for any one of the LXRs described or referenced herein (e.g., SEQ ID NOs: 297, 300, 302 and 304). (Sambrook et al., 1989, Molecular Cloning, A Laboratory Manual, 2d edition, Cold Spring Harbor, New York).

The L-xylulose reductase (LXR) may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding an LXR may then be derived by similarly screening a genomic or cDNA library of another microorganism or mixed DNA sample.

Once a polynucleotide encoding a L-xylulose reductase (LXR) has been detected with a suitable probe as described herein, the sequence may be isolated or cloned by utilizing techniques that are known to those of ordinary skill in the art (See, e.g., Sambrook et al., 1989, supra). Techniques used to isolate or clone polynucleotides encoding LXRs include isolation from genomic DNA, preparation from cDNA, or a combination thereof. The cloning of the polynucleotides from such genomic DNA can be affected, e.g., by using the well-known polymerase chain reaction (PCR) or antibody screening of expression libraries to detect cloned DNA fragments with shares structural features (See, e.g., Innis et al., 1990, PCR: A Guide to Methods and Application, Academic Press, New York). Other nucleic acid amplification procedures such as ligase chain reaction (LCR), ligated activated transcription (LAT) and nucleotide sequence-based amplification (NASBA) may be used.

In one embodiment, the L-xylulose reductase (LXR) sequence differs by no more than ten amino acids, e.g., by no more than five amino acids, by no more than four amino acids, by no more than three amino acids, by no more than two amino acids, or by one amino acid from the amino acid sequence of any one of the LXRs described supra (e.g., any one of SEQ ID NOs: 297, 300, 302 and 304). In one embodiment, the LXR has an amino acid substitution, deletion, and/or insertion of one or more (e.g., two, several) of amino acid sequence of any one of the LXRs described supra (e.g., any one of SEQ ID NOs: 297, 300, 302 and 304). In some embodiments, the total number of amino acid substitutions, deletions and/or insertions is not more than 10, e.g., not more than 9, 8, 7, 6, 5, 4, 3, 2, or 1.

The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H.

Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the LXRs, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site or other biological interaction can also be determined by physical analysis of structure, as determined by such techniques as nuclear magnetic resonance, crystallography, electron diffraction, or photoaffinity labeling, in conjunction with mutation of putative contact site amino acids (See, for example, de Vos et al., 1992, Science 255: 306-312; Smith et al., 1992, J. Mol. Biol. 224: 899-904; Wlodaver et al., 1992, FEBS Lett. 309: 59-64). The identities of essential amino acids can also be inferred from analysis of identities with other LXRs that are related to the referenced LXR.

Additional guidance on the structure-activity relationship of the L-xylulose reductases (LXRs) herein can be determined using multiple sequence alignment (MSA) techniques well-known in the art. Based on the teachings herein, the skilled artisan could make similar alignments with any number of LXRs described herein or known in the art (e.g., an MSA of SEQ ID NOs: 297, 300, 302 and 304, as shown in FIG. 3). Such alignments aid the skilled artisan to determine potentially relevant domains (e.g., binding domains or catalytic domains), as well as which amino acid residues are conserved and not conserved among the different transporter sequences. It is appreciated in the art that changing an amino acid that is conserved at a particular position between disclosed polypeptides will more likely result in a change in biological activity (Bowie et al., 1990, Science 247: 1306-1310: “Residues that are directly involved in protein functions such as binding or catalysis will certainly be among the most conserved). In contrast, substituting an amino acid that is not highly conserved among the polypeptides will not likely or significantly alter the biological activity.

Even further guidance on the structure-activity relationship for the skilled artisan can be found in published x-ray crystallography studies known in the art.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO95/17413; or WO095/22625. Other methods that can be used include error-prone PCR, phage display (e.g., Lowman et al., 1991, Biochemistry 30: 10832-10837; U.S. Pat. No. 5,223,409; WO92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active GAPNs can be recovered from the host cells and rapidly sequenced using standard methods in the art. These methods allow the rapid determination of the importance of individual amino acid residues in a polypeptide.

In another embodiment, the heterologous polynucleotide encoding the L-xylulose reductase (LXR) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to the coding sequence of any one of the LXRs described supra (e.g., any one of SEQ ID NOs: 297, 300, 302 and 304).

In one embodiment, the heterokogous polynucleotide encoding the L-xylulose reductase (LXR) comprises or consists of the coding sequence of any one of the LXRs described supra (e.g., any one of SEQ ID NOs: 297, 300, 302 and 304). In another embodiment, the heterologous polynucleotide encoding the LXR comprises a subsequence of the coding sequence of any one of the LXRs described supra (e.g., any one of SEQ ID NOs: 297, 300, 302 and 304) wherein the subsequence encodes a polypeptide having LXR activity. In another embodiment, the number of nucleotides residues in the coding subsequence is at least 75%, e.g., at least 80%, 85%, 90%, or 95% of the number of the referenced coding sequence.

The referenced coding sequence of any related aspect or embodiment described herein can be the native coding sequence or a degenerate sequence, such as a codon-optimized coding sequence designed for use in a particular host cell (e.g., optimized for expression in Saccharomyces cerevisiae). Codon-optimization for expression in yeast cells is known in the art (e.g., U.S. Pat. No. 8,326,547).

The L-xylulose reductase (LXR) may be a fused polypeptide or cleavable fusion polypeptide in which another polypeptide is fused at the N-terminus or the C-terminus of the LXR. A fused polypeptide may be produced by fusing a polynucleotide encoding another polypeptide to a polynucleotide encoding the LXR. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

In addition to the heterologous L-xylulose reductase (LXR), any suitable arabinose fermentation pathway gene, endogenous or heterologous, may be used and expressed in sufficient amount to produce an enzyme involved in the pathway. With the complete genome sequence available for now numerous microorganism genomes and a variety of yeast, fungi, plant, and mammalian genomes, the identification of genes encoding the selected arabinose fermentation pathway enzymatic activities taught herein is routine and well known in the art for a selected host. For example, suitable homologues, orthologs, paralogs and nonorthologous gene displacements of known genes, and the interchange of genetic alterations between organisms can be identified in related or distant host to a selected host.

For host cells without a known genome sequence, sequences for genes of interest (either as overexpression candidates or as insertion sites) can typically be obtained using techniques known in the art. Routine experimental design can be employed to test expression of various genes and activity of various enzymes, including genes and enzymes that function in a pentose fermentation pathway. Experiments may be conducted wherein each enzyme is expressed in the cell individually and in blocks of enzymes up to and including preferably all pathway enzymes, to establish which are needed (or desired) for improved pentose fermentation. One illustrative experimental design tests expression of each individual enzyme as well as of each unique pair of enzymes, and further can test expression of all required enzymes, or each unique combination of enzymes. A number of approaches can be taken, as will be appreciated.

The host cells of the invention can be produced by introducing heterologous polynucleotides encoding one or more of the enzymes participating in an active arabinose fermentation pathway, as described below. As one in the art will appreciate, in some instances (e.g., depending on the selection of host) the heterologous expression of every gene shown in the active arabinose fermentation may not be required since a host cell may have endogenous enzymatic activity from one or more pathway genes. For example, if a chosen host is deficient in one or more enzymes of an active arabinose fermentation pathway, then heterologous polynucleotides for the deficient enzyme(s) are introduced into the host for subsequent expression. Alternatively, if the chosen host exhibits endogenous expression of some pathway genes, but is deficient in others, then an encoding polynucleotide is needed for the deficient enzyme(s) to achieve arabinose fermentation. Thus, a recombinant host cell of the invention can be produced by introducing heterologous polynucleotides to obtain the enzyme activities of a desired biosynthetic pathway or a desired biosynthetic pathway can be obtained by introducing one or more heterologous polynucleotides that, together with one or more endogenous enzymes, produces a desired product such as ethanol.

The host cells of the invention will include at least one heterologous polynucleotide encoding an L-xylulose reductase (LXR) described above and optionally up to all encoding heterologous polynucleotides for the arabinose fermentation pathway. For example, arabinose fermentation can be established in a host deficient in an arabinose fermentation pathway enzyme through heterologous expression of the corresponding polynucleotide. In a host deficient in all enzymes of an arabinose fermentation pathway, heterologous expression of all enzymes in the pathway can be included, although it is understood that all enzymes of a pathway can be expressed even if the host contains at least one of the pathway enzymes.

The enzymes of the selected active arabinose fermentation pathway, and activities thereof, can be detected using methods known in the art or as described herein. These detection methods may include use of specific antibodies, formation of an enzyme product, or disappearance of an enzyme substrate. See, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999); and Hanai et al., Appl. Environ. Microbiol. 73:7814-7818 (2007)).

In one embodiment, the host cell or fermenting organism (e.g., yeast cell) further comprises a heterologous polynucleotide encoding an aldose reductase (AR). An aldose reductase, as used herein, provides enzymatic activity for converting L-arabinose to L-arabitol, and may also have enzymatic activity for converting D-xylose to xylitol (known as a xylose reductase, XR). The aldose reductase may be any aldose reductase that is suitable for the host cells and the methods described herein, such as a naturally occurring aldose reductase or a variant thereof that retains aldose reductase activity. In one embodiment, the aldose reductase is present in the cytosol of the host cells.

In some embodiments, the host cells or fermenting organisms comprising a heterologous polynucleotide encoding an aldose reductase (AR) have an increased level of aldose reductase activity compared to the host cells without the heterokogous polynucleotide encoding the aldose reductase, when cultivated under the same conditions. In some embodiments, the host cells have an increased level of aldose reductase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the aldose reductase, when cultivated under the same conditions.

Exemplary aldose reductases (ARs) that can be used with the host cells and fermenting organisms, and methods of use described herein include, but are not limited to, the Aspergillus niger aldose reductase of SEQ ID NO: 281, the Aspergillus oryzae aldose reductase of SEQ ID NO: 282, the Magnaporthe oryzae aldose reductase of SEQ ID NO: 283, the Meyerozyma guilliermondii aldose reductase of SEQ ID NO: 284 and the Scheffersomyces stipitis aldose reductase of SEQ ID NO: 285. Additional polynucleotides encoding suitable aldose reductase may be obtained from microorganisms of any genus, including those readily available within the UniProtKB database. In one embodiment, the aldose reductase is a bacterial, a yeast, or a filamentous fungal aldose reductase, e.g., obtained from any of the microorganisms described or referenced herein, as described supra.

The aldose reductase (AR) coding sequences can also be used to design nucleic acid probes to identify and clone DNA encoding aldose reductases from strains of different genera or species, as described supra.

The polynucleotides encoding the aldose reductases (ARs) may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding aldose reductases (ARs) are described supra.

In one embodiment, the aldose reductase (AR) has a mature polypeptide sequence of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any aldose reductase described or referenced herein (e.g., the aldose reductase of SEQ ID NO: 281, 282, 283, 284 or 285). In one embodiment, the aldose reductase has a mature polypeptide sequence that differs by no more than ten amino acids, e.g., by no more than five amino acids, by no more than four amino acids, by no more than three amino acids, by no more than two amino acids, or by one amino acid from any aldose reductase described or referenced herein (e.g., the aldose reductase of SEQ ID NO: 281, 282, 283, 284 or 285). In one embodiment, the aldose reductase has a mature polypeptide sequence that comprises or consists of the amino acid sequence of any aldose reductase described or referenced herein (e.g., the aldose reductase of SEQ ID NO: 281, 282, 283, 284 or 285), allelic variant, or a fragment thereof having aldose reductase activity. In one embodiment, the aldose reductase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the aldose reductase (AR) has at least 20%, e.g., at least 40%, at least 50%, at least 60%, at least 70%, at least 80%, at least 90%, at least 95%, at least 96%, at least 97%, at least 98%, at least 99%, or 100% of the aldose reductase activity of any aldose reductase described or referenced herein (e.g., the aldose reductase of SEQ ID NO: 281, 282, 283, 284 or 285) under the same conditions.

In one embodiment, the aldose reductase (AR) coding sequence hybridizes under at least low stringency conditions, e.g., medium stringency conditions, medium-high stringency conditions, high stringency conditions, or very high stringency conditions with the full-length complementary strand of the coding sequence from any aldose reductase described or referenced herein (e.g., the aldose reductase of SEQ ID NO: 281, 282, 283, 284 or 285). In one embodiment, the aldose reductase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any aldose reductase described or referenced herein (e.g., the aldose reductase of SEQ ID NO: 281, 282, 283, 284, or 285).

In one embodiment, the heterologous polynucleotide encoding the aldose reductase (AR) comprises the coding sequence of any aldose reductase described or referenced herein (e.g., the aldose reductase of SEQ ID NO: 281, 282, 283, 284, or 285). In one embodiment, the heterologous polynucleotide encoding the aldose reductase comprises a subsequence of the coding sequence from any aldose reductase described or referenced herein, wherein the subsequence encodes a polypeptide having aldose reductase activity. In one embodiment, the number of nucleotides residues in the subsequence is at least 75%, e.g., at least 80%, 85%, 90%, or 95% of the number of the referenced coding sequence.

The aldose reductases (ARs) can also include fused polypeptides or cleavable fusion polypeptides, as described supra.

In one embodiment, the host cell or fermenting organism (e.g., yeast cell) further comprises a heterologous polynucleotide encoding an L-arabinitol 4-dehydrogenase (LAD). A L-arabinitol 4-dehydrogenase, as used herein, provides enzymatic activity for converting L-arabitol to L-xylulose. The L-arabinitol 4-dehydrogenase may be any L-arabinitol 4-dehydrogenase that is suitable for the host cells and the methods described herein, such as a naturally occurring L-arabinitol 4-dehydrogenase or a variant thereof that retains L-arabinitol 4-dehydrogenase activity. In one embodiment, the L-arabinitol 4-dehydrogenase is present in the cytosol of the host cells.

In some embodiments, the host cells or fermenting organisms comprising a heterologous polynucleotide encoding a L-arabinitol 4-dehydrogenase (LAD) have an increased level of L-arabinitol 4-dehydrogenase activity compared to the host cells without the heterologous polynucleotide encoding the L-arabinitol 4-dehydrogenase, when cultivated under the same conditions. In some embodiments, the host cells have an increased level of L-arabinitol 4-dehydrogenase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the L-arabinitol 4-dehydrogenase, when cultivated under the same conditions.

Exemplary L-arabinitol 4-dehydrogenases (LADs) that can be used with the host cells and fermenting organisms, and methods of use described herein include, but are not limited to, the Meyerozyma caribbica LAD of SEQ ID NO: 286, the Trichoderma reesei LAD of SEQ ID NO: 287, the Meyerozyma guilliermondii LAD of SEQ ID NO: 288, the Candida arabinofermentans LAD of SEQ ID NO: 289, the Candida carpophila LAD of SEQ ID NO: 290, the Talaromyces emersonii LAD of SEQ ID NO: 291, the Aspergillus oryzae LAD of SEQ ID NO: 292, the Neurospora crassa LAD of SEQ ID NO: 293, the Trichoderma reesei LAD of SEQ ID NO: 294, the Aspergillus niger LAD of SEQ ID NO: 295 and the Penicillium rubens LAD of SEQ ID NO: 296. Additional polynucleotides encoding suitable L-arabinitol 4-dehydrogenases may be obtained from microorganisms of any genus, including those readily available within the UniProtKB database. In one embodiment, the L-arabinitol 4-dehydrogenase is a bacterial, a yeast, or a filamentous fungal L-arabinitol 4-dehydrogenase, e.g., obtained from any of the microorganisms described or referenced herein, as described supra.

The L-arabinitol 4-dehydrogenase (LAD) coding sequences can also be used to design nucleic acid probes to identify and clone DNA encoding L-arabinitol 4-dehydrogenases from strains of different genera or species, as described supra.

The polynucleotides encoding L-arabinitol 4-dehydrogenases (LADs) may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding L-arabinitol 4-dehydrogenases (LADs) are described supra.

In one embodiment, the L-arabinitol 4-dehydrogenase (LAD) has a mature polypeptide sequence of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296). In one embodiment, the L-arabinitol 4-dehydrogenase has a mature polypeptide sequence that differs by no more than ten amino acids, e.g., by no more than five amino acids, by no more than four amino acids, by no more than three amino acids, by no more than two amino acids, or by one amino acid from any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296). In one embodiment, the L-arabinitol 4-dehydrogenase has a mature polypeptide sequence that comprises or consists of the amino acid sequence of any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296), allelic variant, or a fragment thereof having L-arabinitol 4-dehydrogenase activity. In one embodiment, the L-arabinitol 4-dehydrogenase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the L-arabinitol 4-dehydrogenase (LAD) has at least 20%, e.g., at least 40%, at least 50%, at least 60%, at least 70%, at least 80%, at least 90%, at least 95%, at least 96%, at least 97%, at least 98%, at least 99%, or 100% of the L-arabinitol 4-dehydrogenase activity of any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296) under the same conditions.

In one embodiment, the L-arabinitol 4-dehydrogenase (LAD) coding sequence hybridizes under at least low stringency conditions, e.g., medium stringency conditions, medium-high stringency conditions, high stringency conditions, or very high stringency conditions with the full-length complementary strand of the coding sequence from any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296). In one embodiment, the L-arabinitol 4-dehydrogenase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296).

In one embodiment, the heterologous polynucleotide encoding the L-arabinitol 4-dehydrogenase (LAD) comprises the coding sequence of any L-arabinitol 4-dehydrogenase described or referenced herein (e.g., the L-arabinitol 4-dehydrogenase of SEQ ID NO: 286, 287, 288, 289, 290, 291, 292, 293, 294, 295 or 296). In one embodiment, the heterologous polynucleotide encoding the L-arabinitol 4-dehydrogenase comprises a subsequence of the coding sequence from any L-arabinitol 4-dehydrogenase described or referenced herein, wherein the subsequence encodes a polypeptide having L-arabinitol 4-dehydrogenase activity. In one embodiment, the number of nucleotides residues in the subsequence is at least 75%, e.g., at least 80%, 85%, 90%, or 95% of the number of the referenced coding sequence.

The L-arabinitol 4-dehydrogenases (LADs) can also include fused polypeptides or cleavable fusion polypeptides, as described supra.

In one embodiment, the host cell or fermenting organism (e.g., yeast cell) further comprises a heterologous polynucleotide encoding a xylitol dehydrogenase (XDH). A xylitol dehydrogenase, as used herein, provides enzymatic activity for converting xylitol to D-xylulose. The xylitol dehydrogenase may be any xylitol dehydrogenase that is suitable for the host cells and the methods described herein, such as a naturally occurring xylitol dehydrogenase or a variant thereof that retains xylitol dehydrogenase activity. In one embodiment, the xylitol dehydrogenase is present in the cytosol of the host cells.

In some embodiments, the host cells or fermenting organisms comprising a heterologous polynucleotide encoding a xylitol dehydrogenase (XDH) have an increased level of xylitol dehydrogenase activity compared to the host cells without the heterologous polynucleotide encoding the xylitol dehydrogenase, when cultivated under the same conditions. In some embodiments, the host cells have an increased level of xylitol dehydrogenase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylitol dehydrogenase, when cultivated under the same conditions.

Exemplary xylitol dehydrogenases (XDHs) that can be used with the host cells and fermenting organisms, and methods of use described herein include, but are not limited to, the Scheffersomyces stipitis xylitol dehydrogenase of SEQ ID NO: 309, the Trichoderma reesei xylitol dehydrogenase (Wang et al., 1998, Chin. J. Biotechnol. 14, 179-185), the Pichia stipitis xylitol dehydrogenase (Karhumaa et al, 2007, Microb Cell Fact. 6, 5), as well as other yeast xylitol dehydrogenases described in the art, such as XDHs from S. cerevisiae (Richard et. al., 1999, FEBS Letters 457, 135-138), C. didensiae, C. intermediae, C. parapsilosis, C. silvanoru, C. tropicalis, KI. Marxsianus, P. guilliermondii, T. molishiama, Pa. tannophilus, and C. shehatae (Yablochkova et al, 2003, Microbiology 72(4), 414-417). Additional polynucleotides encoding suitable xylitol dehydrogenases may be obtained from microorganisms of any genus, including those readily available within the UniProtKB database. In one embodiment, the xylitol dehydrogenase is a bacterial, a yeast, or a filamentous fungal xylitol dehydrogenase, e.g., obtained from any of the microorganisms described or referenced herein, as described supra.

The xylitol dehydrogenase (XDH) coding sequences can also be used to design nucleic acid probes to identify and clone DNA encoding xylitol dehydrogenases from strains of different genera or species, as described supra.

The polynucleotides encoding xylitol dehydrogenases (XDHs) may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding xylitol dehydrogenases (XDHs) are described supra.

In one embodiment, the xylitol dehydrogenase (XDH) has a mature polypeptide sequence of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylitol dehydrogenase described or referenced herein (e.g., the Scheffersomyces stipitis xylitol dehydrogenase of SEQ ID NO: 309). In one embodiment, the xylitol dehydrogenase has a mature polypeptide sequence that differs by no more than ten amino acids, e.g., by no more than five amino acids, by no more than four amino acids, by no more than three amino acids, by no more than two amino acids, or by one amino acid from any xylitol dehydrogenase described or referenced herein (e.g., the Scheffersomyces stipitis xylitol dehydrogenase of SEQ ID NO: 309). In one embodiment, the xylitol dehydrogenase has a mature polypeptide sequence that comprises or consists of the amino acid sequence of any xylitol dehydrogenase described or referenced herein (e.g., the Scheffersomyces stipitis xylitol dehydrogenase of SEQ ID NO: 309), allelic variant, or a fragment thereof having xylitol dehydrogenase activity.

In one embodiment, the xylitol dehydrogenase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylitol dehydrogenase (XDH) has at least 20%, e.g., at least 40%, at least 50%, at least 60%, at least 70%, at least 80%, at least 90%, at least 95%, at least 96%, at least 97%, at least 98%, at least 99%, or 100% of the xylitol dehydrogenase activity of any xylitol dehydrogenase described or referenced herein (e.g., the Scheffersomyces stipitis xylitol dehydrogenase of SEQ ID NO: 309) under the same conditions.

In one embodiment, the xylitol dehydrogenase (XDH) coding sequence hybridizes under at least low stringency conditions, e.g., medium stringency conditions, medium-high stringency conditions, high stringency conditions, or very high stringency conditions with the full-length complementary strand of the coding sequence from any xylitol dehydrogenase described or referenced herein (e.g., the Scheffersomyces stipitis xylitol dehydrogenase of SEQ ID NO: 309). In one embodiment, the xylitol dehydrogenase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylitol dehydrogenase described or referenced herein (e.g., the Scheffersomyces stipitis xylitol dehydrogenase of SEQ ID NO: 309).

In one embodiment, the heterologous polynucleotide encoding the xylitol dehydrogenase (XDH) comprises the coding sequence of any xylitol dehydrogenase described or referenced herein (e.g., the Scheffersomyces stipitis xylitol dehydrogenase of SEQ ID NO: 309). In one embodiment, the heterologous polynucleotide encoding the xylitol dehydrogenase comprises a subsequence of the coding sequence from any xylitol dehydrogenase described or referenced herein, wherein the subsequence encodes a polypeptide having xylitol dehydrogenase activity. In one embodiment, the number of nucleotides residues in the subsequence is at least 75%, e.g., at least 80%, 85%, 90%, or 95% of the number of the referenced coding sequence.

The xylitol dehydrogenases (XDHs) can also include fused polypeptides or cleavable fusion polypeptides, as described supra.

In one embodiment, the host cell or fermenting organism (e.g., yeast cell) further comprises a heterologous polynucleotide encoding a xylulokinase (XK). A xylulokinase, as used herein, provides enzymatic activity for converting D-xylulose to xylulose 5-phosphate.

The xylulokinase may be any xylulokinase that is suitable for the host cells and the methods described herein, such as a naturally occurring xylulokinase or a variant thereof that retains xylulokinase activity. In one embodiment, the xylulokinase is present in the cytosol of the host cells.

In some embodiments, the host cells or fermenting organisms comprising a heterologous polynucleotide encoding a xylulokinase (XK) have an increased level of xylulokinase activity compared to the host cells without the heterologous polynucleotide encoding the xylulokinase, when cultivated under the same conditions. In some embodiments, the host cells have an increased level of xylulokinase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylulokinase, when cultivated under the same conditions.

Exemplary xylulokinases (XKs) that can be used with the host cells and fermenting organisms, and methods of use described herein include, but are not limited to, the Saccharomyces cerevisiae xylulokinase of SEQ ID NO: 75, the Scheffersomyces stipitis xylulokinase of SEQ ID NO: 310 and the Aspergillus niger xylulokinase of SEQ ID NO: 311.

Additional xylulokinases are known in the art. Additional polynucleotides encoding suitable xylulokinases may be obtained from microorganisms of any genus, including those readily available within the UniProtKB database. In one embodiment, the xylulokinases is a bacterial, a yeast, or a filamentous fungal xylulokinase, e.g., obtained from any of the microorganisms described or referenced herein, as described supra.

The xylulokinase (XK) coding sequences can also be used to design nucleic acid probes to identify and clone DNA encoding xylulokinases from strains of different genera or species, as described supra.

The polynucleotides encoding xylulokinases (XK) may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding xylulokinases (XKs) are described supra.

In one embodiment, the xylulokinase (XK) has a mature polypeptide sequence of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylulokinase described or referenced herein (e.g., the xylulokinase of SEQ ID NO: 75, 310 or 311). In one embodiment, the xylulokinase has a mature polypeptide sequence that differs by no more than ten amino acids, e.g., by no more than five amino acids, by no more than four amino acids, by no more than three amino acids, by no more than two amino acids, or by one amino acid from any xylulokinase described or referenced herein (e.g., the xylulokinase of SEQ ID NO: 75, 310 or 311). In one embodiment, the xylulokinase has a mature polypeptide sequence that comprises or consists of the amino acid sequence of any xylulokinase described or referenced herein (e.g., the xylulokinase of SEQ ID NO: 75, 310 or 311), allelic variant, or a fragment thereof having xylulokinase activity. In one embodiment, the xylulokinase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylulokinase (XK) has at least 20%, e.g., at least 40%, at least 50%, at least 60%, at least 70%, at least 80%, at least 90%, at least 95%, at least 96%, at least 97%, at least 98%, at least 99%, or 100% of the xylulokinase activity of any xylulokinase described or referenced herein (e.g., the xylulokinase of SEQ ID NO: 75, 310 or 311) under the same conditions.

In one embodiment, the xylulokinase (XK) coding sequence hybridizes under at least low stringency conditions, e.g., medium stringency conditions, medium-high stringency conditions, high stringency conditions, or very high stringency conditions with the full-length complementary strand of the coding sequence from any xylulokinase described or referenced herein (e.g., the xylulokinase of SEQ ID NO: 75, 310 or 311). In one embodiment, the xylulokinase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylulokinase described or referenced herein (e.g., the xylulokinase of SEQ ID NO: 75, 310 or 311).

In one embodiment, the heterologous polynucleotide encoding the xylulokinase (XK) comprises the coding sequence of any xylulokinase described or referenced herein (e.g., the xylulokinase of SEQ ID NO: 75, 310 or 311). In one embodiment, the heterologous polynucleotide encoding the xylulokinase comprises a subsequence of the coding sequence from any xylulokinase described or referenced herein, wherein the subsequence encodes a polypeptide having xylulokinase activity. In one embodiment, the number of nucleotides residues in the subsequence is at least 75%, e.g., at least 80%, 85%, 90%, or 95% of the number of the referenced coding sequence.

The xylulokinases (XKs) can also include fused polypeptides or cleavable fusion polypeptides, as described supra.

In some embodiments, the host cells or fermenting organisms described herein have an active arabinose fermentation pathway known as the “bacterial pathway” shown in FIG. 1 which utilizes genes L-arabinose isomerase (AI, such as araA), L-ribulokinase (RK, such as araB), and L-ribulose-5-P4-epimerase (R5PE, such as araD) to convert L-arabinose to D-xylulose 5-phosphate. This and other exemplary arabinose fermentation pathways are known in the art (e.g., WO2002/066616; WO2003/095627; WO2007/143245; WO2008/041840; WO2009/011591; WO2010/151548; WO2011/003893; WO2011/131674; WO2012/143513; US2012/225464; U.S. Pat. No. 7,977,083). Any arabinose fermentation pathway or gene thereof described in the foregoing references is incorporated herein by reference for use in Applicant's active arabinose fermentation pathway.

In one aspect, the recombinant cells described herein (e.g., a cell comprising a heterologous polynucleotide encoding a L-xylulose reductase described herein) have improved anaerobic growth on a pentose (e.g., xylose and/or arabinose). In one embodiment, the recombinant cell is capable of higher anaerobic growth rate on a pentose (e.g., xylose and/or arabinose) compared to the same cell without the heterologous polynucleotide encoding L-xylulose reductase (e.g., under conditions described in the examples herein). In one embodiment, the recombinant cell is capable of higher anaerobic growth rate on pentose (e.g., xylose and/or arabinose) compared to the same cell encoding the L-xylulose reductase (LXR) of SEQ ID NO: 305 (e.g., under conditions described in the examples herein).

In one aspect, the recombinant cells described herein (e.g., a cell comprising a heterologous polynucleotide encoding a L-xylulose reductase described herein) have improved rate of pentose consumption (e.g., xylose and/or arabinose). In one embodiment, the recombinant cell is capable of higher rate of pentose consumption (e.g., xylose and/or arabinose) compared to the same cell without the heterologous polynucleotide encoding a L-xylulose reductase (e.g., under conditions described in the examples herein). In one embodiment, the rate of pentose consumption (e.g., xylose and/or arabinose) is at least 5%, e.g., at least 10%, 15%, 20%, 25%, 30%, 35%, 40%, 45%, 50%, 60%, 75% or 90% higher compared to the same cell without the heterologous polynucleotide encoding a L-xylulose reductase (e.g., under conditions described in the examples herein). In one embodiment, the cell is capable of a higher rate of pentose consumption (e.g., at least 5%, 10%, 15%, 20%, 25%, 30%, 35%, 40%, 45%, 50%, 60%, 75% or 90% higher xylose and/or arabinose consumption) compared to the same cell encoding the L-xylulose reductase (LXR) of SEQ ID NO: 305 (e.g., under conditions described in the examples herein).

In one aspect, the recombinant cells described herein (e.g., a cell comprising a heterologous polynucleotide encoding a L-xylulose reductase described herein) have higher pentose (e.g., xylose and/or arabinose) consumption. In one embodiment, the recombinant cell is capable of higher pentose (e.g., xylose and/or arabinose) consumption compared to the same cell without the heterologous polynucleotide encoding a L-xylulose reductase at about or after 120 hours fermentation (e.g., under conditions described in the examples herein). In one embodiment, the recombinant cell is capable of consuming more than 65%, e.g., at least 70%, 75%, 80%, 85%, 90%, 95% of pentose (e.g., xylose and/or arabinose) in the medium at about or after 120 hours fermentation (e.g., under conditions described in the examples herein). In one embodiment, the recombinant cell is capable of higher pentose consumption (e.g., xylose and/or arabinose) compared to the same cell encoding the L-xylulose reductase (LXR) of SEQ ID NO: 305 at about or after 120 hours fermentation (e.g., under conditions described in the examples herein).

### Active Xylose Fermentation Pathway

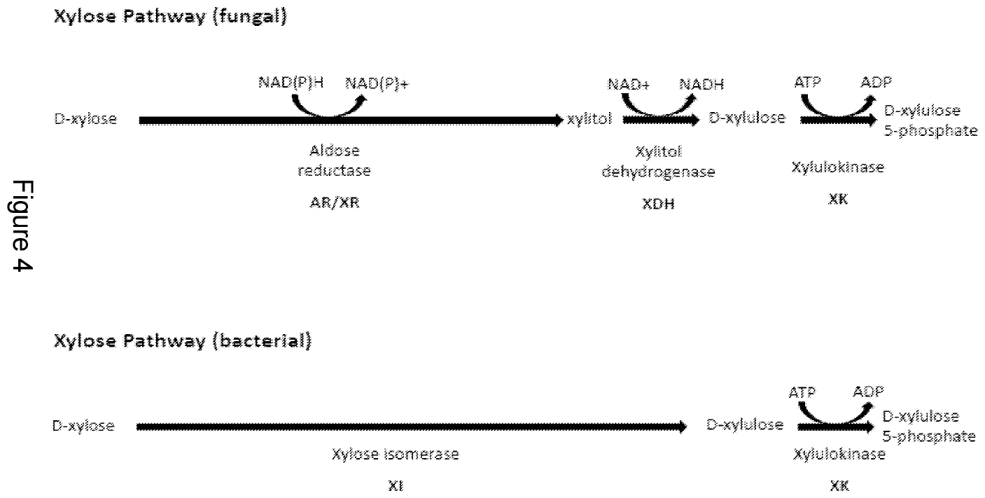

The host cells or fermenting organisms described herein (e.g., yeast cells) may comprise an active xylose fermentation pathway. Exemplary xylose fermentation pathways are known in the art (e.g., WO2003/062430, WO2003/078643, WO2004/067760, WO02006/096130, WO02009/017441, WO02010/059095, WO2011/059329, WO02011/123715, WO02012/113120, WO02012/135110, WO2013/081700, WO02018/112638 and US2017/088866). Any xylose fermentation pathway or gene thereof described in the foregoing references is incorporated herein by reference for use in Applicant's active xylose fermentation pathway. FIG. 4 shows conversion of D-xylose to D-xylulose 5-phosphate, which is then fermented to ethanol via the pentose phosphate pathway. The oxido-reductase pathway uses an aldolase reductase (AR, such as xylose reductase (XR)) to reduce D-xylose to xylitol followed by oxidation of xylitol to D-xylulose with xylitol dehydrogenase (XDH; also known as D-xylulose reductase). The isomerase pathway uses xylose isomerase (XI) to convert D-xylose into D-xylulose. D-xylulose is then converted to D-xylulose-5-phosphate with xylulokinase (XK). The active xylose fermentation pathway may utilize one or more of the same enzymes of an active arabinose fermentation pathway (e.g., AR, XDH, XK).

In one embodiment, the host cell or fermenting organism (e.g., yeast cell) further comprises a heterologous polynucleotide encoding a xylose isomerase (XI). The xylose isomerase may be any xylose isomerase that is suitable for the host cells and the methods described herein, such as a naturally occurring xylose isomerase or a variant thereof that retains xylose isomerase activity. In one embodiment, the xylose isomerase is present in the cytosol of the host cells.

In some embodiments, the host cell or fermenting organism comprising a heterologous polynucleotide encoding a xylose isomerase has an increased level of xylose isomerase activity compared to the host cells without the heterologous polynucleotide encoding the xylose isomerase, when cultivated under the same conditions. In some embodiments, the host cells or fermenting organisms have an increased level of xylose isomerase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding the xylose isomerase, when cultivated under the same conditions.

Exemplary xylose isomerases that can be used with the recombinant host cells and methods of use described herein include, but are not limited to, XIs from the fungus Piromyces sp. (WO2003/062430) or other sources (Madhavan et al., 2009, Appl Microbiol Biotechnol. 82(6), 1067-1078) have been expressed in S. cerevisiae host cells. Still other XIs suitable for expression in yeast have been described in US 2012/0184020 (an XI from Ruminococcus flavefaciens), WO02011/078262 (several XIs from Reticulitermes speratus and Mastotermes darwiniensis) and WO02012/009272 (constructs and fungal cells containing an XI from Abiotrophia defectiva). U.S. Pat. No. 8,586,336 describes a S. cerevisiae host cell expressing an XI obtained by bovine rumen fluid (shown herein as SEQ ID NO: 74).

Additional polynucleotides encoding suitable xylose isomerases may be obtained from microorganisms of any genus, including those readily available within the UniProtKB database. In one embodiment, the xylose isomerase is a bacterial, a yeast, or a filamentous fungal xylose isomerase, e.g., obtained from any of the microorganisms described or referenced herein, as described supra.

The xylose isomerase coding sequences can also be used to design nucleic acid probes to identify and clone DNA encoding xylose isomerases from strains of different genera or species, as described supra.

The polynucleotides encoding xylose isomerases may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding xylose isomerases are described supra.

In one embodiment, the xylose isomerase has a mature polypeptide sequence of having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any xylose isomerase described or referenced herein (e.g., the xylose isomerase of SEQ ID NO: 74). In one embodiment, the xylose isomerase has a mature polypeptide sequence that differs by no more than ten amino acids, e.g., by no more than five amino acids, by no more than four amino acids, by no more than three amino acids, by no more than two amino acids, or by one amino acid from any xylose isomerase described or referenced herein (e.g., the xylose isomerase of SEQ ID NO: 74). In one embodiment, the xylose isomerase has a mature polypeptide sequence that comprises or consists of the amino acid sequence of any xylose isomerase described or referenced herein (e.g., the xylose isomerase of SEQ ID NO: 74), allelic variant, or a fragment thereof having xylose isomerase activity. In one embodiment, the xylose isomerase has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the xylose isomerase has at least 20%, e.g., at least 40%, at least 50%, at least 60%, at least 70%, at least 80%, at least 90%, at least 95%, at least 96%, at least 97%, at least 98%, at least 99%, or 100% of the xylose isomerase activity of any xylose isomerase described or referenced herein (e.g., the xylose isomerase of SEQ ID NO: 74) under the same conditions.

In one embodiment, the xylose isomerase coding sequence hybridizes under at least low stringency conditions, e.g., medium stringency conditions, medium-high stringency conditions, high stringency conditions, or very high stringency conditions with the full-length complementary strand of the coding sequence from any xylose isomerase described or referenced herein (e.g., the xylose isomerase of SEQ ID NO: 74). In one embodiment, the xylose isomerase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any xylose isomerase described or referenced herein (e.g., the xylose isomerase of SEQ ID NO: 74).

In one embodiment, the heterologous polynucleotide encoding the xylose isomerase comprises the coding sequence of any xylose isomerase described or referenced herein (e.g., the xylose isomerase of SEQ ID NO: 74). In one embodiment, the heterologous polynucleotide encoding the xylose isomerase comprises a subsequence of the coding sequence from any xylose isomerase described or referenced herein, wherein the subsequence encodes a polypeptide having xylose isomerase activity. In one embodiment, the number of nucleotides residues in the subsequence is at least 75%, e.g., at least 80%, 85%, 90%, or 95% of the number of the referenced coding sequence.

The xylose isomerases can also include fused polypeptides or cleavable fusion polypeptides, as described supra.

The host cell or fermenting organism may also comprise an aldose reductase (AR), xylitol dehydrogenase (XDH) and/or xylulokinase (XK) as described supra.

In one embodiment, the host cell or fermenting organism (e.g., yeast cell) further comprises a heterologous polynucleotide encoding a ribulose 5 phosphate 3-epimerase (RPE1). A ribulose 5 phosphate 3-epimerase, as used herein, provides enzymatic activity for converting L-ribulose 5-phosphate to L-xylulose 5-phosphate (EC 5.1.3.22). The RPE1 may be any RPE1 that is suitable for the host cells and the methods described herein, such as a naturally occurring RPE1 or a variant thereof that retains RPE1 activity. In one embodiment, the RPE1 is present in the cytosol of the host cells.

In one embodiment, the recombinant cell comprises a heterologous polynucleotide encoding a ribulose 5 phosphate 3-epimerase (RPE1), wherein the RPE1 is Saccharomyces cerevisiae RPE1, or an RPE1 having at least 60%, e.g., at least 65%, 70%, 75%, 80%, 85%, 90%, 95%, 97%, 98%, 99%, or 100% sequence identity to a Saccharomyces cerevisiae RPE1.

In one embodiment, the host cell or fermenting organism (e.g., yeast cell) further comprises a heterologous polynucleotide encoding a ribulose 5 phosphate isomerase (RKI1).

A ribulose 5 phosphate isomerase, as used herein, provides enzymatic activity for converting ribose-5-phophate to ribulose 5-phosphate. The RKI1 may be any RKI1 that is suitable for the host cells and the methods described herein, such as a naturally occurring RKI1 or a variant thereof that retains RKI1 activity. In one embodiment, the RKI1 is present in the cytosol of the host cells.

In one embodiment, the host cell or fermenting organism comprises a heterologous polynucleotide encoding a ribulose 5 phosphate isomerase (RKI1), wherein the RKI1 is a Saccharomyces cerevisiae RKI1, or an RKI1 having a mature polypeptide sequence of at least 60%, e.g., at least 65%, 70%, 75%, 80%, 85%, 90%, 95%, 97%, 98%, 99%, or 100% sequence identity to a Saccharomyces cerevisiae RKI1.

In one embodiment, the host cell or fermenting organism (e.g., yeast cell) further comprises a heterologous polynucleotide encoding a transketolase (TKL1). The TKL1 may be any TKL1 that is suitable for the host cells and the methods described herein, such as a naturally occurring TKL1 or a variant thereof that retains TKL1 activity. In one embodiment, the TKL1 is present in the cytosol of the host cells.

In one embodiment, the host cell or fermenting organism comprises a heterologous polynucleotide encoding a transketolase (TKL1), wherein the TKL1 is a Saccharomyces cerevisiae TKL1, or a TKL1 having a mature polypeptide sequence of at least 60%, e.g., at least 65%, 70%, 75%, 80%, 85%, 90%, 95%, 97%, 98%, 99%, or 100% sequence identity to a Saccharomyces cerevisiae TKL1.

In one embodiment, the host cell or fermenting organism (e.g., yeast cell) further comprises a heterologous polynucleotide encoding a transaldolase (TAL1). The TAL1 may be any TAL1 that is suitable for the host cells and the methods described herein, such as a naturally occurring TAL1 or a variant thereof that retains TAL1 activity. In one embodiment, the TAL1 is present in the cytosol of the host cells.

In one embodiment, the host cell or fermenting organism comprises a heterologous polynucleotide encoding a transketolase (TAL1), wherein the TAL1 is a Saccharomyces cerevisiae TAL1, or a TAL1 having a mature polypeptide sequence of at least 60%, e.g., at least 65%, 70%, 75%, 80%, 85%, 90%, 95%, 97%, 98%, 99%, or 100% sequence identity to a Saccharomyces cerevisiae TAL1.

### Non-Phosphorylating NADP-Dependent Glyceraldehyde-3-Phosphate Dehydrogenases (GAPNs)

The host cells and fermenting organisms may express a heterologous NADP-dependent glyceraldehyde-3-phosphate dehydrogenase (GAPN). The GAPN can be any GAPN that is suitable for the host cells and their methods of use described herein, such as a naturally occurring GAPN (e.g., an endogenous GAPN or a native GAPN from another species) or a variant thereof that retains GAPN activity. In one aspect, GAPN is present in the cytosol of the host cells.

In some embodiments, the host cell or fermenting organism comprises a heterologous polynucleotide encoding a GAPN. In some embodiments, the host cell or fermenting organism comprising a heterologous polynucleotide encoding a GAPN has an increased level of GAPN activity compared to the host cell or fermenting organism without the heterologous polynucleotide encoding the GAPN, when cultivated under the same conditions. In some embodiments, the host cell or fermenting organism has an increased level of GAPN activity of at least 5%, e.g., at least 10%, at least 15%, at least 20%, at least 25%, at least 50%, at least 100%, at least 150%, at least 200%, at least 300%, or at 500% compared to host cell or fermenting organism without the heterologous polynucleotide encoding the GAPN, when cultivated under the same conditions.

Exemplary GAPNs that may be expressed with the host cells or fermenting organisms and methods of use described herein include, but are not limited to, GAPNs shown in Table 3 (or derivatives thereof).

TABLE 3

| | Donor Organism | Sequence code | SEQ ID NO. |
|---|---|---|---|
| 1 | Triticum aestivum | Q8LK61 | 262 |
| 2 | Chlamydomonas reinhardtii | A0A2K3D5S6 | 263 |
| 3 | Apium graveolens | Q9SNX8 | 264 |
| 4 | Cicer arietinum | A0A1S2YP36 | 265 |
| 5 | Bacillus pseudomycoides | A0A2C4I5G8 | 266 |
| 6 | Streptococcus equinus | Q3C1A6 | 267 |
| 7 | Glycine soja | A0A0B2QEZ3 | 268 |
| 8 | Streptococcus sp. DD12 | A0A139NKR4 | 269 |
| 9 | Bacillus thuringiensis | A0A0B5NZK7 | 270 |
| 10 | Arabidopsis thaliana | Q1WIQ6 | 271 |
| 11 | Bacillus litoralis | EFP8C9GVR | 272 |
| 12 | Streptococcus hyointestinalis | A0A380K8A8 | 273 |
| 13 | Zea mays | Q43272 | 274 |
| 14 | Lactobacillus delbrueckii | Q04A83 | 275 |
| 15 | Streptococcus pluranimalium | A0A2L0D390 | 276 |
| 16 | Nicotiana plumbaginifolia | P93338 | 277 |
| 17 | Streptococcus macacae | G5JUQ8 | 278 |
| 18 | Streptococcus mutans | Q59931 | 279 |
| 19 | Bacillus cereus | | 280 |

Additional polynucleotides encoding suitable GAPNs may be derived from microorganisms of any suitable genus, including those readily available within the UniProtKB database.

The GAPN coding sequences can also be used to design nucleic acid probes to identify and clone DNA encoding proteases from strains of different genera or species, as described supra.

The polynucleotides encoding GAPNs may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding GAPNs are described supra.

In one embodiment, the GAPN has a mature polypeptide sequence that comprises or consists of the amino acid sequence of any one of SEQ ID NOs: 262-280 (e.g., any one of SEQ ID NOs: 262, 263, 264, 265, 266, 267, 268, 269, 270, 271, 272, 273, 274, 275, 276, 277, 278, 279 or 280). In another embodiment, the GAPN has a mature polypeptide sequence that is a fragment of the GAPN of any one of SEQ ID NOs: 262-280 (e.g., wherein the fragment has GAPN activity). In one embodiment, the number of amino acid residues in the fragment is at least 75%, e.g., at least 80%, 85%, 90%, or 95% of the number of amino acid residues in referenced full length GAPN (e.g. any one of SEQ ID NOs: 262-280). In other embodiments, the GAPN may comprise the catalytic domain of any GAPN described or referenced herein (e.g., the catalytic domain of any one of SEQ ID NOs: 262-280).

The GAPN may be a variant of any one of the GAPNs described supra (e.g., any one of SEQ ID NOs: 262-280). In one embodiment, the GAPN has a mature polypeptide sequence of at least 60%, e.g., at least 65%, 70%, 75%, 80%, 85%, 90%, 95%, 97%, 98%, 99%, or 100% sequence identity to any one of the GAPNs described supra (e.g., any one of SEQ ID NOs: 262-280).

Examples of suitable amino acid changes, such as conservative substitutions that do not significantly affect the folding and/or activity of the GAPN, are described supra.

In one embodiment, the GAPN has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the GAPN described supra (e.g., any one of SEQ ID NOs: 262-280). In one embodiment, the GAPN has an amino acid substitution, deletion, and/or insertion of one or more (e.g., two, several) of amino acid sequence of any one of the GAPNs described supra (e.g., any one of SEQ ID NOs: 262-280). In some embodiments, the total number of amino acid substitutions, deletions and/or insertions is not more than 10, e.g., not more than 9, 8, 7, 6, 5, 4, 3, 2, or 1.

In one embodiment, the GAPN coding sequence hybridizes under at least low stringency conditions, e.g., medium stringency conditions, medium-high stringency conditions, high stringency conditions, or very high stringency conditions with the full-length complementary strand of the coding sequence from any GAPN described or referenced herein (e.g., any one of SEQ ID NOs: 262-280). In one embodiment, the GAPN coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any GAPN described or referenced herein (e.g., any one of SEQ ID NOs: 262-280).

In one embodiment, the GAPN comprises the coding sequence of any GAPN described or referenced herein (any one of SEQ ID NOs: 262-280). In one embodiment, the GAPN comprises a coding sequence that is a subsequence of the coding sequence from any GAPN described or referenced herein, wherein the subsequence encodes a polypeptide having GAPN activity. In one embodiment, the number of nucleotides residues in the subsequence is at least 75%, e.g., at least 80%, 85%, 90%, or 95% of the number of the referenced coding sequence.

The referenced GAPN coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The GAPN can also include fused polypeptides or cleavable fusion polypeptides, as described supra.

### Glucoamylases

The host cells and fermenting organisms may express a heterologous glucoamylase. The glucoamylase can be any glucoamylase that is suitable for the host cells, fermenting organisms and/or their methods of use described herein, such as a naturally occurring glucoamylase or a variant thereof that retains glucoamylase activity. Any glucoamylase contemplated for expression by a host cell or fermenting organism described below is also contemplated for embodiments of the invention involving exogenous addition of a glucoamylase (e.g., added before, during or after liquefaction and/or saccharification).

In some embodiments, the host cell or fermenting organism comprises a heterologous polynucleotide encoding a glucoamylase, for example, as described in WO2017/087330, the content of which is hereby incorporated by reference. Any glucoamylase described or referenced herein is contemplated for expression in the host cell or fermenting organism.

In some embodiments, the host cell or fermenting organism comprising a heterologous polynucleotide encoding a glucoamylase has an increased level of glucoamylase activity compared to the host cells without the heterologous polynucleotide encoding the glucoamylase, when cultivated under the same conditions. In some embodiments, the host cell or fermenting organism has an increased level of glucoamylase activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the glucoamylase, when cultivated under the same conditions.

Exemplary glucoamylases that can be used with the host cells and/or the methods described herein include bacterial, yeast, or filamentous fungal glucoamylases, e.g., obtained from any of the microorganisms described or referenced herein, as described supra.

Preferred glucoamylases are of fungal or bacterial origin, selected from the group consisting of Aspergillus glucoamylases, in particular Aspergillus niger G1 or G2 glucoamylase (Boel et al. (1984), EMBO J. 3 (5), p. 1097-1102), or variants thereof, such as those disclosed in VO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. (1991), 55 (4), p. 941-949), or variants or fragments thereof. Other Aspergillus glucoamylase variants include variants with enhanced thermal stability: G137A and G139A (Chen et al. (1996), Prot. Eng. 9, 499-505); D257E and D293E/Q (Chen et al. (1995), Prot. Eng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Eng. 10, 1199-1204.

Other glucoamylases include Athelia rolfsii (previously denoted Corticium rolfsii) glucoamylase (see U.S. Pat. No. 4,727,026 and (Nagasaka et al. (1998) “Purification and properties of the raw-starch-degrading glucoamylases from Corticium rolfsii, Appl Microbiol Biotechnol 50:323-330), Talaromyces glucoamylases, in particular derived from Talaromyces emersonii(WO 99/28448), Talaromyces leycettanus (US patent no. Re. 32,153), Talaromyces duponti, Talaromyces thermophilus (U.S. Pat. No. 4,587,215). In one embodiment, the glucoamylase used during saccharification and/or fermentation is the Talaromyces emersonii glucoamylase disclosed in WO 99/28448 or the Talaromyces emersonii glucoamylase of SEQ ID NO: 247.

Bacterial glucoamylases contemplated include glucoamylases from the genus Clostridium, in particular C. thermoamylolyticum (EP 135,138), and C. thermohydiosulfuricum (WO 86/01831).

Contemplated fungal glucoamylases include Trametes cingulate, Pachykytospora papyracea; and Leucopaxillus giganteus all disclosed in WO2006/069289; or Peniophora rufomarginata disclosed in WO2007/124285; or a mixture thereof. Also hybrid glucoamylase are contemplated. Examples include the hybrid glucoamylases disclosed in WO2005/045018.

In one embodiment, the glucoamylase is derived from a strain of the genus Pycnoporus, in particular a strain of Pycnoporus as described in WO2011/066576 (SEQ ID NO: 2, 4 or 6 therein), including the Pycnoporus sanguineus glucoamylase, or from a strain of the genus Gloeophyllum, such as a strain of Gloeophyllum sepiarium or Gloeophyllum trabeum, in particular a strain of Gloeophyllum as described in WO2011/068803 (SEQ ID NO: 2, 4, 6, 8, 10, 12, 14 or 16 therein). In one embodiment, the glucoamylase is SEQ ID NO: 2 in WO02011/068803 (i.e. Gloeophyllum sepiarium glucoamylase). In one embodiment, the glucoamylase is the Gloeophyllum sepiarium glucoamylase of SEQ ID NO: 8. In one embodiment, the glucoamylase is the Pycnoporus sanguineus glucoamylase of SEQ ID NO: 229.

In one embodiment, the glucoamylase is a Gloeophyllum trabeum glucoamylase (disclosed as SEQ ID NO: 3 in WO2014/177546). In another embodiment, the glucoamylase is derived from a strain of the genus Nigrofomes, in particular a strain of Nigrofomes sp. disclosed in WO02012/064351 (disclosed as SEQ ID NO: 2 therein).

Also contemplated are glucoamylases with a mature polypeptide sequence which exhibit a high identity to any of the above mentioned glucoamylases, i.e., at least 60%, such as at least 70%, at least 75%, at least 80%, at least 85%, at least 90%, at least 95%, at least 96%, at least 97%, at least 98%, at least 99% or even 100% identity to any one of the mature polypeptide sequences mentioned above.

Glucoamylases may be added to the saccharification and/or fermentation in an amount of 0.0001-20 AGU/g DS, such as 0.001-10 AGU/g DS, 0.01-5 AGU/g DS, or 0.1-2 AGU/g DS.

Glucoamylases may be added to the saccharification and/or fermentation in an amount of 1-1,000 μg EP/g DS, such as 10-500 μg/gDS, or 25-250 μg/g DS.

Glucoamylases may be added to liquefaction in an amount of 0.1-100 μg EP/g DS, such as 0.5-50 μg EP/g DS, 1-25 μg EP/g DS, or 2-12 μg EP/g DS.

In one embodiment, the glucoamylase is added as a blend further comprising an alpha-amylase (e.g., any alpha-amylase described herein). In one embodiment, the alpha-amylase is a fungal alpha-amylase, especially an acid fungal alpha-amylase. The alpha-amylase is typically a side activity.

In one embodiment, the glucoamylase is a blend comprising Talaromyces emersonii glucoamylase disclosed in WO 99/28448 as SEQ ID NO: 34 and Trametes cingulata glucoamylase disclosed as SEQ ID NO: 2 in WO 06/069289.

In one embodiment, the glucoamylase is a blend comprising Talaromyces emersonii glucoamylase disclosed in WO 99/28448, Trametes cingulata glucoamylase disclosed as SEQ ID NO: 2 in WO 06/69289, and an alpha-amylase.

In one embodiment, the glucoamylase is a blend comprising Talaromyces emersonii glucoamylase disclosed in WO99/28448, Trametes cingulata glucoamylase disclosed in WO 06/69289, and Rhizomucor pusillus alpha-amylase with Aspergillus niger glucoamylase linker and SBD disclosed as V039 in Table 5 in WO2006/069290.

In one embodiment, the glucoamylase is a blend comprising Gloeophyllum sepiarium glucoamylase shown as SEQ ID NO: 2 in WO2011/068803 and an alpha-amylase, in particular Rhizomucor pusillus alpha-amylase with an Aspergillus niger glucoamylase linker and starch-binding domain (SBD), disclosed SEQ ID NO: 3 in WO2013/006756, in particular with the following substitutions: G128D+D143N.

In one embodiment, the alpha-amylase may be derived from a strain of the genus Rhizomucor, preferably a strain the Rhizomucor pusillus, such as the one shown in SEQ ID NO: 3 in WO2013/006756, or the genus Meripilus, preferably a strain of Meripilus giganteus. In one embodiment, the alpha-amylase is derived from a Rhizomucor pusillus with an Aspergillus niger glucoamylase linker and starch-binding domain (SBD), disclosed as V039 in Table 5 in WO2006/069290.

In one embodiment, the Rhizomucor pusillus alpha-amylase or the Rhizomucor pusillus alpha-amylase with an Aspergillus niger glucoamylase linker and starch-binding domain (SBD) has at least one of the following substitutions or combinations of substitutions: D165M; Y141W; Y141R; K136F; K192R; P224A; P224R; S123H+Y141W; G20S+Y141W; A76G+Y141W; G128D+Y141W; G128D+D143N; P219C+Y141W; N142D+D143N; Y141W+K192R; Y141W+D143N; Y141W+N383R; Y141W+P219C+A265C; Y141W+N142D+D143N; Y141W+K192R V410A; G128D+Y141W+D143N; Y141W+D143N+P219C; Y141W+D143N+K192R; G128D+D143N+K192R; Y141W+D143N+K192R+P219C; and G128D+Y141W+D143N+K192R; or G128D+Y141W+D143N+K192R+P219C (using SEQ ID NO: 3 in WO2013/006756 for numbering).

In one embodiment, the glucoamylase blend comprises Gloeophyllum sepiarium glucoamylase (e.g., SEQ ID NO: 2 in WO2011/068803) and Rhizomucor pusillus alpha-amylase.

In one embodiment, the glucoamylase blend comprises Gloeophyllum sepiarium glucoamylase shown as SEQ ID NO: 2 in WO2011/068803 and Rhizomucor pusillus with an Aspergillus niger glucoamylase linker and starch-binding domain (SBD), disclosed SEQ ID NO: 3 in WO2013/006756 with the following substitutions: G128D+D143N.

Commercially available compositions comprising glucoamylase include AMG 200L; AMG 300 L; SAN™ SUPER, SAN™ EXTRA L, SPIRIZYME® PLUS, SPIRIZYME® FUEL, SPIRIZYME® B4U, SPIRIZYME® ULTRA, SPIRIZYME® EXCEL, SPIRIZYME ACHIEVED, and AMG® E (from Novozymes A/S); OPTIDEX™ 300, GC480, GC417 (from DuPont-Danisco); AMIGASE™ and AMIGASE™ PLUS (from DSM); G-ZYME™ G900, G-ZYME™ and G990 ZR (from DuPont-Danisco).

In one embodiment, the glucoamylase is derived from the Debaryomyces occidentalis glucoamylase of SEQ ID NO: 102. In one embodiment, the glucoamylase is derived from the Saccharomycopsis fibuligera glucoamylase of SEQ ID NO: 103. In one embodiment, the glucoamylase is derived from the Saccharomycopsis fibuligera glucoamylase of SEQ ID NO: 104. In one embodiment, the glucoamylase is derived from the Saccharomyces cerevisiae glucoamylase of SEQ ID NO: 105. In one embodiment, the glucoamylase is derived from the Aspergillus niger glucoamylase of SEQ ID NO: 106. In one embodiment, the glucoamylase is derived from the Aspergillus oryzae glucoamylase of SEQ ID NO: 107. In one embodiment, the glucoamylase is derived from the Rhizopus oryzae glucoamylase of SEQ ID NO: 108 or SEQ ID NO: 250. In one embodiment, the glucoamylase is derived from the Clostridium thermocellum glucoamylase of SEQ ID NO: 109. In one embodiment, the glucoamylase is derived from the Clostridium thermocellum glucoamylase of SEQ ID NO: 110. In one embodiment, the glucoamylase is derived from the Arxula adeninivorans glucoamylase of SEQ ID NO: 111. In one embodiment, the glucoamylase is derived from the Hormoconis resinae glucoamylase of SEQ ID NO: 112. In one embodiment, the glucoamylase is derived from the Aureobasidium pullulans glucoamylase of SEQ ID NO: 113. In one embodiment, the glucoamylase is derived from the Rhizopus microsporus glucoamylase of SEQ ID NO: 248. In one embodiment, the glucoamylase is derived from the Rhizopus delemar glucoamylase of SEQ ID NO: 249. In one embodiment, the glucoamylase is derived from the Punctularia strigosozonata glucoamylase of SEQ ID NO: 244. In one embodiment, the glucoamylase is derived from the Fibroporia radiculosa glucoamylase of SEQ ID NO: 245. In one embodiment, the glucoamylase is derived from the Wolfiporia cocos glucoamylase of SEQ ID NO: 246.

In one embodiment, the glucoamylase is a Trichoderma reesei glucoamylase, such as the Trichoderma reesei glucoamylase of SEQ ID NO: 230.

In one embodiment, the glucoamylase has a Relative Activity heat stability at 85° C. of at least 20%, at least 30%, or at least 35% determined as described in Example 4 of WO2018/098381 (heat stability).

In one embodiment, the glucoamylase has a relative activity pH optimum at pH 5.0 of at least 90%, e.g., at least 95%, at least 97%, or 100% determined as described in Example 4 of WO2018/098381 (pH optimum).

In one embodiment, the glucoamylase has a pH stability at pH 5.0 of at least 80%, at least 85%, at least 90% determined as described in Example 4 of WO2018/098381 (pH stability).

In one embodiment, the glucoamylase used in liquefaction, such as a Penicillium oxalicum glucoamylase variant, has a thermostability determined as DSC Td at pH 4.0 as described in Example 15 of WO2018/098381 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C. In one embodiment, the glucoamylase, such as a Penicillium oxalicum glucoamylase variant, has a thermostability determined as DSC Td at pH 4.0 as described in Example 15 of WO2018/098381 in the range between 70° C. and 95° C., such as between 80° C. and 90° C.

In one embodiment, the glucoamylase, such as a Penicillium oxalicum glucoamylase variant, used in liquefaction has a thermostability determined as DSC Td at pH 4.8 as described in Example 15 of WO2018/098381 of at least 70° C., preferably at least 75° C., such as at least 80° C., such as at least 81° C., such as at least 82° C., such as at least 83° C., such as at least 84° C., such as at least 85° C., such as at least 86° C., such as at least 87%, such as at least 88° C., such as at least 89° C., such as at least 90° C., such as at least 91° C. In one embodiment, the glucoamylase, such as a Penicillium oxalicum glucoamylase variant, has a thermostability determined as DSC Td at pH 4.8 as described in Example 15 of WO2018/098381 in the range between 70° C. and 95° C., such as between 80° C. and 90° C.

In one embodiment, the glucoamylase, such as a Penicillium oxalicum glucoamylase variant, used in liquefaction has a residual activity determined as described in Example 16 of WO2018/098381, of at least 100% such as at least 105%, such as at least 110%, such as at least 115%, such as at least 120%, such as at least 125%. In one embodiment, the glucoamylase, such as a Penicillium oxalicum glucoamylase variant, has a thermostability determined as residual activity as described in Example 16 of WO2018/098381, in the range between 100% and 130%.

In one embodiment, the glucoamylase, e.g., of fungal origin such as a filamentous fungi, from a strain of the genus Penicillium, e.g., a strain of Penicillium oxalicum, in particular the Penicillium oxalicum glucoamylase disclosed as SEQ ID NO: 2 in WO02011/127802 (which is hereby incorporated by reference).

In one embodiment, the glucoamylase has a mature polypeptide sequence of at least 80%, e.g., at least 85%, at least 90%, at least 91%, at least 92%, at least 93%, at least 94%, at least 95%, at least 96%, at least 97%, at least 98%, at least 99% or 100% identity to the mature polypeptide shown in SEQ ID NO: 2 in WO2011/127802.

In one embodiment, the glucoamylase is a variant of the Penicillium oxalicum glucoamylase disclosed as SEQ ID NO: 2 in WO2011/127802, having a K79V substitution.

The K79V glucoamylase variant has reduced sensitivity to protease degradation relative to the parent as disclosed in WO02013/036526 (which is hereby incorporated by reference).

In one embodiment, the glucoamylase is derived from Penicillium oxalicum.

In one embodiment, the glucoamylase is a variant of the Penicillium oxalicum glucoamylase disclosed as SEQ ID NO: 2 in WO2011/127802. In one embodiment, the Penicillium oxalicum glucoamylase is the one disclosed as SEQ ID NO: 2 in WO02011/127802 having Val (V) in position 79.

Contemplated Penicillium oxalicum glucoamylase variants are disclosed in WO02013/053801 which is hereby incorporated by reference.

In one embodiment, these variants have reduced sensitivity to protease degradation.

In one embodiment, these variants have improved thermostability compared to the parent.

In one embodiment, the glucoamylase has a K79V substitution (using SEQ ID NO: 2 of WO02011/127802 for numbering), corresponding to the PE001 variant, and further comprises one of the following alterations or combinations of alterations T65A; Q327F; E501V; Y504T; Y504*; T65A+Q327F; T65A+E501V; T65A+Y504T; T65A+Y504*; Q327F+E501V; Q327F+Y504T; Q327F+Y504*; E501V+Y504T; E501V+Y504*; T65A+Q327F+E501V; T65A+Q327F+Y504T; T65A+E501V+Y504T; Q327F+E501V+Y504T; T65A+Q327F+Y504*; T65A+E501V+Y504*; Q327F+E501V+Y504*; T65A+Q327F+E501V+Y504T; T65A+Q327F+E501V+Y504*; E501V+Y504T; T65A+K161S; T65A+Q405T; T65A+Q327W; T65A+Q327F; T65A+Q327Y; P11F+T65A+Q327F; R1K+D3W+K5Q+G7V+N8S+T10K+P11S+T65A+Q327F; P2N+P4S+P11F+T65A+Q327F; P11F+D26C+K33C+T65A+Q327F; P2N+P4S+P11F+T65A+Q327W+E501V+Y504T; R1E+D3N+P4G+G6R+G7A+N8A+T10D+P11D+T65A+Q327F; P11F+T65A+Q327W; P2N+P4S+P11F+T65A+Q327F+E501V+Y504T; P11F+T65A+Q327W+E501V+Y504T; T65A+Q327F+E501V+Y504T; T65A+S105P+Q327W; T65A+S105P+Q327F; T65A+Q327W+S364P; T65A+Q327F+S364P; T65A+S103N+Q327F; P2N+P4S+P11F+K34Y+T65A+Q327F; P2N+P4S+P11F+T65A+Q327F+D445N+V447S; P2N+P4S+P11F+T65A+1172V+Q327F; P2N+P4S+P11F+T65A+Q327F+N502*; P2N+P4S+P11F+T65A+Q327F+N502T+P563S+K571E; P2N+P4S+P11F+R31S+K33V+T65A+Q327F+N564D+K571S; P2N+P4S+P11F+T65A+Q327F+S377T; P2N+P4S+P11F+T65A+V325T+Q327W; P2N+P4S+P11F+T65A+Q327F+D445N+V447S+E501V+Y504T; P2N+P4S+P11F+T65A+1172V+Q327F+E501V+Y504T; P2N+P4S+P11F+T65A+Q327F+S377T+E501V+Y504T; P2N+P4S+P11F+D26N+K34Y+T65A+Q327F; P2N+P4S+P11F+T65A+Q327F+1375A+E501V+Y504T; P2N+P4S+P11F+T65A+K218A+K221D+Q327F+E501V+Y504T; P2N+P4S+P11F+T65A+S103N+Q327F+E501V+Y504T; P2N+P4S+T10D+T65A+Q327F+E501V+Y504T; P2N+P4S+F12Y+T65A+Q327F+E501V+Y504T; K5A+P11F+T65A+Q327F+E501V+Y504T; P2N+P4S+T10E+E18N+T65A+Q327F+E501V+Y504T; P2N+T10E+E18N+T65A+Q327F+E501V+Y504T; P2N+P4S+P11F+T65A+Q327F+E501V+Y504T+T568N; P2N+P4S+P11F+T65A+Q327F+E501V+Y504T+K524T+G526A; P2N+P4S+P11F+K34Y+T65A+Q327F+D445N+V447S+E501V+Y504T; P2N+P4S+P11F+R31S+K33V+T65A+Q327F+D445N+V447S+E501V+Y504T; P2N+P4S+P11F+D26N+K34Y+T65A+Q327F+E501V+Y504T; P2N+P4S+P11F+T65A+F80*+Q327F+E501V+Y504T; P2N+P4S+P11F+T65A+K112S+Q327F+E501V+Y504T; P2N+P4S+P11F+T65A+Q327F+E501V+Y504T+T516P+K524T+G526A; P2N+P4S+P11F+T65A+Q327F+E501V+N502T+Y504*; P2N+P4S+P11F+T65A+Q327F+E501V+Y504T; P2N+P4S+P11F+T65A+S103N+Q327F+E501V+Y504T; K5A+P11F+T65A+Q327F+E501V+Y504T; P2N+P4S+P11F+T65A+Q327F+E501V+Y504T+T516P+K524T+G526A; P2N+P4S+P11F+T65A+V79A+Q327F+E501V+Y504T; P2N+P4S+P11F+T65A+V79G+Q327F+E501V+Y504T; P2N+P4S+P11F+T65A+V791+Q327F+E501V+Y504T; P2N+P4S+P11F+T65A+V79L+Q327F+E501V+Y504T; P2N+P4S+P11F+T65A+V79S+Q327F+E501V+Y504T; P2N+P4S+P11F+T65A+L72V+Q327F+E501V+Y504T; S255N+Q327F+E501V+Y504T; P2N+P4S+P11F+T65A+E74N+V79K+Q327F+E501V+Y504T; P2N+P4S+P11F+T65A+G220N+Q327F+E501V+Y504T; P2N+P4S+P11F+T65A+Y245N+Q327F+E501V+Y504T; P2N+P4S+P11F+T65A+Q253N+Q327F+E501V+Y504T; P2N+P4S+P11F+T65A+D279N+Q327F+E501V+Y504T; P2N+P4S+P11F+T65A+Q327F+S359N+E501V+Y504T; P2N+P4S+P11F+T65A+Q327F+D370N+E501V+Y504T; P2N+P4S+P11F+T65A+Q327F+V460S+E501V+Y504T; P2N+P4S+P11F+T65A+Q327F+V460T+P468T+E501V+Y504T; P2N+P4S+P11F+T65A+Q327F+T463N+E501V+Y504T; P2N+P4S+P11F+T65A+Q327F+S465N+E501V+Y504T; and P2N+P4S+P11F+T65A+Q327F+T477N+E501V+Y504T.

In one embodiment, the Penicillium oxalicum glucoamylase variant has a K79V substitution (using SEQ ID NO: 2 of WO2011/127802 for numbering), corresponding to the PE001 variant, and further comprises one of the following substitutions or combinations of substitutions:

P11F+T65A+Q327F;

P2N+P4S+P11F+T65A+Q327F;

P11F+D26C+K33C+T65A+Q327F;

P2N+P4S+P11F+T65A+Q327W+E501V+Y504T;

P2N+P4S+P11F+T65A+Q327F+E501V+Y504T; and

P11F+T65A+Q327W+E501V+Y504T.

Additional glucoamylases contemplated for use with the present invention can be found in WO02011/153516 (the content of which is incorporated herein).

Additional polynucleotides encoding suitable glucoamylases may be obtained from microorganisms of any genus, including those readily available within the UniProtKB database.

The glucoamylase coding sequences can also be used to design nucleic acid probes to identify and clone DNA encoding glucoamylases from strains of different genera or species, as described supra.

The polynucleotides encoding glucoamylases may also be identified and obtained from other sources including microorganisms isolated from nature (e.g., soil, composts, water, etc.) or DNA samples obtained directly from natural materials (e.g., soil, composts, water, etc) as described supra.

Techniques used to isolate or clone polynucleotides encoding glucoamylases are described supra.

In one embodiment, the glucoamylase has a mature polypeptide sequence that comprises or consists of the amino acid sequence of any one of the glucoamylases described or referenced herein (e.g., any one of SEQ ID NOs: 8, 102-113, 229, 230 and 244-250). In another embodiment, the glucoamylase has a mature polypeptide sequence that is a fragment of the any one of the glucoamylases described or referenced herein (e.g., any one of SEQ ID NOs: 8, 102-113, 229, 230 and 244-250). In one embodiment, the number of amino acid residues in the fragment is at least 75%, e.g., at least 80%, 85%, 90%, or 95% of the number of amino acid residues in referenced full length glucoamylase (e.g. any one of SEQ ID NOs: 8, 102-113, 229, 230 and 244-250). In other embodiments, the glucoamylase may comprise the catalytic domain of any glucoamylase described or referenced herein (e.g., the catalytic domain of any one of SEQ ID NOs: 8, 102-113, 229, 230 and 244-250).

The glucoamylase may be a variant of any one of the glucoamylases described supra (e.g., any one of SEQ ID NOs: 8,102-113, 229, 230 and 244-250). In one embodiment, the glucoamylase has a mature polypeptide sequence of at least 60%, e.g., at least 65%, 70%, 75%, 80%, 85%, 90%, 95%, 97%, 98%, 99%, or 100% sequence identity to any one of the glucoamylases described supra (e.g., any one of SEQ ID NOs: 8, 102-113, 229, 230 and 244-250).

Examples of suitable amino acid changes, such as conservative substitutions that do not significantly affect the folding and/or activity of the glucoamylase, are described herein.

In one embodiment, the glucoamylase has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the glucoamylases described supra (e.g., any one of SEQ ID NOs: 8, 102-113, 229, 230 and 244-250). In one embodiment, the glucoamylase has an amino acid substitution, deletion, and/or insertion of one or more (e.g., two, several) of amino acid sequence of any one of the glucoamylases described supra (e.g., any one of SEQ ID NOs: 8,102-113,229, 230 and 244-250). In some embodiments, the total number of amino acid substitutions, deletions and/or insertions is not more than 10, e.g., not more than 9, 8, 7, 6, 5, 4, 3, 2, or 1.

In some embodiments, the glucoamylase has at least 20%, e.g., at least 40%, at least 50%, at least 60%, at least 70%, at least 80%, at least 90%, at least 95%, at least 96%, at least 97%, at least 98%, at least 99%, or 100% of the glucoamylase activity of any glucoamylase described or referenced herein (e.g., any one of SEQ ID NOs: 8, 102-113, 229, 230 and 244-250) under the same conditions.

In one embodiment, the glucoamylase coding sequence hybridizes under at least low stringency conditions, e.g., medium stringency conditions, medium-high stringency conditions, high stringency conditions, or very high stringency conditions with the full-length complementary strand of the coding sequence from any glucoamylase described or referenced herein (e.g., any one of SEQ ID NOs: 8, 102-113, 229, 230 and 244-250). In one embodiment, the glucoamylase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any glucoamylase described or referenced herein (e.g., any one of SEQ ID NOs: 8, 102-113, 229, 230 and 244-250).

In one embodiment, the glucoamylase comprises the coding sequence of any glucoamylase described or referenced herein (any one of SEQ ID NOs: 8, 102-113, 229, 230 and 244-250). In one embodiment, the glucoamylase comprises a coding sequence that is a subsequence of the coding sequence from any glucoamylase described or referenced herein, wherein the subsequence encodes a polypeptide having glucoamylase activity. In one embodiment, the number of nucleotides residues in the subsequence is at least 75%, e.g., at least 80%, 85%, 90%, or 95% of the number of the referenced coding sequence.

The referenced glucoamylase coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The glucoamylase can also include fused polypeptides or cleavable fusion polypeptides, as described supra. In some embodiments, the glucoamylase is a fusion protein comprising a signal peptide linked to the N-terminus of a mature polypeptide, such as any signal sequences described in U.S. Provisional Application No. 62/883,519 filed Aug. 6, 2019 and entitled “Fusion Proteins For Improved Enzyme Expression” (the content of which is hereby incorporated by reference).

### Alpha-Amylases

The host cells and fermenting organisms may express a heterologous alpha-amylase. The alpha-amylase may be any alpha-amylase that is suitable for the host cells and/or the methods described herein, such as a naturally occurring alpha-amylase (e.g., a native alpha-amylase from another species or an endogenous alpha-amylase expressed from a modified expression vector) or a variant thereof that retains alpha-amylase activity. Any alpha-amylase contemplated for expression by a host cell or fermenting organism described below is also contemplated for embodiments of the invention involving exogenous addition of an alpha-amylase.

In some embodiments, the host cell or fermenting organism comprises a heterologous polynucleotide encoding an alpha-amylase, for example, as described in WO02017/087330 or WO02020/023411, the content of which is hereby incorporated by reference. Any alpha-amylase described or referenced herein is contemplated for expression in the host cell or fermenting organism.

In some embodiments, the host cell or fermenting organism comprising a heterologous polynucleotide encoding an alpha-amylase has an increased level of alpha-amylase activity compared to the host cells without the heterologous polynucleotide encoding the alpha-amylase, when cultivated under the same conditions. In some embodiments, the host cell or fermenting organism has an increased level of alpha-amylase activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the alpha-amylase, when cultivated under the same conditions (e.g., as described in Example 2).

Exemplary alpha-amylases that can be used with the host cells and/or the methods described herein include bacterial, yeast, or filamentous fungal alpha-amylases, e.g., derived from any of the microorganisms described or referenced herein.

The term “bacterial alpha-amylase” means any bacterial alpha-amylase classified under EC 3.2.1.1. A bacterial alpha-amylase used herein may, e.g., be derived from a strain of the genus Bacillus, which is sometimes also referred to as the genus Geobacillus. In one embodiment, the Bacillus alpha-amylase is derived from a strain of Bacillus amyloliquefaciens, Bacillus licheniformis, Bacillus stearothermophilus, or Bacillus subtilis, but may also be derived from other Bacillus sp.

Specific examples of bacterial alpha-amylases include the Bacillus stearothermophilus alpha-amylase (BSG) of SEQ ID NO: 3 in WO 99/19467, the Bacillus amyloliquefaciens alpha-amylase (BAN) of SEQ ID NO: 5 in WO 99/19467, and the Bacillus licheniformis alpha-amylase (BLA) of SEQ ID NO: 4 in WO 99/19467 (all sequences are hereby incorporated by reference). In one embodiment, the alpha-amylase may be an enzyme having a mature polypeptide sequence with a degree of identity of at least 60%, e.g., at least 70%, at least 80%, at least 90%, at least 95%, at least 96%, at least 97%, at least 98% or at least 99% to any of the sequences shown in SEQ ID NOs: 3, 4 or 5, in WO 99/19467.

In one embodiment, the alpha-amylase is derived from Bacillus stearothermophilus. The Bacillus stearothermophilus alpha-amylase may be a mature wild-type or a mature variant thereof. The mature Bacillus stearothermophilus alpha-amylases may naturally be truncated during recombinant production. For instance, the Bacillus stearothermophilus alpha-amylase may be a truncated at the C-terminal, so that it is from 480-495 amino acids long, such as about 491 amino acids long, e.g., so that it lacks a functional starch binding domain (compared to SEQ ID NO: 3 in WO 99/19467).

The Bacillus alpha-amylase may also be a variant and/or hybrid. Examples of such a variant can be found in any of WO 96/23873, WO 96/23874, WO 97/41213, WO 99/19467, WO 00/60059, and WO 02/10355 (each hereby incorporated by reference). Specific alpha-amylase variants are disclosed in U.S. Pat. Nos. 6,093,562, 6,187,576, 6,297,038, and 7,713,723 (hereby incorporated by reference) and include Bacillus stearothermophilus alpha-amylase (often referred to as BSG alpha-amylase) variants having a deletion of one or two amino acids at positions R179, G180, I181 and/or G182, preferably a double deletion disclosed in WO 96/23873—see, e.g., page 20, lines 1-10 (hereby incorporated by reference), such as corresponding to deletion of positions I181 and G182 compared to the amino acid sequence of Bacillus stearothermophilus alpha-amylase set forth in SEQ ID NO: 3 disclosed in WO 99/19467 or the deletion of amino acids R179 and G180 using SEQ ID NO: 3 in WO 99/19467 for numbering (which reference is hereby incorporated by reference). In some embodiments, the Bacillus alpha-amylases, such as Bacillus stearothermophilus alpha-amylases, have a double deletion corresponding to a deletion of positions 181 and 182 and further optionally comprise a N193F substitution (also denoted I181*+G182*+N193F) compared to the wild-type BSG alpha-amylase amino acid sequence set forth in SEQ ID NO: 3 disclosed in WO 99/19467. The bacterial alpha-amylase may also have a substitution in a position corresponding to S239 in the Bacillus licheniformis alpha-amylase shown in SEQ ID NO: 4 in WO 99/19467, or a S242 and/or E188P variant of the Bacillus stearothermophilus alpha-amylase of SEQ ID NO: 3 in WO 99/19467.

In one embodiment, the variant is a S242A, E or Q variant, e.g., a S242Q variant, of the Bacillus stearothermophilus alpha-amylase.

In one embodiment, the variant is a position E188 variant, e.g., E188P variant of the Bacillus stearothermophilus alpha-amylase.

The bacterial alpha-amylase may, in one embodiment, be a truncated Bacillus alpha-amylase. In one embodiment, the truncation is so that, e.g., the Bacillus stearothermophilus alpha-amylase shown in SEQ ID NO: 3 in WO 99/19467, is about 491 amino acids long, such as from 480 to 495 amino acids long, or so it lacks a functional starch bind domain.

The bacterial alpha-amylase may also be a hybrid bacterial alpha-amylase, e.g., an alpha-amylase comprising 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467). In one embodiment, this hybrid has one or more, especially all, of the following substitutions: G48A+T49I+G107A+H156Y+A181T+N190F+I201F+A209V+Q264S (using the Bacillus licheniformis numbering in SEQ ID NO: 4 of WO 99/19467). In some embodiments, the variants have one or more of the following mutations (or corresponding mutations in other Bacillus alpha-amylases): H154Y, A181T, N190F, A209V and Q264S and/or the deletion of two residues between positions 176 and 179, e.g., deletion of E178 and G179 (using SEQ ID NO: 5 of WO 99/19467 for position numbering).

In one embodiment, the bacterial alpha-amylase is the mature part of the chimeric alpha-amylase disclosed in Richardson et al. (2002), The Journal of Biological Chemistry, Vol. 277, No 29, Issue 19 July, pp. 267501-26507, referred to as BD5088 or a variant thereof. This alpha-amylase is the same as the one shown in SEQ ID NO: 2 in WO2007/134207. The mature enzyme sequence starts after the initial “Met” amino acid in position 1.

The alpha-amylase may be a thermostable alpha-amylase, such as a thermostable bacterial alpha-amylase, e.g., from Bacillus stearothermophilus. In one embodiment, the alpha-amylase used in a process described herein has a T½ (min) at pH 4.5, 85° C., 0.12 mM CaCl₂ of at least 10 determined as described in Example 1 of WO2018/098381.

In one embodiment, the thermostable alpha-amylase has a T½ (min) at pH 4.5, 85° C., 0.12 mM CaCl₂, of at least 15. In one embodiment, the thermostable alpha-amylase has a T½ (min) at pH 4.5, 85° C., 0.12 mM CaCl₂, of as at least 20. In one embodiment, the thermostable alpha-amylase has a T½ (min) at pH 4.5, 85° C., 0.12 mM CaCl₂, of as at least 25. In one embodiment, the thermostable alpha-amylase has a T½ (min) at pH 4.5, 85° C., 0.12 mM CaCl₂, of as at least 30. In one embodiment, the thermostable alpha-amylase has a T½ (min) at pH 4.5, 85° C., 0.12 mM CaCl₂, of as at least 40.

In one embodiment, the thermostable alpha-amylase has a T½ (min) at pH 4.5, 85° C., 0.12 mM CaCl₂, of at least 50. In one embodiment, the thermostable alpha-amylase has a T½ (min) at pH 4.5, 85° C., 0.12 mM CaCl₂, of at least 60. In one embodiment, the thermostable alpha-amylase has a T½ (min) at pH 4.5, 85° C., 0.12 mM CaCl₂, between 10-70. In one embodiment, the thermostable alpha-amylase has a T½ (min) at pH 4.5, 85° C., 0.12 mM CaCl₂, between 15-70. In one embodiment, the thermostable alpha-amylase has a T½ (min) at pH 4.5, 85° C., 0.12 mM CaCl₂, between 20-70. In one embodiment, the thermostable alpha-amylase has a T½ (min) at pH 4.5, 85° C., 0.12 mM CaCl₂, between 25-70. In one embodiment, the thermostable alpha-amylase has a T½ (min) at pH 4.5, 85° C., 0.12 mM CaCl₂, between 30-70. In one embodiment, the thermostable alpha-amylase has a T½ (min) at pH 4.5, 85° C., 0.12 mM CaCl₂, between 40-70. In one embodiment, the thermostable alpha-amylase has a T½ (min) at pH 4.5, 85° C., 0.12 mM CaCl₂, between 50-70. In one embodiment, the thermostable alpha-amylase has a T½ (min) at pH 4.5, 85° C., 0.12 mM CaCl₂, between 60-70.

In one embodiment, the alpha-amylase is a bacterial alpha-amylase, e.g., derived from the genus Bacillus, such as a strain of Bacillus stearothermophilus, e.g., the Bacillus stearothermophilus as disclosed in WO 99/019467 as SEQ ID NO: 3 with one or two amino acids deleted at positions R179, G180, I181 and/or G182, in particular with R179 and G180 deleted, or with I181 and G182 deleted, with mutations in below list of mutations.

In some embodiment, the Bacillus stearothermophilus alpha-amylases have double deletion I181+G182, and optional substitution N193F, further comprising one of the following substitutions or combinations of substitutions:

V59A+Q89R+G112D+E129V+K177L+R179E+K220P+N224L+Q254S;

V59A+Q89R+E129V+K177L+R179E+H208Y+K220P+N224L+Q254S;

V59A+Q89R+E129V+K177L+R179E+K220P+N224L+Q254S+D269E+D281N;

V59A+Q89R+E129V+K177L+R179E+K220P+N224L+Q254S+I270L;

V59A+Q89R+E129V+K177L+R179E+K220P+N224L+Q254S+H274K;

V59A+Q89R+E129V+K177L+R179E+K220P+N224L+Q254S+Y276F;

V59A+E129V+R157Y+K177L+R179E+K220P+N224L+S242Q+Q254S;

V59A+E129V+K177L+R179E+H208Y+K220P+N224L+S242Q+Q254S;

V59A+E129V+K177L+R179E+K220P+N224L+S242Q+Q254S;

V59A+E129V+K177L+R179E+K220P+N224L+S242Q+Q254S+H274K;

V59A+E129V+K177L+R179E+K220P+N224L+S242Q+Q254S+Y276F;

V59A+E129V+K177L+R179E+K220P+N224L+S242Q+Q254S+D281N;

V59A+E129V+K177L+R179E+K220P+N224L+S242Q+Q254S+M284T;

V59A+E129V+K177L+R179E+K220P+N224L+S242Q+Q254S+G416V;

V59A+E129V+K177L+R179E+K220P+N224L+Q254S;

V59A+E129V+K177L+R179E+K220P+N224L+Q254S+M284T;

A91L+M961+E129V+K177L+R179E+K220P+N224L+S242Q+Q254S;

E129V+K177L+R179E;

E129V+K177L+R179E+K220P+N224L+S242Q+Q254S;

E129V+K177L+R179E+K220P+N224L+S242Q+Q254S+Y276F+L427M;

E129V+K177L+R179E+K220P+N224L+S242Q+Q254S+M284T;

E129V+K177L+R179E+K220P+N224L+S242Q+Q254S+N376*+I377*;

E129V+K177L+R179E+K220P+N224L+Q254S;

E129V+K177L+R179E+K220P+N224L+Q254S+M284T;

E129V+K177L+R179E+S242Q;

E129V+K177L+R179V+K220P+N224L+S242Q+Q254S;

K220P+N224L+S242Q+Q254S;

M284V;

V59A+Q89R+E129V+K177L+R179E+Q254S+M284V; and

V59A+E129V+K177L+R179E+Q254S+M284V;

In one embodiment, the alpha-amylase is selected from the group of Bacillus stearothermophilus alpha-amylase variants with double deletion I181*+G182*, and optionally substitution N193F, and further one of the following substitutions or combinations of substitutions:

E129V+K177L+R179E;

V59A+Q89R+E129V+K177L+R179E+H208Y+K220P+N224L+Q254S;

V59A+Q89R+E129V+K177L+R179E+Q254S+M284V;

V59A+E129V+K177L+R179E+Q254S+M284V; and

E129V+K177L+R179E+K220P+N224L+S242Q+Q254S (using SEQ ID NO: 1 herein for numbering).

It should be understood that when referring to Bacillus stearothermophilus alpha-amylase and variants thereof they are normally produced in truncated form. In particular, the truncation may be so that the Bacillus stearothermophilus alpha-amylase shown in SEQ ID NO: 3 in WO 99/19467, or variants thereof, are truncated in the C-terminal and are typically from 480-495 amino acids long, such as about 491 amino acids long, e.g., so that it lacks a functional starch binding domain.

In one embodiment, the alpha-amylase variant may be an enzyme having a mature polypeptide sequence with a degree of identity of at least 60%, e.g., at least 70%, at least 80%, at least 90%, at least 95%, at least 91%, at least 92%, at least 93%, at least 94%, at least 95%, at least 96%, at least 97%, at least 98% or at least 99%, but less than 100% to the sequence shown in SEQ ID NO: 3 in WO 99/19467.

In one embodiment, the bacterial alpha-amylase, e.g., Bacillus alpha-amylase, such as especially Bacillus stearothermophilus alpha-amylase, or variant thereof, is dosed to liquefaction in a concentration between 0.01-10 KNU-A/g DS, e.g., between 0.02 and 5 KNU-A/g DS, such as 0.03 and 3 KNU-A, preferably 0.04 and 2 KNU-A/g DS, such as especially 0.01 and 2 KNU-A/g DS. In one embodiment, the bacterial alpha-amylase, e.g., Bacillus alpha-amylase, such as especially Bacillus stearothermophilus alpha-amylases, or variant thereof, is dosed to liquefaction in a concentration of between 0.0001-1 mg EP (Enzyme Protein)/g DS, e.g., 0.0005-0.5 mg EP/g DS, such as 0.001-0.1 mg EP/g DS.

In one embodiment, the bacterial alpha-amylase is derived from the Bacillus subtilis alpha-amylase of SEQ ID NO: 76, the Bacillus subtilis alpha-amylase of SEQ ID NO: 82, the Bacillus subtilis alpha-amylase of SEQ ID NO: 83, the Bacillus subtilis alpha-amylase of SEQ ID NO: 84, or the Bacillus licheniformis alpha-amylase of SEQ ID NO: 85, the Clostridium phytofermentans alpha-amylase of SEQ ID NO: 89, the Clostridium phytofermentans alpha-amylase of SEQ ID NO: 90, the Clostridium phytofermentans alpha-amylase of SEQ ID NO: 91, the Clostridium phytofermentans alpha-amylase of SEQ ID NO: 92, the Clostridium phytofermentans alpha-amylase of SEQ ID NO: 93, the Clostridium phytofermentans alpha-amylase of SEQ ID NO: 94, the Clostridium thermocellum alpha-amylase of SEQ ID NO: 95, the Thermobifida fusca alpha-amylase of SEQ ID NO: 96, the Thermobifida fusca alpha-amylase of SEQ ID NO: 97, the Anaerocellum thermophilum of SEQ ID NO: 98, the Anaerocellum thermophilum of SEQ ID NO: 99, the Anaerocellum thermophilum of SEQ ID NO: 100, the Streptomyces avermitilis of SEQ ID NO: 101, or the Streptomyces avermitilis of SEQ ID NO: 88.

In one embodiment, the alpha-amylase is derived from Bacillus amyloliquefaciens, such as the Bacillus amyloliquefaciens alpha-amylase of SEQ ID NO: 231 (e.g., as described in WO2018/002360, or variants thereof as described in WO2017/037614).

In one embodiment, the alpha-amylase is derived from a yeast alpha-amylase, such as the Saccharomycopsis fibuligera alpha-amylase of SEQ ID NO: 77, the Debaryomyces occidentalis alpha-amylase of SEQ ID NO: 78, the Debaryomyces occidentalis alpha-amylase of SEQ ID NO: 79, the Lipomyces kononenkoae alpha-amylase of SEQ ID NO: 80, the Lipomyces kononenkoae alpha-amylase of SEQ ID NO: 81.

In one embodiment, the alpha-amylase is derived from a filamentous fungal alpha-amylase, such as the Aspergillus niger alpha-amylase of SEQ ID NO: 86, or the Aspergillus niger alpha-amylase of SEQ ID NO: 87.

Additional alpha-amylases that may be expressed with the host cells and fermenting organisms and used with the methods described herein are described in the examples, and include, but are not limited to alpha-amylases shown in Table 4 (or derivatives thereof).

TABLE 4

| Donor Organism | SEQ ID NO: |
|---|---|
| (catalytic domain) | (mature polypeptide) |
| Rhizomucor pusillus | 121 |
| Bacillus licheniformis | 122 |
| Aspergillus niger | 123 |
| Aspergillus tamarii | 124 |
| Acidomyces richmondensis | 125 |
| Aspergillus bombycis | 126 |
| Alternaria sp | 127 |
| Rhizopus microsporus | 128 |
| Syncephalastrum racemosum | 129 |
| Rhizomucor pusillus | 130 |
| Dichotomocladium hesseltinei | 131 |
| Lichtheimia ramosa | 132 |
| Penicillium aethiopicum | 133 |
| Subulispora sp | 134 |
| Trichoderma paraviridescens | 135 |
| Byssoascus striatosporus | 136 |
| Aspergillus brasiliensis | 137 |
| Penicillium subspinulosum | 138 |
| Penicillium antarcticum | 139 |
| Penicillium coprophilum | 140 |
| Penicillium olsonii | 141 |
| Penicillium vasconiae | 142 |
| Penicillium sp | 143 |
| Heterocephalum aurantiacum | 144 |
| Neosartorya massa | 145 |
| Penicillium janthinellum | 146 |
| Aspergillus brasiliensis | 147 |
| Aspergillus westerdijkiae | 148 |
| Hamigera avellanea | 149 |
| Hamigera avellanea | 150 |
| Meripilus giganteus | 151 |
| Cerrena unicolor | 152 |
| Physalacria cryptomeriae | 153 |
| Lenzites betulinus | 154 |
| Trametes ljubarskyi | 155 |
| Bacillus subtilis | 156 |
| Bacillus subtilis subsp. subtilis | 157 |
| Schwanniomyces occidentalis | 158 |
| Rhizomucor pusillus | 159 |
| Aspergillus niger | 160 |
| Bacillus stearothermophilus | 161 |
| Bacillus halmapalus | 162 |
| Aspergillus oryzae | 163 |
| Bacillus amyloliquefaciens | 164 |
| Rhizomucor pusillus | 165 |
| Kionochaeta ivoriensis | 166 |
| Aspergillus niger | 167 |
| Aspergillus oryzae | 168 |
| Penicillium canescens | 169 |
| Acidomyces acidothermus | 170 |
| Kinochaeta ivoriensis | 171 |
| Aspergillus terreus | 172 |
| Thamnidium elegans | 173 |
| Meripilus giganteus | 174 |
| Bacillus amyloliquefaciens | 231 |
| Thermococcus gammatolerans | 251 |
| Thermococcus thioreducens | 252 |
| Thermococcus eurythermalis | 253 |
| Thermococcus hydrothermalis | 254 |
| Pyrococcus furiosus | 255 |
| Bacillus amyloliquefaciens | 256 |

Additional alpha-amylases contemplated for use with the present invention can be found in WO2011/153516, WO2017/087330 and WO2020/023411 (the content of which is incorporated herein).

Additional polynucleotides encoding suitable alpha-amylases may be obtained from microorganisms of any genus, including those readily available within the UniProtKB database.

The alpha-amylase coding sequences can also be used to design nucleic acid probes to identify and clone DNA encoding trehalases from strains of different genera or species, as described supra.

The polynucleotides encoding alpha-amylases may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding alpha-amylases are described supra.

In one embodiment, the alpha-amylase has a mature polypeptide sequence that comprises or consists of the amino acid sequence of any one of the alpha-amylases described or referenced herein (e.g., any one of SEQ ID NOs: 76-101, 121-174, 231 and 251-256). In another embodiment, the alpha-amylase has a mature polypeptide sequence that is a fragment of the any one of the alpha-amylases described or referenced herein (e.g., any one of SEQ ID NOs: 76-101,121-174, 231 and 251-256). In one embodiment, the number of amino acid residues in the fragment is at least 75%, e.g., at least 80%, 85%, 90%, or 95% of the number of amino acid residues in referenced full length alpha-amylase (e.g. any one of SEQ ID NOs: 76-101, 121-174, 231 and 251-256). In other embodiments, the alpha-amylase may comprise the catalytic domain of any alpha-amylase described or referenced herein (e.g., the catalytic domain of any one of SEQ ID NOs: 76-101, 121-174, 231 and 251-256).

The alpha-amylase may be a variant of any one of the alpha-amylases described supra (e.g., any one of SEQ ID NOs: 76-101, 121-174, 231 and 251-256). In one embodiment, the alpha-amylase has a mature polypeptide sequence of at least 60%, e.g., at least 65%, 70%, 75%, 80%, 85%, 90%, 95%, 97%, 98%, 99%, or 100% sequence identity to any one of the alpha-amylases described supra (e.g., any one of SEQ ID NOs: 76-101, 121-174, 231 and 251-256).

Examples of suitable amino acid changes, such as conservative substitutions that do not significantly affect the folding and/or activity of the alpha-amylase, are described herein.

In one embodiment, the alpha-amylase has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the alpha-amylases described supra (e.g., any one of SEQ ID NOs: 76-101,121-174,231 and 251-256). In one embodiment, the alpha-amylase has an amino acid substitution, deletion, and/or insertion of one or more (e.g., two, several) of amino acid sequence of any one of the alpha-amylases described supra (e.g., any one of SEQ ID NOs: 76-101, 121-174, 231 and 251-256). In some embodiments, the total number of amino acid substitutions, deletions and/or insertions is not more than 10, e.g., not more than 9, 8, 7, 6, 5, 4, 3, 2, or 1.

In some embodiments, the alpha-amylase has at least 20%, e.g., at least 40%, at least 50%, at least 60%, at least 70%, at least 80%, at least 90%, at least 95%, at least 96%, at least 97%, at least 98%, at least 99%, or 100% of the alpha-amylase activity of any alpha-amylase described or referenced herein (e.g., any one of SEQ ID NOs: 76-101, 121-174, 231 and 251-256) under the same conditions.

In one embodiment, the alpha-amylase coding sequence hybridizes under at least low stringency conditions, e.g., medium stringency conditions, medium-high stringency conditions, high stringency conditions, or very high stringency conditions with the full-length complementary strand of the coding sequence from any alpha-amylase described or referenced herein (e.g., any one of SEQ ID NOs: 76-101, 121-174 and 231). In one embodiment, the alpha-amylase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any alpha-amylase described or referenced herein (e.g., any one of SEQ ID NOs: 76-101, 121-174, 231 and 251-256).

In one embodiment, the alpha-amylase comprises the coding sequence of any alpha-amylase described or referenced herein (any one of SEQ ID NOs: 76-101, 121-174, 231 and 251-256). In one embodiment, the alpha-amylase comprises a coding sequence that is a subsequence of the coding sequence from any alpha-amylase described or referenced herein, wherein the subsequence encodes a polypeptide having alpha-amylase activity. In one embodiment, the number of nucleotides residues in the subsequence is at least 75%, e.g., at least 80%, 85%, 90%, or 95% of the number of the referenced coding sequence.

The referenced alpha-amylase coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The alpha-amylase can also include fused polypeptides or cleavable fusion polypeptides, as described supra. In some embodiments, the alpha-amylase is a fusion protein comprising a signal peptide linked to the N-terminus of a mature polypeptide, such as any signal sequences described in U.S. Provisional Application No. 62/883,519 filed Aug. 6, 2019 and entitled “Fusion Proteins For Improved Enzyme Expression” (the content of which is hereby incorporated by reference).

### Phospholipases

The host cells and fermenting organisms may express a heterologous phospholipase. The phospholipase may be any phospholipase that is suitable for the host cells, fermenting organism, and/or the methods described herein, such as a naturally occurring phospholipase (e.g., a native phospholipase from another species or an endogenous phospholipase expressed from a modified expression vector) or a variant thereof that retains phospholipase activity. Any phospholipase contemplated for expression by a host cell or fermenting organism described below is also contemplated for embodiments of the invention involving exogenous addition of a phospholipase (e.g., added before, during or after liquefaction and/or saccharification).

In some embodiments, the host cell or fermenting organism comprises a heterologous polynucleotide encoding a phospholipase, for example, as described in WO2018/075430, the content of which is hereby incorporated by reference. In some embodiments, the phospholipase is classified as a phospholipase A. In other embodiments, the phospholipase is classified as a phospholipase C. Any phospholipase described or referenced herein is contemplated for expression in the host cell or fermenting organism.

In some embodiments, the host cell or fermenting organism comprising a heterologous polynucleotide encoding a phospholipase has an increased level of phospholipase activity compared to the host cells without the heterologous polynucleotide encoding the phospholipase, when cultivated under the same conditions. In some embodiments, the host cell or fermenting organism has an increased level of phospholipase activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the phospholipase, when cultivated under the same conditions.

Exemplary phospholipases that can be used with the host cells and/or the methods described herein include bacterial, yeast, or filamentous fungal phospholipases, e.g., derived from any of the microorganisms described or referenced herein.

Additional phospholipases that may be expressed with the host cells and fermenting organisms, and used with the methods described herein, and include, but are not limited to phospholipases shown in Table 5 (or derivatives thereof).

TABLE 5

| Donor Organism | SEQ ID NO: |
|---|---|
| (catalytic domain) | (mature polypeptide) |
| Thermomyces lanuginosus | 235 |
| Talaromyces leycettanus | 236 |
| Penicillium emersonii | 237 |
| Bacillus thuringiensis | 238 |
| Pseudomonas sp. | 239 |
| Kionochaeta sp. | 240 |
| Mariannaea pinicola | 241 |
| Fictibacillus macauensis | 242 |

Additional phospholipases contemplated for use with the present invention can be found in WO02018/075430 (the content of which is incorporated herein).

Additional polynucleotides encoding suitable phospholipases may be obtained from microorganisms of any genus, including those readily available within the UniProtKB database.

The phospholipase coding sequences can also be used to design nucleic acid probes to identify and clone DNA encoding phospholipases from strains of different genera or species, as described supra.

The polynucleotides encoding phospholipases may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding phospholipases are described supra.

In one embodiment, the phospholipase has a mature polypeptide sequence that comprises or consists of the amino acid sequence of any one of the phospholipases described or referenced herein (e.g., any one of SEQ ID NOs: 235, 236, 237, 238, 239, 240, 241, and 242). In another embodiment, the phospholipase has a mature polypeptide sequence that is a fragment of the any one of the phospholipases described or referenced herein (e.g., any one of SEQ ID NOs: 235, 236, 237, 238, 239, 240, 241, and 242). In one embodiment, the number of amino acid residues in the fragment is at least 75%, e.g., at least 80%, 85%, 90%, or 95% of the number of amino acid residues in referenced full length phospholipase (e.g. any one of SEQ ID NOs: 235, 236, 237, 238, 239, 240, 241, and 242). In other embodiments, the phospholipase may comprise the catalytic domain of any phospholipase described or referenced herein (e.g., the catalytic domain of any one of SEQ ID NOs: 235, 236, 237, 238, 239, 240, 241, and 242).

The phospholipase may be a variant of any one of the phospholipases described supra (e.g., any one of SEQ ID NOs: SEQ ID NOs: 235, 236, 237, 238, 239, 240, 241, and 242). In one embodiment, the phospholipase has a mature polypeptide sequence of at least 60%, e.g., at least 65%, 70%, 75%, 80%, 85%, 90%, 95%, 97%, 98%, 99%, or 100% sequence identity to any one of the phospholipases described supra (e.g., any one of SEQ ID NOs: 235, 236, 237, 238, 239, 240, 241, and 242).

Examples of suitable amino acid changes, such as conservative substitutions that do not significantly affect the folding and/or activity of the phospholipase, are described herein.

In one embodiment, the phospholipase has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the phospholipases described supra (e.g., any one of SEQ ID NOs: 235, 236, 237, 238, 239, 240, 241, and 242). In one embodiment, the phospholipase has an amino acid substitution, deletion, and/or insertion of one or more (e.g., two, several) of amino acid sequence of any one of the phospholipases described supra (e.g., any one of SEQ ID NOs: 235, 236, 237, 238, 239, 240, 241, and 242). In some embodiments, the total number of amino acid substitutions, deletions and/or insertions is not more than 10, e.g., not more than 9, 8, 7, 6, 5, 4, 3, 2, or 1.

In some embodiments, the phospholipase has at least 20%, e.g., at least 40%, at least 50%, at least 60%, at least 70%, at least 80%, at least 90%, at least 95%, at least 96%, at least 97%, at least 98%, at least 99%, or 100% of the phospholipase activity of any phospholipase described or referenced herein (e.g., any one of SEQ ID NOs: 235, 236, 237, 238, 239, 240, 241, and 242) under the same conditions.

In one embodiment, the phospholipase coding sequence hybridizes under at least low stringency conditions, e.g., medium stringency conditions, medium-high stringency conditions, high stringency conditions, or very high stringency conditions with the full-length complementary strand of the coding sequence from any phospholipase described or referenced herein (e.g., a coding sequence for a phospholipase of SEQ ID NO: 235, 236, 237, 238, 239, 240, 241 or 242). In one embodiment, the phospholipase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any phospholipase described or referenced herein (e.g., a coding sequence for a phospholipase of SEQ ID NO: 235, 236, 237, 238, 239, 240, 241 or 242).

In one embodiment, the phospholipase comprises the coding sequence of any phospholipase described or referenced herein (e.g., a coding sequence for a phospholipase of SEQ ID NO: 235, 236, 237, 238, 239, 240, 241 or 242). In one embodiment, the phospholipase comprises a coding sequence that is a subsequence of the coding sequence from any phospholipase described or referenced herein, wherein the subsequence encodes a polypeptide having phospholipase activity. In one embodiment, the number of nucleotides residues in the subsequence is at least 75%, e.g., at least 80%, 85%, 90%, or 95% of the number of the referenced coding sequence.

The referenced phospholipase coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The phospholipase can also include fused polypeptides or cleavable fusion polypeptides, as described supra. In some embodiments, the phospholipase is a fusion protein comprising a signal peptide linked to the N-terminus of a mature polypeptide, such as any signal sequences described in U.S. Provisional Application No. 62/883,519 filed Aug. 6, 2019 and entitled “Fusion Proteins For Improved Enzyme Expression” (the content of which is hereby incorporated by reference).

### Trehalases

The host cells and fermenting organisms may express a heterologous trehalase. The trehalase can be any trehalase that is suitable for the host cells, fermenting organisms and/or their methods of use described herein, such as a naturally occurring trehalase or a variant thereof that retains trehalase activity. Any trehalase contemplated for expression by a host cell or fermenting organism described below is also contemplated for embodiments of the invention involving exogenous addition of a trehalase (e.g., added before, during or after liquefaction and/or saccharification).

In some embodiments, the host cell or fermenting organism comprising a heterologous polynucleotide encoding a trehalase has an increased level of trehalase activity compared to the host cells without the heterologous polynucleotide encoding the trehalase, when cultivated under the same conditions. In some embodiments, the host cell or fermenting organism has an increased level of trehalase activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the trehalase, when cultivated under the same conditions.

Trehalases that may be expressed with the host cells and fermenting organisms, and used with the methods described herein include, but are not limited to, trehalases shown in Table 6 (or derivatives thereof).

TABLE 6

| Donor Organism | SEQ ID NO: |
|---|---|
| (catalytic domain) | (mature polypeptide) |
| Chaetomium megalocarpum | 175 |
| Lecanicillium psalliotae | 176 |
| Doratomyces sp | 177 |
| Mucor moelleri | 178 |
| Phialophora cyclaminis | 179 |
| Thielavia arenaria | 180 |
| Thielavia antarctica | 181 |
| Chaetomium sp | 182 |
| Chaetomium nigricolor | 183 |
| Chaetomium jodhpurense | 184 |
| Chaetomium piluliferum | 185 |
| Myceliophthora hinnulea | 186 |
| Chloridium virescens | 187 |
| Gelasinospora cratophora | 188 |
| Acidobacteriaceae bacterium | 189 |
| Acidobacterium capsulatum | 190 |
| Acidovorax wautersii | 191 |
| Xanthomonas arboricola | 192 |
| Kosakonia sacchari | 193 |
| Enterobacter sp | 194 |
| Saitozyma flava | 195 |
| Phaeotremella skinneri | 196 |
| Trichoderma asperellum | 197 |
| Corynascus sepedonium | 198 |
| Myceliophthora thermophila | 199 |
| Trichoderma reesei | 200 |
| Chaetomium virescens | 201 |
| Rhodothermus marinus | 202 |
| Myceliophthora sepedonium | 203 |
| Moelleriella libera | 204 |
| Acremonium dichromosporum | 205 |
| Fusarium sambucinum | 206 |
| Phoma sp | 207 |
| Lentinus similis | 208 |
| Diaporthe nobilis | 209 |
| Solicoccozyma terricola | 210 |
| Dioszegia cryoxerica | 211 |
| Talaromyces funiculosus | 212 |
| Hamigera avellanea | 213 |
| Talaromyces ruber | 214 |
| Trichoderma lixii | 215 |
| Aspergillus cervinus | 216 |
| Rasamsonia brevistipitata | 217 |
| Acremonium curvulum | 218 |
| Talaromyces piceae | 219 |
| Penicillium sp | 220 |
| Talaromyces aurantiacus | 221 |
| Talaromyces pinophilus | 222 |
| Talaromyces leycettanus | 223 |
| Talaromyces variabilis | 224 |
| Aspergillus niger | 225 |
| Trichoderma reesei | 226 |

Additional polynucleotides encoding suitable trehalases may be derived from microorganisms of any suitable genus, including those readily available within the UniProtKB database.

The trehalase coding sequences can also be used to design nucleic acid probes to identify and clone DNA encoding trehalases from strains of different genera or species, as described supra.

The polynucleotides encoding trehalases may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding trehalases are described supra.

In one embodiment, the trehalase has a mature polypeptide sequence that comprises or consists of the amino acid sequence of any one of the trehalases described or referenced herein (e.g., any one of SEQ ID NOs: 175-226). In another embodiment, the trehalase has a mature polypeptide sequence that is a fragment of the any one of the trehalases described or referenced herein (e.g., any one of SEQ ID NOs: 175-226). In one embodiment, the number of amino acid residues in the fragment is at least 75%, e.g., at least 80%, 85%, 90%, or 95% of the number of amino acid residues in referenced full length trehalase (e.g. any one of SEQ ID NOs: 175-226). In other embodiments, the trehalase may comprise the catalytic domain of any trehalase described or referenced herein (e.g., the catalytic domain of any one of SEQ ID NOs: 175-226).

The trehalase may be a variant of any one of the trehalases described supra (e.g., any one of SEQ ID NOs: 175-226). In one embodiment, the trehalase has a mature polypeptide sequence of at least 60%, e.g., at least 65%, 70%, 75%, 80%, 85%, 90%, 95%, 97%, 98%, 99%, or 100% sequence identity to any one of the trehalases described supra (e.g., any one of SEQ ID NOs: 175-226).

Examples of suitable amino acid changes, such as conservative substitutions that do not significantly affect the folding and/or activity of the trehalase, are described herein.

In one embodiment, the trehalase has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the trehalases described supra (e.g., any one of SEQ ID NOs: 175-226). In one embodiment, the trehalase has an amino acid substitution, deletion, and/or insertion of one or more (e.g., two, several) of amino acid sequence of any one of the trehalases described supra (e.g., any one of SEQ ID NOs: 175-226). In some embodiments, the total number of amino acid substitutions, deletions and/or insertions is not more than 10, e.g., not more than 9, 8, 7, 6, 5, 4, 3, 2, or 1.

In some embodiments, the trehalase has at least 20%, e.g., at least 40%, at least 50%, at least 60%, at least 70%, at least 80%, at least 90%, at least 95%, at least 96%, at least 97%, at least 98%, at least 99%, or 100% of the trehalase activity of any trehalase described or referenced herein (e.g., any one of SEQ ID NOs: 175-226) under the same conditions.

In one embodiment, the trehalase coding sequence hybridizes under at least low stringency conditions, e.g., medium stringency conditions, medium-high stringency conditions, high stringency conditions, or very high stringency conditions with the full-length complementary strand of the coding sequence from any trehalase described or referenced herein (e.g., any one of SEQ ID NOs: 175-226). In one embodiment, the trehalase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any trehalase described or referenced herein (e.g., any one of SEQ ID NOs: 175-226).

In one embodiment, the trehalase comprises the coding sequence of any trehalase described or referenced herein (any one of SEQ ID NOs: 175-226). In one embodiment, the trehalase comprises a coding sequence that is a subsequence of the coding sequence from any trehalase described or referenced herein, wherein the subsequence encodes a polypeptide having trehalase activity. In one embodiment, the number of nucleotides residues in the subsequence is at least 75%, e.g., at least 80%, 85%, 90%, or 95% of the number of the referenced coding sequence.

The referenced trehalase coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The trehalase can also include fused polypeptides or cleavable fusion polypeptides, as described supra. In some embodiments, the trehalase is a fusion protein comprising a signal peptide linked to the N-terminus of a mature polypeptide, such as any signal sequences described in U.S. Provisional Application No. 62/883,519 filed Aug. 6, 2019 and entitled “Fusion Proteins For Improved Enzyme Expression” (the content of which is hereby incorporated by reference).

### Proteases

The host cells and fermenting organisms may express a heterologous protease. The protease can be any protease that is suitable for the host cells and fermenting organisms and/or their methods of use described herein, such as a naturally occurring protease or a variant thereof that retains protease activity. Any protease contemplated for expression by a host cell or fermenting organism described below is also contemplated for embodiments of the invention involving exogenous addition of a protease (e.g., added before, during or after liquefaction and/or saccharification).

Proteases are classified on the basis of their catalytic mechanism into the following groups: Serine proteases (S), Cysteine proteases (C), Aspartic proteases (A), Metallo proteases (M), and Unknown, or as yet unclassified, proteases (U), see Handbook of Proteolytic Enzymes, A. J. Barrett, N. D. Rawlings, J. F. Woessner (eds), Academic Press (1998), in particular the general introduction part.

Protease activity can be measured using any suitable assay, in which a substrate is employed, that includes peptide bonds relevant for the specificity of the protease in question.

Assay-pH and assay-temperature are likewise to be adapted to the protease in question. Examples of assay-pH-values are pH 6, 7, 8, 9, 10, or 11. Examples of assay-temperatures are 30, 35, 37, 40, 45, 50, 55, 60, 65, 70 or 80° C.

In some embodiments, the host cell or fermenting organism comprising a heterologous polynucleotide encoding a protease has an increased level of protease activity compared to the host cell or fermenting organism without the heterologous polynucleotide encoding the protease, when cultivated under the same conditions. In some embodiments, the host cell or fermenting organism has an increased level of protease activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the protease, when cultivated under the same conditions.

Exemplary proteases that may be expressed with the host cells and fermenting organisms, and used with the methods described herein include, but are not limited to, proteases shown in Table 7 (or derivatives thereof).

TABLE 7

| Donor Organism | SEQ ID NO: | |
|---|---|---|
| (catalytic domain) | (mature polypeptide) | Family |
| Aspergillus niger | 9 | A1 |
| Trichoderma reesei | 10 | |
| Thermoascus aurantiacus | 11 | M35 |
| Dichomitus squalens | 12 | S53 |
| Nocardiopsis prasina | 13 | S1 |
| Penicillium simplicissimum | 14 | S10 |
| Aspergillus niger | 15 | |
| Meriphilus giganteus | 16 | S53 |
| Lecanicillium sp. WMM742 | 17 | S53 |
| Talaromyces proteolyticus | 18 | S53 |
| Penicillium ranomafanaense | 19 | A1A |
| Aspergillus oryzae | 20 | S53 |
| Talaromyces liani | 21 | S10 |
| Thermoascus thermophilus | 22 | S53 |
| Pyrococcus furiosus | 23 | |
| Trichoderma reesei | 24 | |
| Rhizomucor miehei | 25 | |
| Lenzites betulinus | 26 | S53 |
| Neolentinus lepideus | 27 | S53 |
| Thermococcus sp. | 28 | S8 |
| Thermococcus sp. | 29 | S8 |
| Thermomyces lanuginosus | 30 | S53 |
| Thermococcus thioreducens | 31 | S53 |
| Polyporus arcularius | 32 | S53 |
| Ganoderma lucidum | 33 | S53 |
| Ganoderma lucidum | 34 | S53 |
| Ganoderma lucidum | 35 | S53 |
| Trametes sp. AH28-2 | 36 | S53 |
| Cinereomyces lindbladii | 37 | S53 |
| Trametes versicolor O82DDP | 38 | S53 |
| Paecilomyces hepiali | 39 | S53 |
| Isaria tenuipes | 40 | S53 |
| Aspergillus tamarii | 41 | S53 |
| Aspergillus brasiliensis | 42 | S53 |
| Aspergillus iizukae | 43 | S53 |
| Penicillium sp-72364 | 44 | S10 |
| Aspergillus denticulatus | 45 | S10 |
| Hamigera sp. t184-6 | 46 | S10 |
| Penicillium janthinellum | 47 | S10 |
| Penicillium vasconiae | 48 | S10 |
| Hamigera paravellanea | 49 | S10 |
| Talaromyces variabilis | 50 | S10 |
| Penicillium arenicola | 51 | S10 |
| Nocardiopsis kunsanensis | 52 | S1 |
| Streptomyces parvulus | 53 | S1 |
| Saccharopolyspora endophytica | 54 | S1 |
| luteus cell wall enrichments K | 55 | S1 |
| Saccharothrix australiensis | 56 | S1 |
| Nocardiopsis baichengensis | 57 | S1 |
| Streptomyces sp. SM15 | 58 | S1 |
| Actinoalloteichus spitiensis | 59 | S1 |
| Byssochlamys verrucosa | 60 | M35 |
| Hamigera terricola | 61 | M35 |
| Aspergillus tamarii | 62 | M35 |
| Aspergillus niveus | 63 | M35 |
| Penicillium sclerotiorum | 64 | A1 |
| Penicillium bilaiae | 65 | A1 |
| Penicillium antarcticum | 66 | A1 |
| Penicillium sumatrense | 67 | A1 |
| Trichoderma lixii | 68 | A1 |
| Trichoderma brevicompactum | 69 | A1 |
| Penicillium cinnamopurpureum | 70 | A1 |
| Bacillus licheniformis | 71 | S8 |
| Bacillus subtilis | 72 | S8 |
| Trametes cf versicol | 73 | S53 |

Additional polynucleotides encoding suitable proteases may be derived from microorganisms of any suitable genus, including those readily available within the UniProtKB database.

In one embodiment, the protease is derived from Aspergillus, such as the Aspergillus niger protease of SEQ ID NO: 9, the Aspergillus tamarii protease of SEQ ID NO: 41, or the Aspergillus denticulatus protease of SEQ ID NO: 45. In one embodiment, the protease is derived from Dichomitus, such as the Dichomitus squalens protease of SEQ ID NO: 12. In one embodiment, the protease is derived from Penicillium, such as the Penicillium simplicissimum protease of SEQ ID NO: 14, the Penicillium antarcticum protease of SEQ ID NO: 66, or the Penicillium sumatrense protease of SEQ ID NO: 67. In one embodiment, the protease is derived from Meriphilus, such as the Meriphilus giganteus protease of SEQ ID NO: 16. In one embodiment, the protease is derived from Talaromyces, such as the Talaromyces liani protease of SEQ ID NO: 21. In one embodiment, the protease is derived from Thermoascus, such as the Thermoascus thermophilus protease of SEQ ID NO: 22. In one embodiment, the protease is derived from Ganoderma, such as the Ganoderma lucidum protease of SEQ ID NO: 33. In one embodiment, the protease is derived from Hamigera, such as the Hamigera terricola protease of SEQ ID NO: 61. In one embodiment, the protease is derived from Trichoderma, such as the Trichoderma brevicompactum protease of SEQ ID NO: 69.

The protease coding sequences can also be used to design nucleic acid probes to identify and clone DNA encoding proteases from strains of different genera or species, as described supra.

The polynucleotides encoding proteases may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding proteases are described supra.

In one embodiment, the protease has a mature polypeptide sequence that comprises or consists of the amino acid sequence of any one of SEQ ID NOs: 9-73 (e.g., any one of SEQ ID NOs: 9, 14, 16, 21, 22, 33, 41, 45, 61, 62, 66, 67, and 69; such as any one of SEQ NOs: 9, 14, 16, and 69). In another embodiment, the protease has a mature polypeptide sequence that is a fragment of the protease of any one of SEQ ID NOs: 9-73 (e.g., wherein the fragment has protease activity). In one embodiment, the number of amino acid residues in the fragment is at least 75%, e.g., at least 80%, 85%, 90%, or 95% of the number of amino acid residues in referenced full length protease (e.g. any one of SEQ ID NOs: 9-73). In other embodiments, the protease may comprise the catalytic domain of any protease described or referenced herein (e.g., the catalytic domain of any one of SEQ ID NOs: 9-73).

The protease may be a variant of any one of the proteases described supra (e.g., any one of SEQ ID NOs: 9-73. In one embodiment, the protease has a mature polypeptide sequence of at least 60%, e.g., at least 65%, 70%, 75%, 80%, 85%, 90%, 95%, 97%, 98%, 99%, or 100% sequence identity to any one of the proteases described supra (e.g., any one of SEQ ID NOs: 9-73).

Examples of suitable amino acid changes, such as conservative substitutions that do not significantly affect the folding and/or activity of the protease, are described herein.

In one embodiment, the protease has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the proteases described supra (e.g., any one of SEQ ID NOs: 9-73). In one embodiment, the protease has an amino acid substitution, deletion, and/or insertion of one or more (e.g., two, several) of amino acid sequence of any one of the proteases described supra (e.g., any one of SEQ ID NOs: 9-73). In some embodiments, the total number of amino acid substitutions, deletions and/or insertions is not more than 10, e.g., not more than 9, 8, 7, 6, 5, 4, 3, 2, or 1.

In one embodiment, the protease coding sequence hybridizes under at least low stringency conditions, e.g., medium stringency conditions, medium-high stringency conditions, high stringency conditions, or very high stringency conditions with the full-length complementary strand of the coding sequence from any protease described or referenced herein (e.g., any one of SEQ ID NOs: 9-73). In one embodiment, the protease coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any protease described or referenced herein (e.g., any one of SEQ ID NOs: 9-73).

In one embodiment, the protease comprises the coding sequence of any protease described or referenced herein (any one of SEQ ID NOs: 9-73). In one embodiment, the protease comprises a coding sequence that is a subsequence of the coding sequence from any protease described or referenced herein, wherein the subsequence encodes a polypeptide having protease activity. In one embodiment, the number of nucleotides residues in the subsequence is at least 75%, e.g., at least 80%, 85%, 90%, or 95% of the number of the referenced coding sequence.

The referenced protease coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The protease can also include fused polypeptides or cleavable fusion polypeptides, as described supra. In some embodiments, the protease is a fusion protein comprising a signal peptide linked to the N-terminus of a mature polypeptide, such as any signal sequences described in U.S. Provisional Application No. 62/883,519 filed Aug. 6, 2019 and entitled “Fusion Proteins For Improved Enzyme Expression” (the content of which is hereby incorporated by reference).

In one embodiment, the protease used according to a process described herein is a Serine proteases. In one particular embodiment, the protease is a serine protease belonging to the family 53, e.g., an endo-protease, such as S53 protease from Meiphilus giganteus, Dichomitus squalens Trametes versicolor, Polyporus arcularius, Lenzites betulinus, Ganoderma lucidum, Neolentinus lepideus, or Bacillus sp. 19138, in a process for producing ethanol from a starch-containing material, the ethanol yield was improved, when the S53 protease was present/or added during saccharification and/or fermentation of either gelatinized or un-gelatinized starch. In one embodiment, the proteases is selected from: (a) proteases belonging to the EC 3.4.21 enzyme group; and/or (b) proteases belonging to the EC 3.4.14 enzyme group; and/or (c) Serine proteases of the peptidase family S53 that comprises two different types of peptidases: tripeptidyl aminopeptidases (exo-type) and endo-peptidases; as described in 1993, Biochem. J. 290:205-218 and in MEROPS protease database, release, 9.4 (31 Jan. 2011) (www.merops.ac.uk). The database is described in Rawlings, N. D., Barrett, A. J. and Bateman, A., 2010, “MEROPS: the peptidase database”, Nucl. Acids Res. 38: D227-D233.

For determining whether a given protease is a Serine protease, and a family S53 protease, reference is made to the above Handbook and the principles indicated therein. Such determination can be carried out for all types of proteases, be it naturally occurring or wild-type proteases; or genetically engineered or synthetic proteases.

Peptidase family S53 contains acid-acting endopeptidases and tripeptidyl-peptidases. The residues of the catalytic triad are Glu, Asp, Ser, and there is an additional acidic residue, Asp, in the oxyanion hole. The order of the residues is Glu, Asp, Asp, Ser. The Ser residue is the nucleophile equivalent to Ser in the Asp, His, Ser triad of subtilisin, and the Glu of the triad is a substitute for the general base, His, in subtilisin.

The peptidases of the S53 family tend to be most active at acidic pH (unlike the homologous subtilisins), and this can be attributed to the functional importance of carboxylic residues, notably Asp in the oxyanion hole. The amino acid sequences are not closely similar to those in family S8 (i.e. serine endopeptidase subtilisins and homologues), and this, taken together with the quite different active site residues and the resulting lower pH for maximal activity, provides for a substantial difference to that family. Protein folding of the peptidase unit for members of this family resembles that of subtilisin, having the clan type SB.

In one embodiment, the protease used according to a process described herein is a Cysteine proteases.

In one embodiment, the protease used according to a process described herein is a Aspartic proteases. Aspartic acid proteases are described in, for example, Hand-book of Proteolytic En-zymes, Edited by A. J. Barrett, N. D. Rawlings and J. F. Woessner, Aca-demic Press, San Diego, 1998, Chapter 270). Suitable examples of aspartic acid protease include, e.g., those disclosed in R. M. Berka et al. Gene, 96, 313 (1990)); (R. M. Berka et al. Gene, 125, 195-198 (1993)); and Gomi et al. Biosci. Biotech. Biochem. 57, 1095-1100 (1993), which are hereby incorporated by reference.

The protease also may be a metalloprotease, which is defined as a protease selected from the group consisting of:

(a) proteases belonging to EC 3.4.24 (metalloendopeptidases); preferably EC 3.4.24.39 (acid metallo proteinases);

(b) metalloproteases belonging to the M group of the above Handbook;

(c) metalloproteases not yet assigned to clans (designation: Clan MX), or belonging to either one of clans MA, MB, MC, MD, ME, MF, MG, MH (as defined at pp. 989-991 of the above Handbook);

(d) other families of metalloproteases (as defined at pp. 1448-1452 of the above Handbook);

(e) metalloproteases with a HEXXH motif;

(f) metalloproteases with an HEFTH motif;

(g) metalloproteases belonging to either one of families M3, M26, M27, M32, M34, M35, M36, M41, M43, or M47 (as defined at pp. 1448-1452 of the above Handbook);

(h) metalloproteases belonging to the M28E family; and

(i) metalloproteases belonging to family M35 (as defined at pp. 1492-1495 of the above Handbook).

In other particular embodiments, metalloproteases are hydrolases in which the nucleophilic attack on a peptide bond is mediated by a water molecule, which is activated by a divalent metal cation. Examples of divalent cations are zinc, cobalt or manganese. The metal ion may be held in place by amino acid ligands. The number of ligands may be five, four, three, two, one or zero. In a particular embodiment the number is two or three, preferably three.

There are no limitations on the origin of the metalloprotease used in a process of the invention. In an embodiment the metalloprotease is classified as EC 3.4.24, preferably EC 3.4.24.39. In one embodiment, the metalloprotease is an acid-stable metalloprotease, e.g., a fungal acid-stable metalloprotease, such as a metalloprotease derived from a strain of the genus Thermoascus, preferably a strain of Thermoascus aurantiacus, especially Thermoascus aurantiacus CGMCC No. 0670 (classified as EC 3.4.24.39). In another embodiment, the metalloprotease is derived from a strain of the genus Aspergillus, preferably a strain of Aspergillus oryzae.

In one embodiment the metalloprotease has a degree of sequence identity to amino acids −178 to 177, −159 to 177, or preferably amino acids 1 to 177 (the mature polypeptide) of SEQ ID NO: 1 of WO2010/008841 (a Thermoascus aurantiacus metalloprotease) of at least 80%, at least 82%, at least 85%, at least 90%, at least 95%, or at least 97%; and which have metalloprotease activity. In particular embodiments, the metalloprotease consists of an amino acid sequence with a degree of identity to SEQ ID NO: 1 as mentioned above.

The Thermoascus aurantiacus metalloprotease is a preferred example of a metalloprotease suitable for use in a process of the invention. Another metalloprotease is derived from Aspergillus oryzae and comprises the sequence of SEQ ID NO: 11 disclosed in WO2003/048353, or amino acids −23-353; −23-374; −23-397; 1-353; 1-374; 1-397; 177-353; 177-374; or 177-397 thereof, and SEQ ID NO: 10 disclosed in WO2003/048353.

Another metalloprotease suitable for use in a process of the invention is the Aspergillus oryzae metalloprotease comprising SEQ ID NO: 5 of WO2010/008841, or a metalloprotease is an isolated polypeptide which has a degree of identity to SEQ ID NO: 5 of at least about 80%, at least 82%, at least 85%, at least 90%, at least 95%, or at least 97%; and which have metalloprotease activity. In particular embodiments, the metalloprotease consists of the amino acid sequence of SEQ ID NO: 5 of WO2010/008841.

In a particular embodiment, a metalloprotease has an amino acid sequence that differs by forty, thirty-five, thirty, twenty-five, twenty, or by fifteen amino acids from amino acids −178 to 177, −159 to 177, or +1 to 177 of the amino acid sequences of the Thermoascus aurantiacus or Aspergillus oryzae metalloprotease.

In another embodiment, a metalloprotease has an amino acid sequence that differs by ten, or by nine, or by eight, or by seven, or by six, or by five amino acids from amino acids −178 to 177, −159 to 177, or +1 to 177 of the amino acid sequences of these metalloproteases, e.g., by four, by three, by two, or by one amino acid.

In particular embodiments, the metalloprotease a) comprises or b) consists of

i) the amino acid sequence of amino acids −178 to 177, −159 to 177, or +1 to 177 of SEQ ID NO:1 of WO2010/008841;

ii) the amino acid sequence of amino acids −23-353, −23-374, −23-397, 1-353, 1-374, 1-397, 177-353, 177-374, or 177-397 of SEQ ID NO: 3 of WO2010/008841;

iii) the amino acid sequence of SEQ ID NO: 5 of WO2010/008841; or allelic variants, or fragments, of the sequences of i), ii), and iii) that have protease activity.

A fragment of amino acids −178 to 177, −159 to 177, or +1 to 177 of SEQ ID NO: 1 of WO02010/008841 or of amino acids −23-353, −23-374, −23-397, 1-353, 1-374, 1-397, 177-353, 177-374, or 177-397 of SEQ ID NO: 3 of WO2010/008841; is a polypeptide having one or more amino acids deleted from the amino and/or carboxyl terminus of these amino acid sequences. In one embodiment a fragment contains at least 75 amino acid residues, or at least 100 amino acid residues, or at least 125 amino acid residues, or at least 150 amino acid residues, or at least 160 amino acid residues, or at least 165 amino acid residues, or at least 170 amino acid residues, or at least 175 amino acid residues.

To determine whether a given protease is a metallo protease or not, reference is made to the above “Handbook of Proteolytic Enzymes” and the principles indicated therein. Such determination can be carried out for all types of proteases, be it naturally occurring or wild-type proteases; or genetically engineered or synthetic proteases.

The protease may be a variant of, e.g., a wild-type protease, having thermostability properties defined herein. In one embodiment, the thermostable protease is a variant of a metallo protease. In one embodiment, the thermostable protease used in a process described herein is of fungal origin, such as a fungal metallo protease, such as a fungal metallo protease derived from a strain of the genus Thermoascus, preferably a strain of Thermoascus aurantiacus, especially Thermoascus aurantiacus CGMCC No. 0670 (classified as EC 3.4.24.39).

In one embodiment, the thermostable protease is a variant of the mature part of the metallo protease shown in SEQ ID NO: 2 disclosed in WO2003/048353 or the mature part of SEQ ID NO: 1 in WO2010/008841 further with one of the following substitutions or combinations of substitutions:

S5*+D79L+S87P+A112P+D142L;

D79L+S87P+A112P+T124V+D142L;

S5*+N26R+D79L+S87P+A112P+D142L;

N26R+T46R+D79L+S87P+A112P+D142L;

T46R+D79L+S87P+T116V+D142L;

D79L+P81R+S87P+A112P+D142L;

A27K+D79L+S87P+A112P+T124V+D142L;

D79L+Y82F+S87P+A112P+T124V+D142L;

D79L+Y82F+S87P+A112P+T124V+D142L;

D79L+S87P+A112P+T124V+A126V+D142L;

D79L+S87P+A112P+D142L;

D79L+Y82F+S87P+A112P+D142L;

S38T+D79L+S87P+A112P+A126V+D142L;

D79L+Y82F+S87P+A112P+A126V+D142L;

A27K+D79L+S87P+A112P+A126V+D142L;

D79L+S87P+N98C+A112P+G135C+D142L;

D79L+S87P+A112P+D142L+T141C+M161C;

S36P+D79L+S87P+A112P+D142L;

A37P+D79L+S87P+A112P+D142L;

S49P+D79L+S87P+A112P+D142L;

S50P+D79L+S87P+A112P+D142L;

D79L+S87P+D104P+A112P+D142L;

D79L+Y82F+S87G+A112P+D142L;

S70V+D79L+Y82F+S87G+Y97W+A112P+D142L;

D79L+Y82F+S87G+Y97W+D104P+A112P+D142L;

S70V+D79L+Y82F+S87G+A112P+D142L;

D79L+Y82F+S87G+D104P+A112P+D142L;

D79L+Y82F+S87G+A112P+A126V+D142L;

Y82F+S87G+S70V+D79L+D104P+A112P+D142L;

Y82F+S87G+D79L+D104P+A112P+A126V+D142L;

A27K+D79L+Y82F+S87G+D104P+A112P+A126V+D142L;

A27K+Y82F+S87G+D104P+A112P+A126V+D142L;

A27K+D79L+Y82F+D104P+A112P+A126V+D142L;

A27K+Y82F+D104P+A112P+A126V+D142L;

A27K+D79L+S87P+A112P+D142L; and

D79L+S87P+D142L.

In one embodiment, the thermostable protease is a variant of the metallo protease disclosed as the mature part of SEQ ID NO: 2 disclosed in WO2003/048353 or the mature part of SEQ ID NO: 1 in WO02010/008841 with one of the following substitutions or combinations of substitutions:

D79L+S87P+A112P+D142L;

D79L+S87P+D142L; and

A27K+D79L+Y82F+S87G+D104P+A112P+A126V+D142L.

In one embodiment, the protease variant has at least 75% identity preferably at least 80%, more preferably at least 85%, more preferably at least 90%, more preferably at least 91%, more preferably at least 92%, even more preferably at least 93%, most preferably at least 94%, and even most preferably at least 95%, such as even at least 96%, at least 97%, at least 98%, at least 99%, but less than 100% identity to the mature part of the polypeptide of SEQ ID NO: 2 disclosed in WO2003/048353 or the mature part of SEQ ID NO: 1 in WO2010/008841.

The thermostable protease may also be derived from any bacterium as long as the protease has the thermostability properties.

In one embodiment, the thermostable protease is derived from a strain of the bacterium Pyrococcus, such as a strain of Pyrococcus furiosus (pfu protease).

In one embodiment, the protease is one shown as SEQ ID NO: 1 in U.S. Pat. No. 6,358,726-B1 (Takara Shuzo Company).

In one embodiment, the thermostable protease is a protease having a mature polypeptide sequence of at least 80% identity, such as at least 85%, such as at least 90%, such as at least 95%, such as at least 96%, such as at least 97%, such as at least 98%, such as at least 99% identity to SEQ ID NO: 1 in U.S. Pat. No. 6,358,726-B1. The Pyroccus furiosus protease can be purchased from Takara Bio, Japan.

The Pyrococcus furiosus protease may be a thermostable protease as described in SEQ ID NO: 13 of WO2018/098381. This protease (PfuS) was found to have a thermostability of 110% (80° C./70° C.) and 103% (90° C./70° C.) at pH 4.5 determined.

In one embodiment a thermostable protease used in a process described herein has a thermostability value of more than 20% determined as Relative Activity at 80° C./70° C. determined as described in Example 2 of WO2018098381.

In one embodiment, the protease has a thermostability of more than 30%, more than 40%, more than 50%, more than 60%, more than 70%, more than 80%, more than 90%, more than 100%, such as more than 105%, such as more than 110%, such as more than 115%, such as more than 120% determined as Relative Activity at 80° C./70° C.

In one embodiment, protease has a thermostability of between 20 and 50%, such as between 20 and 40%, such as 20 and 30% determined as Relative Activity at 80° C./70° C. In one embodiment, the protease has a thermostability between 50 and 115%, such as between 50 and 70%, such as between 50 and 60%, such as between 100 and 120%, such as between 105 and 115% determined as Relative Activity at 80° C./70° C.

In one embodiment, the protease has a thermostability value of more than 10% determined as Relative Activity at 85° C./70° C. determined as described in Example 2 of WO2018/098381.

In one embodiment, the protease has a thermostability of more than 10%, such as more than 12%, more than 14%, more than 16%, more than 18%, more than 20%, more than 30%, more than 40%, more that 50%, more than 60%, more than 70%, more than 80%, more than 90%, more than 100%, more than 110% determined as Relative Activity at 85° C./70° C.

In one embodiment, the protease has a thermostability of between 10% and 50%, such as between 10% and 30%, such as between 10% and 25% determined as Relative Activity at 85° C./70° C.

In one embodiment, the protease has more than 20%, more than 30%, more than 40%, more than 50%, more than 60%, more than 70%, more than 80%, more than 90% determined as Remaining Activity at 80° C.; and/or the protease has more than 20%, more than 30%, more than 40%, more than 50%, more than 60%, more than 70%, more than 80%, more than 90% determined as Remaining Activity at 84° C.

Determination of “Relative Activity” and “Remaining Activity” is done as described in Example 2 of WO2018/098381.

In one embodiment, the protease may have a thermostability for above 90, such as above 100 at 85° C. as determined using the Zein-BCA assay as disclosed in Example 3 of WO2018/098381.

In one embodiment, the protease has a thermostability above 60%, such as above 90%, such as above 100%, such as above 110% at 85° C. as determined using the Zein-BCA assay of WO2018/098381.

In one embodiment, protease has a thermostability between 60-120, such as between 70-120%, such as between 80-120%, such as between 90-120%, such as between 100-120%, such as 110-120% at 85° C. as determined using the Zein-BCA assay of WO2018/098381.

In one embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the activity of the JTP196 protease variant or Protease Pfu determined by the AZCL-casein assay of WO2018/098381, and described herein.

In one embodiment, the thermostable protease has at least 20%, such as at least 30%, such as at least 40%, such as at least 50%, such as at least 60%, such as at least 70%, such as at least 80%, such as at least 90%, such as at least 95%, such as at least 100% of the protease activity of the Protease 196 variant or Protease Pfu determined by the AZCL-casein assay of WO2018/098381.

### Pullulanases

The host cells and fermenting organisms may express a heterologous pullulanase. The pullulanase can be any protease that is suitable for the host cells and fermenting organisms and/or their methods of use described herein, such as a naturally occurring pullulanase or a variant thereof that retains pullulanase activity. Any pullulanase contemplated for expression by a host cell or fermenting organism described below is also contemplated for embodiments of the invention involving exogenous addition of a pullulanase (e.g., added before, during or after liquefaction and/or saccharification).

In some embodiments, the host cell or fermenting organism comprising a heterologous polynucleotide encoding a pullulanase has an increased level of pullulanase activity compared to the host cells without the heterologous polynucleotide encoding the pullulanase, when cultivated under the same conditions. In some embodiments, the host cell or fermenting organism has an increased level of pullulanase activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the pullulanase, when cultivated under the same conditions.

Exemplary pullulanases that can be used with the host cells and/or the methods described herein include bacterial, yeast, or filamentous fungal pullulanases, e.g., obtained from any of the microorganisms described or referenced herein.

Contemplated pullulanases include the pullulanases from Bacillus amyloderamificans disclosed in U.S. Pat. No. 4,560,651 (hereby incorporated by reference), the pullulanase disclosed as SEQ ID NO: 2 in WO 01/151620 (hereby incorporated by reference), the Bacillus deramificans disclosed as SEQ ID NO: 4 in WO 01/151620 (hereby incorporated by reference), and the pullulanase from Bacillus acidopullulyticus disclosed as SEQ ID NO: 6 in WO 01/151620 (hereby incorporated by reference) and also described in FEMS Mic. Let. (1994) 115, 97-106.

Additional pullulanases contemplated include the pullulanases from Pyrococcus woesei, specifically from Pyrococcus woesei DSM No. 3773 disclosed in WO92/02614.

In one embodiment, the pullulanase is a family GH57 pullulanase. In one embodiment, the pullulanase includes an X47 domain as disclosed in U.S. 61/289,040 published as WO02011/087836 (which are hereby incorporated by reference). More specifically the pullulanase may be derived from a strain of the genus Thermococcus, including Thermococcus litoralis and Thermococcus hydrothermalis, such as the Thermococcus hydrothermalis pullulanase truncated at site X4 right after the X47 domain (i.e., amino acids 1-782). The pullulanase may also be a hybrid of the Thermococcus litoralis and Thermococcus hydrothermalis pullulanases or a T. hydrothermalis/T. litoralis hybrid enzyme with truncation site X4 disclosed in U.S. 61/289,040 published as WO02011/087836 (which is hereby incorporated by reference).

In another embodiment, the pullulanase is one comprising an X46 domain disclosed in WO02011/076123 (Novozymes).

The pullulanase may be added in an effective amount which include the preferred amount of about 0.0001-10 mg enzyme protein per gram DS, preferably 0.0001-0.10 mg enzyme protein per gram DS, more preferably 0.0001-0.010 mg enzyme protein per gram DS.

Pullulanase activity may be determined as NPUN. An Assay for determination of NPUN is described in WO2018/098381.

Suitable commercially available pullulanase products include PROMOZYME D, PROMOZYME™ D2 (Novozymes A/S, Denmark), OPTIMAX L-300 (DuPont-Danisco, USA), and AMANO 8 (Amano, Japan).

In one embodiment, the pullulanase is derived from the Bacillus subtilis pullulanase of SEQ ID NO: 114. In one embodiment, the pullulanase is derived from the Bacillus licheniformis pullulanase of SEQ ID NO: 115. In one embodiment, the pullulanase is derived from the Oryza sativa pullulanase of SEQ ID NO: 116. In one embodiment, the pullulanase is derived from the Triticum aestivum pullulanase of SEQ ID NO: 117. In one embodiment, the pullulanase is derived from the Clostridium phytofermentans pullulanase of SEQ ID NO: 118. In one embodiment, the pullulanase is derived from the Streptomyces avermitilis pullulanase of SEQ ID NO: 119. In one embodiment, the pullulanase is derived from the Klebsiella pneumoniae pullulanase of SEQ ID NO: 120.

Additional pullulanases contemplated for use with the present invention can be found in WO2011/153516 (the content of which is incorporated herein).

Additional polynucleotides encoding suitable pullulanases may be obtained from microorganisms of any genus, including those readily available within the UniProtKB database.

The pullulanase coding sequences can also be used to design nucleic acid probes to identify and clone DNA encoding pullulanases from strains of different genera or species, as described supra.

The polynucleotides encoding pullulanases may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding pullulanases are described supra.

In one embodiment, the pullulanase has a mature polypeptide sequence that comprises or consists of the amino acid sequence of any one of the pullulanases described or referenced herein (e.g., any one of SEQ ID NOs: 114-120). In another embodiment, the pullulanase has a mature polypeptide sequence that is a fragment of the any one of the pullulanases described or referenced herein (e.g., any one of SEQ ID NOs: 114-120). In one embodiment, the number of amino acid residues in the fragment is at least 75%, e.g., at least 80%, 85%, 90%, or 95% of the number of amino acid residues in referenced full length pullulanase. In other embodiments, the pullulanase may comprise the catalytic domain of any pullulanase described or referenced herein (e.g., any one of SEQ ID NOs: 114-120).

The pullulanase may be a variant of any one of the pullulanases described supra (e.g., any one of SEQ ID NOs: 114-120). In one embodiment, the pullulanase has a mature polypeptide sequence of at least 60%, e.g., at least 65%, 70%, 75%, 80%, 85%, 90%, 95%, 97%, 98%, 99%, or 100% sequence identity to any one of the pullulanases described supra (e.g., any one of SEQ ID NOs: 114-120).

Examples of suitable amino acid changes, such as conservative substitutions that do not significantly affect the folding and/or activity of the pullulanase, are described herein.

In one embodiment, the pullulanase has a mature polypeptide sequence that differs by no more than ten amino acids, e.g., by no more than five amino acids, by no more than four amino acids, by no more than three amino acids, by no more than two amino acids, or by one amino acid from the amino acid sequence of any one of the pullulanases described supra (e.g., any one of SEQ ID NOs: 114-120). In one embodiment, the pullulanase has an amino acid substitution, deletion, and/or insertion of one or more (e.g., two, several) of amino acid sequence of any one of the pullulanases described supra (e.g., any one of SEQ ID NOs: 114-120). In some embodiments, the total number of amino acid substitutions, deletions and/or insertions is not more than 10, e.g., not more than 9, 8, 7, 6, 5, 4, 3, 2, or 1.

In some embodiments, the pullulanase has at least 20%, e.g., at least 40%, at least 50%, at least 60%, at least 70%, at least 80%, at least 90%, at least 95%, at least 96%, at least 97%, at least 98%, at least 99%, or 100% of the pullulanase activity of any pullulanase described or referenced herein under the same conditions (e.g., any one of SEQ ID NOs: 114-120).

In one embodiment, the pullulanase coding sequence hybridizes under at least low stringency conditions, e.g., medium stringency conditions, medium-high stringency conditions, high stringency conditions, or very high stringency conditions with the full-length complementary strand of the coding sequence from any pullulanase described or referenced herein (e.g., any one of SEQ ID NOs: 114-120). In one embodiment, the pullulanase coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any pullulanase described or referenced herein (e.g., any one of SEQ ID NOs: 114-120).

In one embodiment, the pullulanase comprises the coding sequence of any pullulanase described or referenced herein (e.g., any one of SEQ ID NOs: 114-120). In one embodiment, the pullulanase comprises a coding sequence that is a subsequence of the coding sequence from any pullulanase described or referenced herein, wherein the subsequence encodes a polypeptide having pullulanase activity. In one embodiment, the number of nucleotides residues in the subsequence is at least 75%, e.g., at least 80%, 85%, 90%, or 95% of the number of the referenced coding sequence.

The referenced pullulanase coding sequence of any related aspect or embodiment described herein can be the native coding sequence or a degenerate sequence, such as a codon-optimized coding sequence designed for use in a particular host cell (e.g., optimized for expression in Saccharomyces cerevisiae).

The pullulanase can also include fused polypeptides or cleavable fusion polypeptides, as described supra. In some embodiments, the pullulanase is a fusion protein comprising a signal peptide linked to the N-terminus of a mature polypeptide, such as any signal sequences described in U.S. Provisional Application No. 62/883,519 filed Aug. 6, 2019 and entitled “Fusion Proteins For Improved Enzyme Expression” (the content of which is hereby incorporated by reference).

### Gene Disruptions

The host cells and fermenting organisms described herein may also comprise one or more (e.g., two, several) gene disruptions, e.g., to divert sugar metabolism from undesired products to ethanol. In some embodiments, the recombinant host cells produce a greater amount of ethanol compared to the cell without the one or more disruptions when cultivated under identical conditions. In some embodiments, one or more of the disrupted endogenous genes is inactivated. In some embodiments, the host cell or fermenting organism is a diploid and has a disruption (e.g., inactivation) of both copies of the referenced gene.

In certain embodiments, the host cell or fermenting organism provided herein comprises a disruption of one or more endogenous genes encoding enzymes involved in producing alternate fermentative products such as glycerol or other byproducts such as acetate or diols. For example, the cells provided herein may comprise a disruption of one or more endogenous genes encoding a glycerol 3-phosphatase (GPP, E.C. 3.1.3.21, catalyzes conversion of glycerol-3 phosphate to glycerol), a glycerol 3-phosphate dehydrogenase (GPD, catalyzes reaction of dihydroxyacetone phosphate to glycerol 3-phosphate), glycerol kinase (catalyzes conversion of glycerol 3-phosphate to glycerol), dihydroxyacetone kinase (catalyzes conversion of dihydroxyacetone phosphate to dihydroxyacetone), glycerol dehydrogenase (catalyzes conversion of dihydroxyacetone to glycerol), and aldehyde dehydrogenase (ALD, e.g., converts acetaldehyde to acetate).

In some embodiments, the host cell or fermenting organism comprises a disruption to one or more endogenous genes encoding a glycerol 3-phosphatase (GPP). Saccharomyces cerevisiae has two glycerol-3-phosphate phosphatase paralogs encoding GPP1 (UniProt No. P41277; SEQ ID NO: 257) and GPP2 (UniProt No. P40106; SEQ ID NO: 258) (Pahlman et al. (2001) J. Biol. Chem. 276(5):3555-63; Norbeck et al. (1996) J. Biol. Chem. 271(23):13875-81). In some embodiments, the host cell or fermenting organism comprises a disruption to GPP1. In some embodiments, the host cell or fermenting organism comprises a disruption to GPP2. In some embodiments, the host cell or fermenting organism comprises a disruption to GPP1 and GPP2.

In some embodiments, the host cell or fermenting organism comprises a disruption to one or more endogenous genes encoding a glycerol 3-phosphate dehydrogenase (GPD). Saccharomyces cerevisiae has two glycerol 3-phosphate dehydrogenases which encode GPD1 (UniProt No. Q00055; SEQ ID NO: 259) and GPD2 (UniProt No. P41911; SEQ ID NO: 260). In some embodiments, the host cell or fermenting organism comprises a disruption to GPD1. In some embodiments, the host cell or fermenting organism comprises a disruption to GPD2. In some embodiments, the host cell or fermenting organism comprises a disruption to GPD1 and GPD2.

In some embodiments, the host cell or fermenting organism comprises a disruption to an endogenous gene encoding GPP (e.g., GPP1 and/or GPP2) and/or a GPD (GPD1 and/or GPD2), wherein the host cell or fermenting organism produces a decreased amount of glycerol (e.g., at least 25% less, at least 50% less, at least 60% less, at least 70% less, at least 80% less, or at least 90% less) compared to the cell without the disruption to the endogenous gene encoding the GPP and/or GPD when cultivated under identical conditions.

Modeling analysis can be used to design gene disruptions that additionally optimize utilization of the pathway. One exemplary computational method for identifying and designing metabolic alterations favoring biosynthesis of a desired product is the OptKnock computational framework, Burgard et al., 2003, Biotechnol. Bioeng. 84: 647-657.

The host cells and fermenting organisms comprising a gene disruption may be constructed using methods well known in the art, including those methods described herein. A portion of the gene can be disrupted such as the coding region or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, but are not limited to, a leader, propeptide sequence, signal sequence, transcription terminator, and transcriptional activator.

The host cells and fermenting organisms comprising a gene disruption may be constructed by gene deletion techniques to eliminate or reduce expression of the gene. Gene deletion techniques enable the partial or complete removal of the gene thereby eliminating their expression. In such methods, deletion of the gene is accomplished by homologous recombination using a plasmid that has been constructed to contiguously contain the 5′ and 3′ regions flanking the gene.

The host cells and fermenting organisms comprising a gene disruption may also be constructed by introducing, substituting, and/or removing one or more (e.g., two, several) nucleotides in the gene or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, 1985, Science 229: 4719; Lo et al., 1985, Proc. Natl. Acad. Sci. U.S.A. 81: 2285; Higuchi et al., 1988, Nucleic Acids Res 16: 7351; Shimada, 1996, Meth. Mol. Biol. 57:157; Ho et al., 1989, Gene 77: 61; Horton et al., 1989, Gene 77: 61; and Sarkar and Sommer, 1990, BioTechniques 8: 404.

The host cells and fermenting organisms comprising a gene disruption may also be constructed by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The host cells and fermenting organisms comprising a gene disruption may also be constructed by the process of gene conversion (see, for example, Iglesias and Trautner, 1983, Molecular General Genetics 189: 73-76). For example, in the gene conversion method, a nucleotide sequence corresponding to the gene is mutagenized in vitro to produce a defective nucleotide sequence, which is then transformed into the recombinant strain to produce a defective gene. By homologous recombination, the defective nucleotide sequence replaces the endogenous gene. It may be desirable that the defective nucleotide sequence also comprises a marker for selection of transformants containing the defective gene.

The host cells and fermenting organisms comprising a gene disruption may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the gene may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. Men such agents are used, the mutagenesis is typically performed by incubating the parent strain to be mutagenized in the presence of the mutagenizing agent of choice under suitable conditions, and selecting for mutants exhibiting reduced or no expression of the gene.

A nucleotide sequence homologous or complementary to a gene described herein may be used from other microbial sources to disrupt the corresponding gene in a recombinant strain of choice.

In one embodiment, the modification of a gene in the recombinant cell is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

### Methods Using a Starch-Containing Material

In some embodiments, the methods described herein produce a fermentation product from a starch-containing material. Starch-containing material is well-known in the art, containing two types of homopolysaccharides (amylose and amylopectin) and is linked by alpha-(1-4)-D-glycosidic bonds. Any suitable starch-containing starting material may be used. The starting material is generally selected based on the desired fermentation product, such as ethanol. Examples of starch-containing starting materials include cereal, tubers or grains. Specifically, the starch-containing material may be corn, wheat, barley, rye, milo, sago, cassava, tapioca, sorghum, oat, rice, peas, beans, or sweet potatoes, or mixtures thereof. Contemplated are also waxy and non-waxy types of corn and barley.

In one embodiment, the starch-containing starting material is corn. In one embodiment, the starch-containing starting material is wheat. In one embodiment, the starch-containing starting material is barley. In one embodiment, the starch-containing starting material is rye. In one embodiment, the starch-containing starting material is milo. In one embodiment, the starch-containing starting material is sago. In one embodiment, the starch-containing starting material is cassava. In one embodiment, the starch-containing starting material is tapioca. In one embodiment, the starch-containing starting material is sorghum. In one embodiment, the starch-containing starting material is rice. In one embodiment, the starch-containing starting material is peas. In one embodiment, the starch-containing starting material is beans. In one embodiment, the starch-containing starting material is sweet potatoes. In one embodiment, the starch-containing starting material is oats.

The methods using a starch-containing material may include a conventional process (e.g., including a liquefaction step described in more detail below) or a raw starch hydrolysis process. In some embodiments using a starch-containing material, saccharification of the starch-containing material is at a temperature above the initial gelatinization temperature. In some embodiments using a starch-containing material, saccharification of the starch-containing material is at a temperature below the initial gelatinization temperature.

### Liquefaction

In embodiments using a starch-containing material, the methods may further comprise a liquefaction step carried out by subjecting the starch-containing material at a temperature above the initial gelatinization temperature to an alpha-amylase and optionally a protease and/or a glucoamylase. Other enzymes such as a pullulanase and phytase may also be present and/or added in liquefaction. In some embodiments, the liquefaction step is carried out prior to steps a) and b) of the described methods.

Liquefaction step may be carried out for 0.5-5 hours, such as 1-3 hours, such as typically about 2 hours.

The term “initial gelatinization temperature” means the lowest temperature at which gelatinization of the starch-containing material commences. In general, starch heated in water begins to gelatinize between about 50° C. and 75° C.; the exact temperature of gelatinization depends on the specific starch and can readily be determined by the skilled artisan. Thus, the initial gelatinization temperature may vary according to the plant species, to the particular variety of the plant species as well as with the growth conditions. The initial gelatinization temperature of a given starch-containing material may be determined as the temperature at which birefringence is lost in 5% of the starch granules using the method described by Gorinstein and Lii, 1992, Starch/Starke 44(12): 461-466.

Liquefaction is typically carried out at a temperature in the range from 70-100° C. In one embodiment, the temperature in liquefaction is between 75-95° C., such as between 75-90° C., between 80-90° C., or between 82-88° C., such as about 85° C.

A jet-cooking step may be carried out prior to liquefaction in step, for example, at a temperature between 110-145° C., 120-140° C., 125-135° C., or about 130° C. for about 1-15 minutes, for about 3-10 minutes, or about 5 minutes.

The pH during liquefaction may be between 4 and 7, such as pH 4.5-6.5, pH 5.0-6.5, pH 5.0-6.0, pH 5.2-6.2, or about 5.2, about 5.4, about 5.6, or about 5.8.

In one embodiment, the process further comprises, prior to liquefaction, the steps of:

i) reducing the particle size of the starch-containing material, preferably by dry milling;

ii) forming a slurry comprising the starch-containing material and water.

The starch-containing starting material, such as whole grains, may be reduced in particle size, e.g., by milling, in order to open up the structure, to increase surface area, and allowing for further processing. Generally, there are two types of processes: wet and dry milling. In dry milling whole kernels are milled and used. Wet milling gives a good separation of germ and meal (starch granules and protein). Wet milling is often applied at locations where the starch hydrolysate is used in production of, e.g., syrups. Both dry milling and wet milling are well known in the art of starch processing. In one embodiment the starch-containing material is subjected to dry milling. In one embodiment, the particle size is reduced to between 0.05 to 3.0 mm, e.g., 0.1-0.5 mm, or so that at least 30%, at least 50%, at least 70%, or at least 90% of the starch-containing material fit through a sieve with a 0.05 to 3.0 mm screen, e.g., 0.1-0.5 mm screen. In another embodiment, at least 50%, e.g., at least 70%, at least 80%, or at least 90% of the starch-containing material fit through a sieve with #6 screen.

The aqueous slurry may contain from 10-55 w/w-% dry solids (DS), e.g., 25-45 w/w-% dry solids (DS), or 30-40 w/w-% dry solids (DS) of starch-containing material.

The alpha-amylase, optionally a protease, and optionally a glucoamylase may initially be added to the aqueous slurry to initiate liquefaction (thinning). In one embodiment, only a portion of the enzymes (e.g., about ⅓) is added to the aqueous slurry, while the rest of the enzymes (e.g., about ⅔) are added during liquefaction step.

A non-exhaustive list of alpha-amylases used in liquefaction can be found in the “Alpha-Amylases” section. Examples of suitable proteases used in liquefaction include any protease described supra in the “Proteases” section. Examples of suitable glucoamylases used in liquefaction include any glucoamylase found in the “Glucoamylases” section.

### Saccharification and Fermentation of Starch-Containing Material

In embodiments using a starch-containing material, a glucoamylase may be present and/or added in saccharification step a) and/or fermentation step b) or simultaneous saccharification and fermentation (SSF). The glucoamylase of the saccharification step a) and/or fermentation step b) or simultaneous saccharification and fermentation (SSF) is typically different from the glucoamylase optionally added to any liquefaction step described supra. In one embodiment, the glucoamylase is present and/or added together with a fungal alpha-amylase.

In some embodiments, the host cell or fermenting organism comprises a heterologous polynucleotide encoding a glucoamylase, for example, as described in WO2017/087330, the content of which is hereby incorporated by reference.

Examples of glucoamylases can be found in the “Glucoamylases” section.

When doing sequential saccharification and fermentation, saccharification step a) may be carried out under conditions well-known in the art. For instance, saccharification step a) may last up to from about 24 to about 72 hours. In one embodiment, pre-saccharification is done. Pre-saccharification is typically done for 40-90 minutes at a temperature between 30-65° C., typically about 60° C. Pre-saccharification is, in one embodiment, followed by saccharification during fermentation in simultaneous saccharification and fermentation (SSF). Saccharification is typically carried out at temperatures from 20-75° C., preferably from 40-70° C., typically about 60° C., and typically at a pH between 4 and 5, such as about pH 4.5.

Fermentation is carried out in a fermentation medium, as known in the art and, e.g., as described herein. The fermentation medium includes the fermentation substrate, that is, the carbohydrate source that is metabolized by the fermenting organism. With the processes described herein, the fermentation medium may comprise nutrients and growth stimulator(s) for the fermenting organism(s). Nutrient and growth stimulators are widely used in the art of fermentation and include nitrogen sources, such as ammonia; urea, vitamins and minerals, or combinations thereof.

Generally, fermenting organisms such as yeast, including Saccharomyces cerevisiae yeast, require an adequate source of nitrogen for propagation and fermentation. Many sources of supplemental nitrogen, if necessary, can be used and such sources of nitrogen are well known in the art. The nitrogen source may be organic, such as urea, DDGs, wet cake or corn mash, or inorganic, such as ammonia or ammonium hydroxide. In one embodiment, the nitrogen source is urea.

Fermentation can be carried out under low nitrogen conditions, e.g., when using a protease-expressing yeast. In some embodiments, the fermentation step is conducted with less than 1000 ppm supplemental nitrogen (e.g., urea or ammonium hydroxide), such as less than 750 ppm, less than 500 ppm, less than 400 ppm, less than 300 ppm, less than 250 ppm, less than 200 ppm, less than 150 ppm, less than 100 ppm, less than 75 ppm, less than 50 ppm, less than 25 ppm, or less than 10 ppm, supplemental nitrogen. In some embodiments, the fermentation step is conducted with no supplemental nitrogen.

Simultaneous saccharification and fermentation (“SSF”) is widely used in industrial scale fermentation product production processes, especially ethanol production processes. When doing SSF the saccharification step a) and the fermentation step b) are carried out simultaneously. There is no holding stage for the saccharification, meaning that a fermenting organism, such as yeast, and enzyme(s), may be added together. However, it is also contemplated to add the fermenting organism and enzyme(s) separately. SSF is typically carried out at a temperature from 25° C. to 40° C., such as from 28° C. to 35° C., such as from 30° C. to 34° C., or about 32° C. In one embodiment, fermentation is ongoing for 6 to 120 hours, in particular 24 to 96 hours. In one embodiment, the pH is between 4-5.

In one embodiment, a cellulolytic enzyme composition is present and/or added in saccharification, fermentation or simultaneous saccharification and fermentation (SSF). Examples of such cellulolytic enzyme compositions can be found in the “Cellulolytic Enzymes and Compositions” section. The cellulolytic enzyme composition may be present and/or added together with a glucoamylase, such as one disclosed in the “Glucoamylases” section.

### Methods Using a Cellulosic-Containing Material

In some embodiments, the methods described herein produce a fermentation product from a cellulosic-containing material. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic-containing material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In one embodiment, the cellulosic-containing material is any biomass material. In another embodiment, the cellulosic-containing material is lignocellulose, which comprises cellulose, hemicelluloses, and lignin.

In one embodiment, the cellulosic-containing material is agricultural residue, herbaceous material (including energy crops), municipal solid waste, pulp and paper mill residue, waste paper, or wood (including forestry residue).

In another embodiment, the cellulosic-containing material is arundo, bagasse, bamboo, corn cob, corn fiber, corn stover, miscanthus, rice straw, switchgrass, or wheat straw.

In another embodiment, the cellulosic-containing material is aspen, eucalyptus, fir, pine, poplar, spruce, or willow.

In another embodiment, the cellulosic-containing material is algal cellulose, bacterial cellulose, cotton linter, filter paper, microcrystalline cellulose (e.g., AVICEL®), or phosphoric-acid treated cellulose.

In another embodiment, the cellulosic-containing material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic-containing material may be used as is or may be subjected to pretreatment, using conventional methods known in the art, as described herein. In a preferred embodiment, the cellulosic-containing material is pretreated.

The methods of using cellulosic-containing material can be accomplished using methods conventional in the art. Moreover, the methods of can be implemented using any conventional biomass processing apparatus configured to carry out the processes.

### Cellulosic Pretreatment

In one embodiment the cellulosic-containing material is pretreated before saccharification.

In practicing the processes described herein, any pretreatment process known in the art can be used to disrupt plant cell wall components of the cellulosic-containing material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9:1621-1651; Yang and Wyman, 2008, Biofuels Bioproducts and Biorefning-Biofpr. 2: 26-40).

The cellulosic-containing material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

In a one embodiment, the cellulosic-containing material is pretreated before saccharification (i.e., hydrolysis) and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

In one embodiment, the cellulosic-containing material is pretreated with steam. In steam pretreatment, the cellulosic-containing material is heated to disrupt the plant cell wall components, including lignin, hemicellulose, and cellulose to make the cellulose and other fractions, e.g., hemicellulose, accessible to enzymes. The cellulosic-containing material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on optional addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and optional addition of a chemical catalyst. Steam pretreatment allows for relatively high solids loadings, so that the cellulosic-containing material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

In one embodiment, the cellulosic-containing material is subjected to a chemical pretreatment. The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A chemical catalyst such as H₂SO₄ or SO₂ (typically 0.3 to 5% w/w) is sometimes added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic-containing material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, Bioresource Technology 855: 1-33; Schell et al., 2004, Bioresource Technology 91: 179-188; Lee et al., 1999, Adv. Biochem. Eng. Biotechnol. 65: 93-115). In a specific embodiment the dilute acid pretreatment of cellulosic-containing material is carried out using 4% w/w sulfuric acid at 180° C. for 5 minutes.

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment. Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673486). WO2006/110891, WO2006/110899, WO2006/110900, and WO02006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic-containing material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic-containing material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. Biotechnol. 105-108: 69-85, and Mosier et al., 2005, Bioresource Technology 96: 673-686, and U.S. Published Application 2002/0164730.

In one embodiment, the chemical pretreatment is carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one embodiment, the acid concentration is in the range from preferably 0.01 to 10 wt. % acid, e.g., 0.05 to 5 wt. % acid or 0.1 to 2 wt. % acid. The acid is contacted with the cellulosic-containing material and held at a temperature in the range of preferably 140-200° C., e.g., 165-190° C., for periods ranging from 1 to 60 minutes.

In another embodiment, pretreatment takes place in an aqueous slurry. In preferred embodiments, the cellulosic-containing material is present during pretreatment in amounts preferably between 10-80 wt. %, e.g., 20-70 wt. % or 30-60 wt. %, such as around 40 wt. %. The pretreated cellulosic-containing material can be unwashed or washed using any method known in the art, e.g., washed with water.

In one embodiment, the cellulosic-containing material is subjected to mechanical or physical pretreatment. The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic-containing material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one embodiment, high pressure means pressure in the range of preferably about 100 to about 400 psi, e.g., about 150 to about 250 psi. In another embodiment, high temperature means temperature in the range of about 100 to about 300° C., e.g., about 140 to about 200° C. In a preferred embodiment, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in one embodiment, the cellulosic-containing material is subjected to physical (mechanical) or chemical pretreatment, or any combination thereof, to promote the separation and/or release of cellulose, hemicellulose, and/or lignin.

In one embodiment, the cellulosic-containing material is subjected to a biological pretreatment. The term “biological pretreatment” refers to any biological pretreatment that promotes the separation and/or release of cellulose, hemicellulose, and/or lignin from the cellulosic-containing material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Saccharification and Fermentation of Cellulosic-Containing Material

Saccharification (i.e., hydrolysis) and fermentation, separate or simultaneous, include, but are not limited to, separate hydrolysis and fermentation (SHF); simultaneous saccharification and fermentation (SSF); simultaneous saccharification and co-fermentation (SSCF); hybrid hydrolysis and fermentation (HHF); separate hydrolysis and co-fermentation (SHCF); hybrid hydrolysis and co-fermentation (HHCF).

SHF uses separate process steps to first enzymatically hydrolyze the cellulosic-containing material to fermentable sugars, e.g., glucose, cellobiose, and pentose monomers, and then ferment the fermentable sugars to ethanol. In SSF, the enzymatic hydrolysis of the cellulosic-containing material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the co-fermentation of multiple sugars (Sheehan and Himmel, 1999, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation organismcan tolerate. It is understood herein that any method known in the art comprising pretreatment, enzymatic hydrolysis (saccharification), fermentation, or a combination thereof, can be used in the practicing the processes described herein.

A conventional apparatus can include a fed-batch stirred reactor, a batch stirred reactor, a continuous flow stirred reactor with ultrafiltration, and/or a continuous plug-flow column reactor (de Castilhos Corazza et al., 2003, Acta Scientiarum. Technology 25: 33-38; Gusakov and Sinitsyn, 1985, Enz. Microb. Technol. 7: 346-352), an attrition reactor (Ryu and Lee, 1983, Biotechnol. Bioeng. 25: 53-65). Additional reactor types include fluidized bed, upflow blanket, immobilized, and extruder type reactors for hydrolysis and/or fermentation.

In the saccharification step (i.e., hydrolysis step), the cellulosic and/or starch-containing material, e.g., pretreated, is hydrolyzed to break down cellulose, hemicellulose, and/or starch to fermentable sugars, such as glucose, cellobiose, xylose, xylulose, arabinose, mannose, galactose, and/or soluble oligosaccharides. The hydrolysis is performed enzymatically e.g., by a cellulolytic enzyme composition. The enzymes of the compositions can be added simultaneously or sequentially.

Enzymatic hydrolysis may be carried out in a suitable aqueous environment under conditions that can be readily determined by one skilled in the art. In one embodiment, hydrolysis is performed under conditions suitable for the activity of the enzymes(s), i.e., optimal for the enzyme(s). The hydrolysis can be carried out as a fed batch or continuous process where the cellulosic and/or starch-containing material is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art For example, the saccharification can last up to 200 hours, but is typically performed for preferably about 12 to about 120 hours, e.g., about 16 to about 72 hours or about 24 to about 48 hours. The temperature is in the range of preferably about 25° C. to about 70° C., e.g., about 30° C. to about 65° C., about 40° C. to about 60° C., or about 50° C. to about 55° C. The pH is in the range of preferably about 3 to about 8, e.g., about 3.5 to about 7, about 4 to about 6, or about 4.5 to about 5.5. The dry solids content is in the range of preferably about 5 to about 50 wt. %, e.g., about 10 to about 40 wt. % or about 20 to about 30 wt. %.

Saccharification in may be carried out using a cellulolytic enzyme composition. Such enzyme compositions are described below in the “Cellulolytic Enzyme Composition’-section below. The cellulolytic enzyme compositions can comprise any protein useful in degrading the cellulosic-containing material. In one embodiment, the cellulolytic enzyme composition comprises or further comprises one or more (e.g., several) proteins selected from the group consisting of a cellulase, an AA9 (GH61) polypeptide, a hemicellulase, an esterase, an expansin, a ligninolytic enzyme, an oxidoreductase, a pectinase, a protease, and a swollenin.

In another embodiment, the cellulase is preferably one or more (e.g., several) enzymes selected from the group consisting of an endoglucanase, a cellobiohydrolase, and a beta-glucosidase.

In another embodiment, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. In another embodiment, the oxidoreductase is one or more (e.g., several) enzymes selected from the group consisting of a catalase, a laccase, and a peroxidase.

The enzymes or enzyme compositions used in a processes of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

In one embodiment, an effective amount of cellulolytic or hemicellulolytic enzyme composition to the cellulosic-containing material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the cellulosic-containing material.

In one embodiment, such a compound is added at a molar ratio of the compound to glucosyl units of cellulose of about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another embodiment, an effective amount of such a compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc. under conditions as described in WO2012/021401, and the soluble contents thereof. A liquor for cellulolytic enhancement of an AA9 polypeptide (GH61 polypeptide) can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulolytic enzyme preparation. The liquor can be separated from the treated material using a method standard in the art, such as filtration, sedimentation, or centrifugation.

In one embodiment, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In the fermentation step, sugars, released from the cellulosic-containing material, e.g., as a result of the pretreatment and enzymatic hydrolysis steps, are fermented to ethanol, by a host cell or fermenting organism, such as yeast described herein. Hydrolysis (saccharification) and fermentation can be separate or simultaneous.

Any suitable hydrolyzed cellulosic-containing material can be used in the fermentation step in practicing the processes described herein. Such feedstocks include, but are not limited to carbohydrates (e.g., lignocellulose, xylans, cellulose, starch, etc.). The material is generally selected based on economics, i.e., costs per equivalent sugar potential, and recalcitrance to enzymatic conversion.

Production of ethanol by a host cell or fermenting organism using cellulosic-containing material results from the metabolism of sugars (monosaccharides). The sugar composition of the hydrolyzed cellulosic-containing material and the ability of the host cell or fermenting organism to utilize the different sugars has a direct impact in process yields. Prior to Applicant's disclosure herein, strains known in the art utilize glucose efficiently but do not (or very limitedly) metabolize pentoses like xylose, a monosaccharide commonly found in hydrolyzed material.

Compositions of the fermentation media and fermentation conditions depend on the host cell or fermenting organism and can easily be determined by one skilled in the art. Typically, the fermentation takes place under conditions known to be suitable for generating the fermentation product. In some embodiments, the fermentation process is carried out under aerobic or microaerophilic (i.e., where the concentration of oxygen is less than that in air), or anaerobic conditions. In some embodiments, fermentation is conducted under anaerobic conditions (i.e., no detectable oxygen), or less than about 5, about 2.5, or about 1 mmol/L/h oxygen. In the absence of oxygen, the NADH produced in glycolysis cannot be oxidized by oxidative phosphorylation. Under anaerobic conditions, pyruvate or a derivative thereof may be utilized by the host cell as an electron and hydrogen acceptor in order to generate NAD+.

The fermentation process is typically run at a temperature that is optimal for the recombinant fungal cell. For example, in some embodiments, the fermentation process is performed at a temperature in the range of from about 25° C. to about 42° C. Typically the process is carried out a temperature that is less than about 38° C., less than about 35° C., less than about 33° C., or less than about 38° C., but at least about 20° C., 22° C., or 25° C.

A fermentation stimulator can be used in a process described herein to further improve the fermentation, and in particular, the performance of the host cell or fermenting organism, such as, rate enhancement and product yield (e.g., ethanol yield). A “fermentation stimulator” refers to stimulators for growth of the host cells and fermenting organisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

### Cellulolytic Enzymes and Compositions

A cellulolytic enzyme or cellulolytic enzyme composition may be present and/or added during saccharification. A cellulolytic enzyme composition is an enzyme preparation containing one or more (e.g., several) enzymes that hydrolyze cellulosic-containing material. Such enzymes include endoglucanase, cellobiohydrolase, beta-glucosidase, and/or combinations thereof.

In some embodiments, the host cell or fermenting organism comprises one or more (e.g., several) heterologous polynucleotides encoding enzymes that hydrolyze cellulosic-containing material (e.g., an endoglucanase, cellobiohydrolase, beta-glucosidase or combinations thereof). Any enzyme described or referenced herein that hydrolyzes cellulosic-containing material is contemplated for expression in the host cell or fermenting organism.

The cellulolytic enzyme may be any cellulolytic enzyme that is suitable for the host cells and/or the methods described herein (e.g., an endoglucanase, cellobiohydrolase, beta-glucosidase), such as a naturally occurring cellulolytic enzyme or a variant thereof that retains cellulolytic enzyme activity.

In some embodiments, the host cell or fermenting organism comprising a heterologous polynucleotide encoding a cellulolytic enzyme has an increased level of cellulolytic enzyme activity (e.g., increased endoglucanase, cellobiohydrolase, and/or beta-glucosidase) compared to the host cells without the heterologous polynucleotide encoding the cellulolytic enzyme, when cultivated under the same conditions. In some embodiments, the host cell or fermenting organism has an increased level of cellulolytic enzyme activity of at least 5%, e.g., at least 10%, at least 15%, at least 20%, at least 25%, at least 50%, at least 100%, at least 150%, at least 200%, at least 300%, or at 500% compared to the host cell or fermenting organism without the heterologous polynucleotide encoding the cellulolytic enzyme, when cultivated under the same conditions.

Exemplary cellulolytic enzymes that can be used with the host cells and/or the methods described herein include bacterial, yeast, or filamentous fungal cellulolytic enzymes, e.g., obtained from any of the microorganisms described or referenced herein, as described supra under the sections related to proteases.

The cellulolytic enzyme may be of any origin. In an embodiment the cellulolytic enzyme is derived from a strain of Trichoderma, such as a strain of Trichoderma reesei; a strain of Humicola, such as a strain of Humicola insolens, and/or a strain of Chrysosporium, such as a strain of Chrysosporium lucknowense. In a preferred embodiment the cellulolytic enzyme is derived from a strain of Trichoderma reesei.

The cellulolytic enzyme composition may further comprise one or more of the following polypeptides, such as enzymes: AA9 polypeptide (GH61 polypeptide) having cellulolytic enhancing activity, beta-glucosidase, xylanase, beta-xylosidase, CBH 1, CBH 11, or a mixture of two, three, four, five or six thereof.

The further polypeptide(s) (e.g., AA9 polypeptide) and/or enzyme(s) (e.g., beta-glucosidase, xylanase, beta-xylosidase, CBH I and/or CBH 11 may be foreign to the cellulolytic enzyme composition producing organism (e.g., Trichoderma reesei).

In an embodiment the cellulolytic enzyme composition comprises an AA9 polypeptide having cellulolytic enhancing activity and a beta-glucosidase.

In another embodiment the cellulolytic enzyme composition comprises an AA9 polypeptide having cellulolytic enhancing activity, a beta-glucosidase, and a CBH 1.

In another embodiment the cellulolytic enzyme composition comprises an AA9 polypeptide having cellulolytic enhancing activity, a beta-glucosidase, a CBH I and a CBH 11. Other enzymes, such as endoglucanases, may also be comprised in the cellulolytic enzyme composition.

As mentioned above the cellulolytic enzyme composition may comprise a number of difference polypeptides, including enzymes.

In one embodiment, the cellulolytic enzyme composition is a Trichoderma reesei cellulolytic enzyme composition, further comprising Thermoascus aurantiacus AA9 (GH61A) polypeptide having cellulolytic enhancing activity (e.g., WO2005/074656), and Aspergillus oryzae beta-glucosidase fusion protein (e.g., one disclosed in WO2008/057637, in particular shown as SEQ ID NOs: 59 and 60).

In another embodiment the cellulolytic enzyme composition is a Trichoderma reesei cellulolytic enzyme composition, further comprising Thermoascus aurantiacus AA9 (GH61A) polypeptide having cellulolytic enhancing activity (e.g., SEQ ID NO: 2 in WO2005/074656), and Aspergillus fumigatus beta-glucosidase (e.g., SEQ ID NO: 2 of WO2005/047499).

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, and Aspergillus fumigatus beta-glucosidase (e.g., SEQ ID NO: 2 of WO2005/047499).

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, and Aspergillus fumigatus beta-glucosidase (e.g., SEQ ID NO: 2 of WO2005/047499) or a variant disclosed in WO02012/044915 (hereby incorporated by reference), in particular one comprising one or more such as all of the following substitutions: F100D, S283G, N456E, F512Y.

In an embodiment the cellulolytic enzyme composition is a Trichoderma reesei cellulolytic composition, further comprising an AA9 (GH61A) polypeptide having cellulolytic enhancing activity, in particular the one derived from a strain of Penicillium emersonii (e.g., SEQ ID NO: 2 in WO2011/041397), Aspergillus fumigatus beta-glucosidase (e.g., SEQ ID NO: 2 in WO2005/047499) variant with one or more, in particular all of the following substitutions: F100D, S283G, N456E, F512Y and disclosed in WO2012/044915; Aspergillus fumigatus Cel7A CBH1, e.g., the one disclosed as SEQ ID NO: 6 in WO2011/057140 and Aspergillus fumigatus CBH II, e.g., the one disclosed as SEQ ID NO: 18 in WO2011/057140.

In a preferred embodiment the cellulolytic enzyme composition is a Trichoderma reesei, cellulolytic enzyme composition, further comprising a hemicellulase or hemicellulolytic enzyme composition, such as an Aspergillus fumigatus xylanase and Aspergillus fumigatus beta-xylosidase.

In an embodiment the cellulolytic enzyme composition also comprises a xylanase (e.g., derived from a strain of the genus Aspergillus, in particular Aspergillus aculeatus or Aspergillus fumigatus; or a strain of the genus Talaomyces, in particular Talaromyces leycettanus) and/or a beta-xylosidase (e.g., derived from Aspergillus, in particular Aspergillus fumigatus, or a strain of Talaromyces, in particular Talaromyces emersonii).

In an embodiment the cellulolytic enzyme composition is a Trichoderma reesei cellulolytic enzyme composition, further comprising Thermoascus aurantiacus AA9 (GH61A) polypeptide having cellulolytic enhancing activity (e.g., WO2005/074656), Aspergillus oryzae beta-glucosidase fusion protein (e.g., one disclosed in WO2008/057637, in particular as SEQ ID NOs: 59 and 60), and Aspergillus aculeatus xylanase (e.g., Xyl II in WO94/21785).

In another embodiment the cellulolytic enzyme composition comprises a Trichoderma reesei cellulolytic preparation, further comprising Thermoascus aurantiacus GH61A polypeptide having cellulolytic enhancing activity (e.g., SEQ ID NO: 2 in WO2005/074656), Aspergillus fumigatus beta-glucosidase (e.g., SEQ ID NO: 2 of WO2005/047499) and Aspergillus aculeatus xylanase (Xyl II disclosed in WO94/21785).

In another embodiment the cellulolytic enzyme composition comprises a Trichoderma reesei cellulolytic enzyme composition, further comprising Thermoascus aurantiacus AA9 (GH61A) polypeptide having cellulolytic enhancing activity (e.g., SEQ ID NO: 2 in WO2005/074656), Aspergillus fumigatus beta-glucosidase (e.g., SEQ ID NO: 2 of WO2005/047499) and Aspergillus aculeatus xylanase (e.g., Xyl II disclosed in WO94/21785).

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499) and Aspergillus fumigatus xylanase (e.g., Xyl III in WO2006/078256).

In another embodiment the cellulolytic enzyme composition comprises a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499), Aspergillus fumigatus xylanase (e.g., XylI III in WO2006/078256), and CBH I from Aspergillus fumigatus, in particular Cel7A CBH1 disclosed as SEQ ID NO: 2 in WO02011/057140.

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499), Aspergillus fumigatus xylanase (e.g., Xyl III in WO2006/078256), CBH I from Aspergillus fumigatus, in particular Cel7A CBH1 disclosed as SEQ ID NO: 2 in WO2011/057140, and CBH II derived from Aspergillus fumigatus in particular the one disclosed as SEQ ID NO: 4 in WO2013/028928.

In another embodiment the cellulolytic enzyme composition is a Trichoderma reesei cellulolytic enzyme composition, further comprising Penicillium emersonii AA9 (GH61A) polypeptide having cellulolytic enhancing activity, in particular the one disclosed in WO2011/041397, Aspergillus fumigatus beta-glucosidase (e.g., SEQ ID NO: 2 of WO2005/047499) or variant thereof with one or more, in particular all, of the following substitutions: F100D, S283G, N456E, F512Y; Aspergillus fumigatus xylanase (e.g., Xyl III in WO2006/078256), CBH I from Aspergillus fumigatus, in particular Cel7A CBH I disclosed as SEQ ID NO: 2 in WO2011/057140, and CBH II derived from Aspergillus fumigatus, in particular the one disclosed in WO2013/028928.

In another embodiment the cellulolytic enzyme composition is a Trichoderma reesei cellulolytic enzyme composition comprising the CBH I (GENSEQP Accession No. AZY49536 (WO2012/103293); a CBH II (GENSEQP Accession No. AZY49446 (WO2012/103288); a beta-glucosidase variant (GENSEQP Accession No. AZU67153 (WO2012/44915)), in particular with one or more, in particular all, of the following substitutions: F100D, S283G, N456E, F512Y; and AA9 (GH61 polypeptide) (GENSEQP Accession No. BAL61510 (WO2013/028912)).

In another embodiment the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288); a GH10 xylanase (GENSEQP Accession No. BAK46118 (WO2013/019827)); and a beta-xylosidase (GENSEQP Accession No. AZI04896 (WO2011/057140)).

In another embodiment the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288)); and an AA9 (GH61 polypeptide; GENSEQP Accession No. BAL61510 (WO2013/028912)).

In another embodiment the cellulolytic enzyme composition is a Trichoderma reesei cellulolytic enzyme composition comprising a CBH I (GENSEQP Accession No. AZY49536 (WO2012/103293)); a CBH II (GENSEQP Accession No. AZY49446 (WO2012/103288)), an AA9 (GH61 polypeptide; GENSEQP Accession No. BAL61510 (WO2013/028912)), and a catalase (GENSEQP Accession No. BAC11005 (WO2012/130120)).

In an embodiment the cellulolytic enzyme composition is a Trichoderma reesei cellulolytic enzyme composition comprising a CBH I (GENSEQP Accession No. AZY49446 (WO2012/103288); a CBH II (GENSEQP Accession No. AZY49446 (WO2012/103288)), a beta-glucosidase variant (GENSEQP Accession No. AZU67153 (WO2012/44915)), with one or more, in particular all, of the following substitutions: F100D, S283G, N456E, F512Y; an AA9 (GH61 polypeptide; GENSEQP Accession No. BAL61510 (WO2013/028912)), a GH10 xylanase (GENSEQP Accession No. BAK46118 (WO 2013/019827)), and a beta-xylosidase (GENSEQP Accession No. AZI04896 (WO2011/057140)).

In an embodiment the cellulolytic composition is a Trichoderma reesei cellulolytic enzyme preparation comprising an EG I (Swissprot Accession No. P07981), EG II (EMBL Accession No. M19373), CBH I (supra); CBH II (supra); beta-glucosidase variant (supra) with the following substitutions: F100D, S283G, N456E, F512Y; an AA9 (GH61 polypeptide; supra), GH10 xylanase (supra); and beta-xylosidase (supra).

All cellulolytic enzyme compositions disclosed in WO02013/028928 are also contemplated and hereby incorporated by reference.

The cellulolytic enzyme composition comprises or may further comprise one or more (several) proteins selected from the group consisting of a cellulase, a AA9 (i.e., GH61) polypeptide having cellulolytic enhancing activity, a hemicellulase, an expansin, an esterase, a laccase, a ligninolytic enzyme, a pectinase, a peroxidase, a protease, and a swollenin.

In one embodiment the cellulolytic enzyme composition is a commercial cellulolytic enzyme composition. Examples of commercial cellulolytic enzyme compositions suitable for use in a process of the invention include: CELLIC® CTec (Novozymes A/S), CELLIC® CTec2 (Novozymes A/S), CELLIC® CTec3 (Novozymes A/S), CELLUCLAST™ (Novozymes A/S), SPEZYME™ CP (Genencor Int.), ACCELLERASE™ 1000, ACCELLERASE 1500, ACCELLERASE™ TRIO (DuPont), FILTRASE® NL (DSM); METHAPLUS® S/L 100 (DSM), ROHAMEN™ 7069 W (Röhm GmbH), or ALTERNAFUEL® CMAX3™ (Dyadic International, Inc.). The cellulolytic enzyme composition may be added in an amount effective from about 0.001 to about 5.0 wt. % of solids, e.g., about 0.025 to about 4.0 wt. % of solids or about 0.005 to about 2.0 wt. % of solids.

Additional enzymes, and compositions thereof can be found in WO02011/153516 and WO02016/045569 (the contents of which are incorporated herein).

Additional polynucleotides encoding suitable cellulolytic enzymes may be obtained from microorganisms of any genus, including those readily available within the UniProtKB database.

The cellulolytic enzyme coding sequences can also be used to design nucleic acid probes to identify and clone DNA encoding cellulolytic enzymes from strains of different genera or species, as described supra.

The polynucleotides encoding cellulolytic enzymes may also be identified and obtained from other sources including microorganisms isolated from nature (e.g., soil, composts, water, etc.) or DNA samples obtained directly from natural materials (e.g., soil, composts, water, etc.) as described supra.

Techniques used to isolate or clone polynucleotides encoding cellulolytic enzymes are described supra.

In one embodiment, the cellulolytic enzyme has a mature polypeptide sequence of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any cellulolytic enzyme described or referenced herein (e.g., any endoglucanase, cellobiohydrolase, or beta-glucosidase). In one embodiment, the cellulolytic enzyme ha a mature polypeptide sequence that differs by no more than ten amino acids, e.g., by no more than five amino acids, by no more than four amino acids, by no more than three amino acids, by no more than two amino acids, or by one amino acid from any cellulolytic enzyme described or referenced herein. In one embodiment, the cellulolytic enzyme has a mature polypeptide sequence that comprises or consists of the amino acid sequence of any cellulolytic enzyme described or referenced herein, allelic variant, or a fragment thereof having cellulolytic enzyme activity. In one embodiment, the cellulolytic enzyme has an amino acid substitution, deletion, and/or insertion of one or more (e.g., two, several) amino acids. In some embodiments, the total number of amino acid substitutions, deletions and/or insertions is not more than 10, e.g., not more than 9, 8, 7, 6, 5, 4, 3, 2, or 1.

In some embodiments, the cellulolytic enzyme has at least 20%, e.g., at least 40%, at least 50%, at least 60%, at least 70%, at least 80%, at least 90%, at least 95%, at least 96%, at least 97%, at least 98%, at least 99%, or 100% of the cellulolytic enzyme activity of any cellulolytic enzyme described or referenced herein (e.g., any endoglucanase, cellobiohydrolase, or beta-glucosidase) under the same conditions.

In one embodiment, the cellulolytic enzyme coding sequence hybridizes under at least low stringency conditions, e.g., medium stringency conditions, medium-high stringency conditions, high stringency conditions, or very high stringency conditions with the full-length complementary strand of the coding sequence from any cellulolytic enzyme described or referenced herein (e.g., any endoglucanase, cellobiohydrolase, or beta-glucosidase). In one embodiment, the cellulolytic enzyme coding sequence has at least 65%, e.g., at least 70%, at least 75%, at least 80%, at least 85%, at least 85%, at least 90%, at least 91%, at least 92%, at least 93%, at least 94%, at least 95%, at least 96%, at least 97%, at least 98%, at least 99%, or 100% sequence identity with the coding sequence from any cellulolytic enzyme described or referenced herein.

In one embodiment, the polynucleotide encoding the cellulolytic enzyme comprises the coding sequence of any cellulolytic enzyme described or referenced herein (e.g., any endoglucanase, cellobiohydrolase, or beta-glucosidase). In one embodiment, the polynucleotide encoding the cellulolytic enzyme comprises a subsequence of the coding sequence from any cellulolytic enzyme described or referenced herein, wherein the subsequence encodes a polypeptide having cellulolytic enzyme activity. In one embodiment, the number of nucleotides residues in the subsequence is at least 75%, e.g., at least 80%, 85%, 90%, or 95% of the number of the referenced coding sequence.

The cellulolytic enzyme can also include fused polypeptides or cleavable fusion polypeptides, as described supra.

### Fermentation Products

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g., pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide.

In one embodiment, the fermentation product is an alcohol. The term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603. In one embodiment, the fermentation product is ethanol.

In another embodiment, the fermentation product is an alkane. The alkane may be an unbranched or a branched alkane. The alkane can be, but is not limited to, pentane, hexane, heptane, octane, nonane, decane, undecane, or dodecane.

In another embodiment, the fermentation product is a cycloalkane. The cycloalkane can be, but is not limited to, cyclopentane, cyclohexane, cycloheptane, or cyclooctane.

In another embodiment, the fermentation product is an alkene. The alkene may be an unbranched or a branched alkene. The alkene can be, but is not limited to, pentene, hexene, heptene, or octene.

In another embodiment, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another embodiment, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another embodiment, the fermentation product is isoprene.

In another embodiment, the fermentation product is a ketone. The term “ketone” encompasses a substance that contains one or more ketone moieties. The ketone can be, but is not limited to, acetone.

In another embodiment, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another embodiment, the fermentation product is polyketide.

In some embodiments, the host cell or fermenting organism (or processes thereof), provide higher yield of fermentation product (e.g., ethanol) when compared to the same cell without the L-xylulose reductase (LXR) described herein under the same conditions (e.g., after 40 hours of fermentation). In some embodiments, the host cell or fermenting organism (or process thereof) is capable of higher ethanol production compared to the same cell encoding the L-xylulose reductase (LXR) of SEQ ID NO: 305 under the same conditions (e.g., after 40 hours of fermentation) In some embodiments, the process results in at least 0.25%, such as 0.5%, 0.75%, 1.0%, 1.25%, 1.5%, 1.75%, 2%, 3% or 5% higher yield of the fermentation product (e.g., ethanol).

### Recovery

The fermentation product, e.g., ethanol, can optionally be recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

In some embodiments of the methods, the fermentation product after being recovered is substantially pure. With respect to the methods herein, “substantially pure” intends a recovered preparation that contains no more than 15% impurity, wherein impurity intends compounds other than the fermentation product (e.g., ethanol). In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

Suitable assays to test for the production of ethanol and contaminants, and sugar consumption can be performed using methods known in the art. For example, ethanol product, as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of ethanol in the fermentation broth can also be tested with the culture supernatant. Byproducts and residual sugar in the fermentation medium (e.g., glucose or xylose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

The invention may further be described in the following numbered paragraphs:
Paragraph [1]. A recombinant host cell having an active arabinose fermentation pathway, wherein the cell comprises:

(i) a heterologous polynucleotide encoding an aldose reductase (AR),

(ii) a heterologous polynucleotide encoding a L-arabinitol 4-dehydrogenase (LAD), and

(iii) a heterologous polynucleotide encoding a L-xylulose reductase (LXR);

wherein the L-xylulose reductase (LXR) has a mature polypeptide sequence with at least 60%, e.g., at least 65%, 70%, 75%, 80%, 85%, 90%, 95%, 97%, 98%, 99%, or 100% sequence identity to any one of SEQ ID NOs: 297, 300, 302 and 304.

Paragraph [2]. The recombinant host cell of paragraph [1], wherein the L-xylulose reductase (LXR) has a mature polypeptide sequence that differs by no more than ten amino acids, e.g., by no more than five amino acids, by no more than four amino acids, by no more than three amino acids, by no more than two amino acids, or by one amino acid from any one of SEQ ID NOs: 297, 300, 302 and 304.
Paragraph [3]. The recombinant host cell of paragraph [1] or [2], wherein the L-xylulose reductase (LXR) has a mature polypeptide sequence comprising or consisting of the amino acid sequence of any one of SEQ ID NOs: 297, 300, 302 and 304.
Paragraph [4]. The recombinant host cell of any one of paragraphs [1]-[3], wherein the aldose reductase (AR) has a mature polypeptide sequence with at least 60%, e.g., at least 65%, 70%, 75%, 80%, 85%, 90%, 95%, 97%, 98%, 99%, or 100% sequence identity to any one of SEQ ID NOs: 281-285.
Paragraph [5]. The recombinant host cell of any one of paragraphs [1]-[4], wherein the aldose reductase (AR) has a mature polypeptide sequence that differs by no more than ten amino acids, e.g., by no more than five amino acids, by no more than four amino acids, by no more than three amino acids, by no more than two amino acids, or by one amino acid from any one of SEQ ID NOs: 281-285.
Paragraph [6]. The recombinant host cell of any one of paragraphs [1]-[5], wherein the aldose reductase (AR) has a mature polypeptide sequence comprising or consisting of the amino acid sequence of any one of SEQ ID NOs: 281-285.
Paragraph [7]. The recombinant host cell of any one of paragraphs [1]-[6], wherein the L-arabinitol 4-dehydrogenase (LAD) has a mature polypeptide sequence with at least 60%, e.g., at least 65%, 70%, 75%, 80%, 85%, 90%, 95%, 97%, 98%, 99%, or 100% sequence identity to any one of SEQ ID NOs: 286-296.
Paragraph [8]. The recombinant host cell of any one of paragraphs [1]-[7], wherein the wherein the L-arabinitol 4-dehydrogenase (LAD) has a mature polypeptide sequence differs by no more than ten amino acids, e.g., by no more than five amino acids, by no more than four amino acids, by no more than three amino acids, by no more than two amino acids, or by one amino acid from any one of SEQ ID NOs: 286-296.
Paragraph [9]. The recombinant host cell of any one of paragraphs [1]-[8], wherein the L-arabinitol 4-dehydrogenase (LAD) has a mature polypeptide sequence comprising or consisting of the amino acid sequence of any one of SEQ ID NOs: 286-296.
Paragraph [10]. The recombinant host cell of any one of paragraphs [1]-[9], wherein the cell further comprises one or more heterologous polynucleotides selected from:

(iv) a heterologous polynucleotide encoding a xylitol dehydrogenase (XDH); and

(v) a heterologous polynucleotide encoding a xylulokinase (XK).

Paragraph [11]. The recombinant host cell of paragraph [10], wherein the xylitol dehydrogenase (XDH) has a mature polypeptide sequence with at least 60%, e.g., at least 65%, 70%, 75%, 80%, 85%, 90%, 95%, 97%, 98%, 99%, or 100% sequence identity to SEQ ID NO: 309.
Paragraph [12]. The recombinant host cell of paragraph [10] or [11], wherein the xylitol dehydrogenase (XDH) has a mature polypeptide sequence that differs by no more than ten amino acids, e.g., by no more than five amino acids, by no more than four amino acids, by no more than three amino acids, by no more than two amino acids, or by one amino acid from of SEQ ID NO: 309.
Paragraph [13]. The recombinant host cell of any one of paragraphs [10]-[12], wherein the xylitol dehydrogenase (XDH) has a mature polypeptide sequence comprising or consisting of the amino acid sequence of SEQ ID NO: 309.
Paragraph [14]. The recombinant host cell of any one of paragraphs [10]-[13], wherein the xylulokinase (XK) has a mature polypeptide sequence with at least 60%, e.g., at least 65%, 70%, 75%, 80%, 85%, 90%, 95%, 97%, 98%, 99%, or 100% sequence identity to SEQ ID NO: 75, SEQ ID NO: 310 or SEQ ID NO: 311.
Paragraph [15]. The recombinant host cell of any one of paragraphs [10]-[14], wherein the xylulokinase (XK) has a mature polypeptide sequence that differs by no more than ten amino acids, e.g., by no more than five amino acids, by no more than four amino acids, by no more than three amino acids, by no more than two amino acids, or by one amino acid from of SEQ ID NO: 75, SEQ ID NO: 310 or SEQ ID NO: 311.
Paragraph [16]. The recombinant host cell of any one of paragraphs [10]-[15], wherein the xylulokinase (XK) has a mature polypeptide sequence comprising or consisting of the amino acid sequence of SEQ ID NO: 75, SEQ ID NO: 310 or SEQ ID NO: 311.
Paragraph [17]. The recombinant host cell of any one of paragraphs [1]-[16], wherein the heterologous polynucleotide of (i), (ii), (iii), (iv) and/or (v) is operably linked to a promoter that is foreign to the polynucleotide.
Paragraph [18]. The recombinant host cell of any one of paragraphs [1]-[17], wherein the cell further comprises heterologous polynucleotide encoding a non-phosphorylating NADP-dependent glyceraldehyde-3-phosphate dehydrogenase (GAPN).
Paragraph [19]. The recombinant host cell of paragraph [18], wherein the heterologous polynucleotide encoding the NADP-dependent glyceraldehyde-3-phosphate dehydrogenase (GAPN) is operably linked to a promoter that is foreign to the polynucleotide.
Paragraph [20]. The recombinant host cell of paragraph [18] or [19], wherein the heterologous polynucleotide encodes a non-phosphorylating NADP-dependent glyceraldehyde-3-phosphate dehydrogenase (GAPN) having a mature polypeptide sequence with at least 60%, e.g., at least 65%, 70%, 75%, 80%, 85%, 90%, 95%, 97%, 98%, 99%, or 100% sequence identity to any one of SEQ ID NOs: 262-280.
Paragraph [21]. The recombinant host cell of any one of paragraphs [18]-[20], wherein the heterologous polynucleotide encodes a non-phosphorylating NADP-dependent glyceraldehyde-3-phosphate dehydrogenase (GAPN) having a mature polypeptide sequence that differs by no more than ten amino acids, e.g., by no more than five amino acids, by no more than four amino acids, by no more than three amino acids, by no more than two amino acids, or by one amino acid from any one of SEQ ID NOs: 262-280.
Paragraph [22]. The recombinant host cell of any one of paragraphs [18]-[21], wherein the heterologous polynucleotide encodes a non-phosphorylating NADP-dependent glyceraldehyde-3-phosphate dehydrogenase (GAPN) has a mature polypeptide sequence comprising or consisting of the amino acid sequence of any one of SEQ ID NOs: 262-280.
Paragraph [23]. The recombinant host cell of paragraphs [1]-[22], wherein the cell further comprises an active xylose fermentation pathway.
Paragraph [24]. The recombinant host cell of paragraph [23], wherein the cell comprises one or more heterologous polynucleotides selected from:
a heterologous polynucleotide encoding a xylose isomerase (X), and
a heterologous polynucleotide encoding a xylulokinase (XK).
Paragraph [25]. The recombinant host cell of paragraph [23] or [24], wherein the cell comprises one or more heterologous polynucleotides selected from:
a heterologous polynucleotide encoding an aldose reductase (AR),
a heterologous polynucleotide encoding a xylitol dehydrogenase (XDH), and
a heterologous polynucleotide encoding a xylulokinase (XK).
Paragraph [26]. The recombinant host cell of any one of paragraphs [1]-[25], wherein the cell further comprises a heterologous polynucleotide encoding a glucoamylase.
Paragraph [27]. The recombinant host cell of paragraph [26], wherein the glucoamylase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 8, 102-113, 229, 230 and 244-250.
Paragraph [28]. The recombinant host cell of paragraph [26] or [27], wherein the heterologous polynucleotide encoding the glucoamylase is operably linked to a promoter that is foreign to the polynucleotide.
Paragraph [29]. The recombinant host cell of any one of paragraphs [1]-[28], wherein the cell further comprises a heterologous polynucleotide encoding an alpha-amylase.
Paragraph [30]. The recombinant host cell of paragraph [29], wherein the alpha-amylase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 76-101, 121-174, 231 and 251-256.
Paragraph [31]. The recombinant host cell of paragraph [28] or [29], wherein the heterologous polynucleotide encoding the alpha-amylase is operably linked to a promoter that is foreign to the polynucleotide.
Paragraph [32]. The recombinant host cell of any one of paragraphs [1]-[31], wherein the cell further comprises a heterologous polynucleotide encoding a phospholipase.
Paragraph [33]. The recombinant host cell of paragraph [32], wherein the phospholipase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 235, 236, 237, 238, 239, 240, 241 and 242.
Paragraph [34]. The recombinant host cell of paragraph [32] or [33], wherein the heterologous polynucleotide encoding phospholipase is operably linked to a promoter that is foreign to the polynucleotide.
Paragraph [35]. The recombinant host cell of any one of paragraphs [1]-[34], wherein the cell further comprises a heterologous polynucleotide encoding a trehalase.
Paragraph [36]. The recombinant host cell of paragraph [35], wherein the trehalase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 175-226.
Paragraph [37]. The recombinant host cell of paragraph [35] or [36], wherein the heterologous polynucleotide encoding the trehalase is operably linked to a promoter that is foreign to the polynucleotide.
Paragraph [38]. The recombinant host cell of any one of paragraphs [1]-[37], wherein the cell further comprises a heterologous polynucleotide encoding a protease.
Paragraph [39]. The recombinant host cell of paragraph [38], wherein the protease has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 9-73.
Paragraph [40]. The recombinant host cell of paragraph [38] or [39], wherein the heterologous polynucleotide encoding the protease is operably linked to a promoter that is foreign to the polynucleotide.
Paragraph [41]. The recombinant host cell of any one of paragraphs [1]-[40], wherein the cell further comprises a heterologous polynucleotide encoding a pullulanase.
Paragraph [42]. The recombinant host cell of paragraph [41], wherein the pullulanase has a mature polypeptide sequence with at least 60%, e.g., at least 65%, 70%, 75%, 80%, 85%, 90%, 95%, 97%, 98%, 99%, or 100% sequence identity the amino acid sequence of any one of SEQ ID NOs: 114-120.
Paragraph [43]. The recombinant host cell of paragraph [41] or [42], wherein the heterologous polynucleotide encoding the pullulanase is operably linked to a promoter that is foreign to the polynucleotide.
Paragraph [44]. The recombinant host cell of any one of paragraphs [1]-[43], wherein the cell is capable of higher anaerobic growth rate on pentose (e.g., xylose and/or arabinose) compared to the same cell without the heterologous polynucleotide encoding a L-xylulose reductase (LXR) (e.g., under conditions described in Example 2).
Paragraph [45]. The recombinant host cell of any one of paragraphs [1]-[44], wherein the cell is capable of higher anaerobic growth rate on pentose (e.g., xylose and/or arabinose) compared to the same cell encoding the L-xylulose reductase (LXR) of SEQ ID NO: 305 (e.g., under conditions described in Example 2).
Paragraph [46]. The recombinant host cell of any one of paragraphs [1]-[45], wherein the cell is capable of a higher rate of pentose consumption (e.g., at least 5%, 10%, 15%, 20%, 25%, 30%, 35%, 40%, 45%, 50%, 60%, 75% or 90% higher xylose and/or arabinose consumption) compared to the same cell without the heterologous polynucleotide encoding a L-xylulose reductase (LXR) (e.g., under conditions described in Example 2).
Paragraph [47]. The recombinant host cell of any one of paragraphs [1]-[45], wherein the cell is capable of a higher rate of pentose consumption (e.g., at least 5%, 10%, 15%, 20%, 25%, 30%, 35%, 40%, 45%, 50%, 60%, 75% or 90% higher xylose and/or arabinose consumption) compared to the same cell encoding the L-xylulose reductase (LXR) of SEQ ID NO: 305 (e.g., under conditions described in Example 2).
Paragraph [48]. The recombinant host cell of any one of paragraphs [1]-[47], wherein the cell is capable of higher pentose consumption (e.g., xylose and/or arabinose) compared to the same cell without the heterologous polynucleotide encoding a L-xylulose reductase (LXR) at about or after 120 hours fermentation (e.g., under conditions described in Example 2).
Paragraph [49]. The recombinant host cell of any one of paragraphs [1]-[48], wherein the cell is capable of higher pentose consumption (e.g., xylose and/or arabinose) compared to the same cell encoding the L-xylulose reductase (LXR) of SEQ ID NO: 305 at about or after 120 hours fermentation (e.g., under conditions described in Example 2).
Paragraph [50]. The recombinant host cell of paragraph [48] or [49], wherein the cell is capable of consuming more than 65%, e.g., at least 70%, 75%, 80%, 85%, 90%, 95% of pentose (e.g., xylose and/or arabinose) in the medium at about or after 120 hours fermentation (e.g., under conditions described in Example 2).
Paragraph [51]. The recombinant host cell of any one of paragraphs [1]-[50], wherein the cell is capable of higher ethanol production compared to the same cell without the heterologous polynucleotide of (i), (ii), (iii), (iv) and/or (v) under the same conditions (e.g., after 40 hours of fermentation).
Paragraph [52]. The recombinant host cell of any one of paragraphs [1]-[51], wherein the cell is capable of higher ethanol production compared to the same cell encoding the L-xylulose reductase (LXR) of SEQ ID NO: 305 under the same conditions (e.g., after 40 hours of fermentation).
Paragraph [53]. The recombinant host cell of any one of paragraphs [1]-[52], wherein the cell further comprises a heterologous polynucleotide encoding a transketolase (TKL1).
Paragraph [54]. The recombinant host cell of any one of paragraphs [1]-[53], wherein the cell further comprises a heterologous polynucleotide encoding a transaldolase (TAL1).
Paragraph [55]. The recombinant host cell of any one of paragraphs [1]-[54], wherein the cell further comprises a disruption to an endogenous gene encoding a glycerol 3-phosphate dehydrogenase (GPD).
Paragraph [56]. The recombinant host cell of any one of paragraphs [1]-[55], wherein the cell further comprises a disruption to an endogenous gene encoding a glycerol 3-phosphatase (GPP).
Paragraph [57]. The recombinant host cell of paragraph [55] or [56], wherein the GPD and/or GPP gene is inactivated.
Paragraph [58]. The recombinant yeast cell of any of paragraphs [1]-[57], wherein the cell produces a decreased amount of glycerol (e.g., at least 25% less, at least 50% less, at least 60% less, at least 70% less, at least 80% less, or at least 90% less) compared to the cell without the disruption to the endogenous gene encoding the GPD and/or GPP when cultivated under identical conditions.
Paragraph [59]. The recombinant host cell of any one of paragraphs [1]-[58], wherein the cell is a yeast cell.
Paragraph [60]. The recombinant host cell of any one of paragraphs [1]-[59], wherein the cell is a Saccharomyces, Rhodotorula, Schizosaccharomyces, Kuyveromyces, Pichia, 15 Hansenula, Rhodosporidium, Candida, Yarrowia, Lipomyces, Cryptococcus, or Dekkera sp. cell.
Paragraph [61]. The recombinant host cell of any one of paragraphs [1]-[60], wherein the cell is a Saccharomyces cerevisiae cell.
Paragraph [62]. A composition comprising the recombinant host cell of any one of paragraphs [1]-[61] and one or more naturally occurring and/or non-naturally occurring components, such as components are selected from the group consisting of: surfactants, emulsifiers, gums, swelling agents, and antioxidants.
Paragraph [63]. A method of producing a derivative of a recombinant host cell of any one of paragraphs [1]-[61], the method comprising:
(a) providing:
   (i) a first host cell; and
   (ii) a second host cell, wherein the second host cell is a recombinant host cell of any one of paragraphs [1]-[61];
(b) culturing the first host cell and the second host cell under conditions which permit combining of DNA between the first and second host cells;
(c) screening or selecting for a derive host cell.
   Paragraph [64]. A method of producing a fermentation product from a starch-containing or cellulosic-containing material, the method comprising:

(a) saccharifying the starch-containing or cellulosic-containing material; and

(b) fermenting the saccharified material of step (a) with the recombinant host cell of any one of paragraphs [1]-[61] under suitable conditions to produce the fermentation product.

Paragraph [65]. The method of paragraph [64], wherein saccharification of step (a) occurs on a starch-containing material, and wherein the starch-containing material is either gelatinized or ungelatinized starch.
Paragraph [66]. The method of paragraph [65], comprising liquefying the starch-containing material by contacting the material with an alpha-amylase prior to saccharification.
Paragraph [67]. The method of paragraph [65] or [66], wherein liquefying the starch-containing material and/or saccharifying the starch-containing material is conducted in presence of exogenously added protease.
Paragraph [68]. The method of any one of paragraphs [64]-[67], wherein fermentation is performed under reduced nitrogen conditions (e.g., less than 1000 ppm urea or ammonium hydroxide, such as less than 750 ppm, less than 500 ppm, less than 400 ppm, less than 300 ppm, less than 250 ppm, less than 200 ppm, less than 150 ppm, less than 100 ppm, less than 75 ppm, less than 50 ppm, less than 25 ppm, or less than 10 ppm).
Paragraph [69]. The method of any one of paragraphs [64]-[68], wherein fermentation and saccharification are performed simultaneously in a simultaneous saccharification and fermentation (SSF).
Paragraph [70]. The method of any one of paragraphs [64]-[68], wherein fermentation and saccharification are performed sequentially (SHF).
Paragraph [71]. The method of any one of paragraphs paragraph [64]-[70], comprising recovering the fermentation product from the fermentation.
Paragraph [72]. The method of paragraph [71], wherein recovering the fermentation product from the fermentation comprises distillation.
Paragraph [73]. The method of any one of paragraphs [64]-[72], wherein the fermentation product is ethanol.
Paragraph [74]. The method of any one of paragraphs [64]-[72], wherein step (a) comprises contacting the cellulosic and/or starch-containing with an enzyme composition.
Paragraph [75]. The method of any one of paragraphs [64]-[74], wherein saccharification occurs on a cellulosic material, and wherein the cellulosic material is pretreated.
Paragraph [76]. The method of paragraph [75], wherein the pretreatment is a dilute acid pretreatment.
Paragraph [77]. The method of paragraph [75] or [76], wherein saccharification occurs on a cellulosic material, and wherein step (a) comprises contacting the cellulosic enzyme composition, and wherein the enzyme composition comprises one or more enzymes selected from a cellulase, an AA9 polypeptide, a hemicellulase, a CIP, an esterase, an expansin, a ligninolytic enzyme, an oxidoreductase, a pectinase, a protease, and a swollenin.
Paragraph [78]. The method of paragraph [77], wherein the cellulase is one or more enzymes selected from an endoglucanase, a cellobiohydrolase, and a beta-glucosidase.
Paragraph [79]. The method of paragraph [77] or [78], wherein the hemicellulase is one or more enzymes selected a xylanase, an acetylxylan esterase, a feruloyl esterase, an arabinofuranosidase, a xylosidase, and a glucuronidase.
Paragraph [80]. The method of any one of paragraphs [64]-[79], wherein the method results in higher yield of fermentation product when compared to the method using the same cell without the heterologous polynucleotide of (i), (ii), (iii), (iv) and/or (v) under the same conditions.
Paragraph [81]. The method of any one of paragraphs [64]-[80], wherein the method results in higher yield of fermentation product when compared to the method using compared to the same cell encoding the L-xylulose reductase (LXR) of SEQ ID NO: 305 under the same conditions.
Paragraph [82]. The method of paragraph [80] or [81], wherein the method results in at least 0.25% (e.g., 0.5%, 0.75%, 1.0%, 1.25%, 1.5%, 1.75%, 2%, 3% or 5%) higher yield of fermentation product.
Paragraph [83]. The method of any one of paragraphs [64]-[82], wherein fermentation is conducted under low oxygen (e.g., anaerobic) conditions.
Paragraph [84]. The method of any one of paragraphs [64]-[83] wherein a greater amount of pentose (e.g., xylose and/or arabinose) is consumed (e.g., at least 5%, 10%, 15%, 20%, 25%, 30%, 35%, 40%, 45%, 50%, 60%, 75% or 90% more) when compared to the method using the same cell without the heterologous polynucleotide encoding a L-xylulose reductase (LXR) (e.g., under conditions described in Example 2).

Paragraph 85. The method of any one of paragraphs [64]-[84] wherein a greater amount of pentose (e.g., xylose and/or arabinose) is consumed (e.g., at least 5%, 10%, 15%, 20%, 25%, 30%, 35%, 40%, 45%, 50%, 60%, 75% or 90% more) when compared to the method using the same cell encoding the L-xylulose reductase (LXR) of SEQ ID NO: 305 (e.g., under conditions described in Example 2).

Paragraph [86]. The method of any one of any one of paragraphs [64]-[85], wherein more than 65%, e.g., at least 70%, 75%, 80%, 85%, 90%, 95% of pentose (e.g., xylose and/or arabinose) in the medium is consumed (e.g., under conditions described in Example 2).
Paragraph [87]. Use of a recombinant host cell of any one of paragraphs [1]-[61] in the production of ethanol.

The invention described and claimed herein is not to be limited in scope by the specific aspects or embodiments herein disclosed, since these aspects/embodiments are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control. All references are specifically incorporated by reference for that which is described.

The following examples are offered to illustrate certain aspects/embodiments of the present invention, but not in any way intended to limit the scope of the invention as claimed.

### EXAMPLES

### Materials and Methods

Chemicals used as buffers and substrates were commercial products of at least reagent grade.

Yeast strain MBG4779 was prepared according the breeding procedures described in U.S. Pat. No. 8,257,959.

### Example 1: Construction of Yeast Strains Expressing an Active Arabinose Fermentation Pathway

This example describes the construction of yeast cells containing a heterologous L-arabinose utilization pathway. Eight pieces of DNA containing five pathway genes (AR, LAD, LXR, XDH and XK) with a promoter and terminator each were designed to allow for homologous recombination between the eight DNA fragments into the X-3 locus of the yeast MBG4779.

### Construction of the 5′ Homology and Promoter 1 Containing Fragment (Fragment 1)

Synthetic linear uncloned DNA containing 300 bp homology to the X-3 site, S. cerevisiae promoter PGK1 (SEQ ID NO: 4) was synthesized by Thermo Fisher Scientific (Waltham, Mass.) and designated 17ABCK7P.

### Construction of Fragment Containing Aldose Reductase (AR) Coding Sequence (Fragment 2)

Fragment 2 was constructed to contain 50 bp of the 3′ S. cerevisiae promoter PGK1, a aldose reductase (AR) coding sequence, and 50 bp of the 5′ ADH3 terminator. Five DNA fragments were used for fragment 2 each containing a different aldose reductase (Table 8). Two of the XR fragments were synthetic linear uncloned DNA (Twist BioScience, San Francisco, Calif.) and two were originally uncioned synthetic linear but then cloned into a plasmid (GeneArt/Thermo Fisher Scientific, Waltham, Mass.).

The XR from Scheffersomyces stipitis containing fragment was originated as synthetic linear uncloned DNA that was then cloned into a plasmid using a Zero Blunt™ TOPO™ PCR Cloning Kit for Sequencing (Invitrogen/Thermo Fisher, Carlsbad, Calif.). Transformants were selected on 2XYT+Kan agar plates, and plasmid DNA from transformants was sequenced. One clone that had the correct insert by sequencing was designated pMcTs497, which contained the Scheffersomyces stipitis XR gene with a K270R mutation with 50 bp of the 3′ S. cerevisiae promoter PGK1 and 50 bp of the 5′ ADH3 terminator. The K270R mutation was restored to the wild-type with PCR amplification and a synthetic linear DNA containing the K270 codon. The resulting product of 1057 bp contained 50 bp of the 3′ S. cerevisiae promoter PGK1, Scheffersomyces stipitis aldose reductase gene K270 restored (AR), and 50 bp of the 5′ ADH3 terminator. This PCR product was cloned into a plasmid in the Zero Blunt™ TOPO™ PCR Cloning Kit and designated pMcTs498.

Synthetic linear DNAs 16ACZYKP and 16ACZYNP were cloned to a plasmid using the Zero Blunt™ TOPO™ PCR Cloning Kit for sequencing. A sequence confirmed plasmid was designated pMcTs499 and pMcTs500 for 16ACZYKP and 16ACZYNP, respectively.

To generate linear DNA for transformation, plasmids pMcTs498, pMcTs499, pMcTs500 were used as template for PCR. The remaining synthetic linear uncloned DNA was used directly.

TABLE 8
Aldose reductase DNA

| Name | Manufacturer | XR Donor organism | Gene ID | XR SEQ ID | Plasmid |
|---|---|---|---|---|---|
| pMcTs498 | In house | Scheffersomyces stipitis | P31867 | 285 | pMcTs498 |
| 16ACZYKP | GeneArt | Meyerozyma guilliermondii | O94735 | 284 | pMcTs499 |
| 16ACZYNP | GeneArt | Aspergillus niger | A2Q8B5 | 281 | pMcTs500 |
| pSHPs0730B672166SH_H2 | Twist | Magnaporthe oryzae | G4N708 | 283 | N/A |
| pSHPs0730B672166SH_A3 | Twist | Aspergillus oryzae | EFP2C7GS0 | 282 | N/A |

### Construction of Fragment Containing Xylitol Dehydrogenase (XDH) Coding Sequence (Fragment 3)

Fragment 3 was constructed to contain an S. cerevisiae ADH3 terminator, a TDH3 promoter, a xylitol dehydrogenase (XDH) coding sequence from Scheffersomyces stipitis (comprising the coding sequence of SEQ ID NO: 309), a PDC6 terminator and an ADH1 promoter. The DNA sequence containing these features was PCR amplified from a yeast strain that had been previously engineered with these elements from overlapping synthetic uncloned DNA from GeneArt. A first ADH3 terminator and TDH3 promoter fragment was designated 18ABS7OP, a second 50 bp 3′ TDH3 promoter plus Scheffersomyces stipitis gene plus 50 bp 5′ PDC6 terminator fragment was designated 16ACZYUP, and a third PDC terminator and ADH1 promoter fragment was designated 16ACT3QP. These fragments were assembled in a yeast genome using homologous recombination. The fragment containing all these elements listed above was PCR amplified from genomic DNA of the yeast strain.

### Construction of Fragment Containing Xylulokinase (K) Coding Sequence (Fragment 4)

Fragment 4 was constructed to contain a 50 bp sequence of the 3′ S. cerevisiae ADH1 promoter, one of two xylulokinase (XK) coding sequences, a S. cerevisiae TEF1 terminator, and S. cerevisiae PMA1 promoter using methods similar to those described herein (Table 9).

TABLE 9
Xylulokinase DNA

| Name | Manufacturer | XR Donor organism | XK SEQ ID | Gene ID |
|---|---|---|---|---|
| 16ACZYSP | GeneArt | Scheffersomyces stipitis | 310 | A3GF74 |
| 16ACZYPP | GeneArt | Aspergillus niger | 311 | Q8X167 |

### Construction of Fragment Containing L-Xylulose Reductase (LXR) Coding Sequence (Fragment 5)

Fragment 5was constructed to contain one of 12 different L-xylulose reductase coding sequences, each with 50 bp to S. cerevisiae PMA1 promoter at the 5′ end and 50 bp S. cerevisiae STE2 terminator at the 3′ end. Four of the 12 LXRs were originally linear synthetic DNA that was then cloned into a plasmid using the Zero Bunt™ TOPO PCR Cloning Kit for Sequencing. One LXR, 18ABYHTC, was ordered as cloned synthetic DNA. The remaining seven LXRs were linear uncloned synthetic DNA.

TABLE 10
L-xylulose reductase DNA

| Name | Manufacturer | LXR Donor organism | Gene ID | LXR SEQ ID | Plasmid |
|---|---|---|---|---|---|
| 18ABS7LP | GeneArt | Candida arabinofermentans | A0A1E4T269 | 307 | pMcTs501 |
| 18ABS7KP | GeneArt | Ogataea parapolymorpha | E7R859 | 308 | pMcTs502 |
| 17ACWAXP | GeneArt | Trichoderma reesei | Q8NK50 | 305 | pMcTs506 |
| 17ACWAWP | GeneArt | Ambrosiozyma monospora | Q70FD1 | 306 | pMcTs507 |
| 18ABYHTC | GeneArt | Aspergillus niger | G3YG17 | 304 | 18ABYHTC |
| pSHPs0730B672166SH_A2 | Twist | Talaromyces leycettanus | EFP3RCJ9K | 300 | |
| pSHPs0730B672166SH_B2 | Twist | Candida sorboxylosa | EFP5PTNSX | 298 | |
| pSHPs0730B672166SH_C2 | Twist | Aspergillus brasiliensis | EFP7G1BZ1 | 297 | |
| pSHPs0730B672166SH_D2 | Twist | Pichia membranifaciens | EFP16N8ZZ | 303 | |
| pSHPs0730B672166SH_E2 | Twist | Brettanomyces anomalus | EFP480208 | 299 | |
| pSHPs0730B672166SH_F2 | Twist | Kuraishia capsulata | EFP2CH8R5 | 301 | |
| pSHPs0730B672166SH_G2 | Twist | Aspergillus aculeatus | EFP2BNSMN | 302 | |

Linear synthetic DNAs 18ABS7LP, 18ABS7KP, 17ACWAXP and 17ACWAWP were cloned into a plasmid using the Zero Blunt™ TOPO™ PCR Cloning Kit for Sequencing. A sequence-confirmed plasmid for each DNA was identified and designated the name in Table 8. To create linear DNA for transformation plasmids pMcTs501 pMcTs502, pMcTs506, pMcTs507, and 18ABYHTC were used as template in a PCR reaction. The linear synthetic DNA for the other 7 fragments in Table 10 were used directly in the yeast transformations.

### Construction of the STE2 Terminator and TEF2 Promoter Fragment (Fragment 6)

Synthetic cloned DNA containing a S. cerevisiae STE2 terminator and a TEF2 promoter was synthesized by Thermo Fisher Scientific and designated 18ABYHUC. To create linear DNA for transformation plasmid 18ABYHUC was used as template in a PCR reaction to generate the desired 844 bp fragment.

### Construction of Fragment Containing L-Arabinitol 4-Dehydrogenase (LAD) Coding Sequence (Fragment 7)

Fragment 7 was constructed to contain one of 11 different L-arabinitol 4-dehydrogenase coding sequences, each with 50 bp to S. cerevisiae TEF2 promoter at the 5′ end and 50 bp S. cerevisiae PRM9 terminator at the 3′ end. Three of the 12 LXRs were originally linear synthetic DNA that was then cloned into a plasmid using the Zero Blunt™ TOPO™ PCR Cloning Kit for Sequencing. A sequence-confirmed plasmid for each DNA was identified and designated as shown in Table 11. To create linear DNA for transformation, plasmids pMcTs503, pMcTs504, pMcTs505 were used as PCR templates. The linear synthetic DNA for the other eight fragments in Table 9 were used directly in the yeast transformations.

TABLE 11
L-arabinitol 4-dehydrogenase DNA

| Manufacturer | | | | | |
|---|---|---|---|---|---|
| designated name | Manufacturer | LAD Donor organism | Gene ID | LAD SEQ ID | Plasmid |
| 18ABS7RP | GeneArt | Trichoderma reesei | AAE28701 | 287 | pMcTs503 |
| 18ABS7QP | GeneArt | Candida guillermondii | A5DD67 | 288 | pMcTs504 |
| 18ABS7PP | GeneArt | Candida arabinofermentens | A0A1E4SVR6 | 289 | pMcTs505 |
| pSHPs0730B672166SH_A1 | Twist | Penicillium rubens | B6HI95 | 296 | N/A |
| pSHPs0730B672166SH_B1 | Twist | Aspergillus niger | A2QAC0 | 295 | N/A |
| pSHPs0730B672166SH_C1 | Twist | Trichoderma reesei | Q96V44 | 294 | N/A |
| pSHPs0730B672166SH_D1 | Twist | Neurospora crassa | Q7SI09 | 293 | N/A |
| pSHPs0730B672166SH_E1 | Twist | Aspergillus oryzae | Q763T4 | 292 | N/A |
| pSHPs0730B672166SH_F1 | Twist | Talaromyces emersonii | C5J3R8 | 291 | N/A |
| pSHPs0730B672166SH_G1 | Twist | Candida carpophila | EFP5NRTLP | 290 | N/A |
| pSHPs0730B672166SH_H1 | Twist | Meyerozyma caribbica | EFP9CM0L5 | 286 | N/A |

### Construction of the PRM9 Terminator 5′ Homology Containing Fragment (Fragment 8)

Fragment 8 (17ABCLAP) was constructed as a synthetic linear uncloned DNA from Thermo Fisher Scientific containing 300 bp homology to the X-3 site, S. cerevisiae PMR9 terminator and 300 bp homology to the X-3 site.

Integration of the Linear Fragments to Generate a Yeast with Coding Sequences for Heterologous AR, LAD, LAXR, XDH and XK

The yeast MBG4779 was transformed with the fragments described above. In each transformation pool a fixed fragment 1,3,6,8 was used and one of the 12 possible described DNAs of fragment 5 (containing an LXR coding sequence). For fragment 2 a pool of the five ARs was used. For fragment 4 a pool of 2 XKs were used. For fragment 7 a pool of 11 LADs was used. To aid homologous recombination of the left, middle and right fragments at the genomic X-3 sites a plasmid containing Cas9 and guide RNA specific to X-3 (pMcTs442) was also used in the transformation. These four components were transformed into the into S. cerevisiae strain MBG4779 following a yeast electroporation protocol. Transformants were selected on YPD+cloNAT to select for transformants that contain the CRISPR/Cas9 plasmid pMcTs442. Transformants were picked using a Q-pix Colony Picking System (Molecular Devices; San Jose, Calif.) to an OmniTray containing 2% arabinose in YNB where arabinose is the sole carbon source. Strains that were able to grow on these plates after 5-7 days at 30° C. were picked into 2% YPD liquid 96-well plates. The plates were grown for two days then glycerol was added to 20% final concentration and the plates were stored at −80° C. until needed. Integration of five gene cassette was verified by PCR with locus specific primers and subsequent sequencing.

The strains with significant grew on arabinose agar plates described this example are shown in Table 12. Additionally, there were strains that with only slight growth that contained either the A. monospora LXR (Q70FD1) or Kuraishia capsulate LXR (EFP2CH8R5).

TABLE 12

| Strain ID | AR | LAD | LXR | XDH | XK |
|---|---|---|---|---|---|
| S514-C11 | A2Q8B5 | A0A1E4SVR6 | EFP2BNSMN | EFP6RPZ73 | A3GF74 |
| S514-H04 | A2Q8B5 | A0A1E4SVR6 | EFP2BNSMN | EFP6RPZ73 | A3GF74 |
| S514-B06 | A2Q8B5 | A0A1E4SVR6 | EFP2BNSMN | EFP6RPZ73 | Q8X167 |
| S514-C09 | A2Q8B5 | A0A1E4SVR6 | EFP2BNSMN | EFP6RPZ73 | Q8X167 |
| S515-C05 | A2Q8B5 | A0A1E4SVR6 | EFP2BNSMN | EFP6RPZ73 | Q8X167 |
| S515-D05 | A2Q8B5 | A0A1E4SVR6 | EFP2BNSMN | EFP6RPZ73 | Q8X167 |
| S515-H03 | A2Q8B5 | A0A1E4SVR6 | EFP2BNSMN | EFP6RPZ73 | Q8X167 |
| S515-H08 | A2Q8B5 | A0A1E4SVR6 | EFP2BNSMN | EFP6RPZ73 | Q8X167 |
| S516-E01 | A2Q8B5 | A0A1E4SVR6 | EFP2BNSMN | EFP6RPZ73 | Q8X167 |
| S514-D05 | EFP2C7GS0 | A0A1E4SVR6 | EFP2BNSMN | EFP6RPZ73 | A3GF74 |
| S514-C07 | G4N708 | A0A1E4SVR6 | EFP2BNSMN | EFP6RPZ73 | A3GF74 |
| S514-F03 | G4N708 | A0A1E4SVR6 | EFP2BNSMN | EFP6RPZ73 | A3GF74 |
| S515-F10 | G4N708 | A0A1E4SVR6 | EFP2BNSMN | EFP6RPZ73 | A3GF74 |
| S514-E03 | O94735 | A0A1E4SVR6 | EFP2BNSMN | EFP6RPZ73 | A3GF74 |
| S514-E10 | O94735 | A0A1E4SVR6 | EFP2BNSMN | EFP6RPZ73 | A3GF74 |
| S514-H06 | O94735 | A0A1E4SVR6 | EFP2BNSMN | EFP6RPZ73 | A3GF74 |
| S515-B11 | O94735 | A0A1E4SVR6 | EFP2BNSMN | EFP6RPZ73 | A3GF74 |
| S515-C01 | O94735 | A0A1E4SVR6 | EFP2BNSMN | EFP6RPZ73 | A3GF74 |
| S515-G03 | O94735 | A0A1E4SVR6 | EFP2BNSMN | EFP6RPZ73 | A3GF74 |
| S515-G06 | O94735 | A0A1E4SVR6 | EFP2BNSMN | EFP6RPZ73 | A3GF74 |
| S514-C03 | O94735 | A0A1E4SVR6 | EFP2BNSMN | EFP6RPZ73 | Q8X167 |
| S514-H03 | O94735 | A0A1E4SVR6 | EFP2BNSMN | EFP6RPZ73 | Q8X167 |
| S515-E10 | O94735 | A0A1E4SVR6 | EFP2BNSMN | EFP6RPZ73 | Q8X167 |
| S515-F11 | O94735 | A0A1E4SVR6 | EFP2BNSMN | EFP6RPZ73 | Q8X167 |
| S514-F07 | P31867 | A0A1E4SVR6 | EFP2BNSMN | EFP6RPZ73 | A3GF74 |
| S515-H11 | P31867 | A0A1E4SVR6 | EFP2BNSMN | EFP6RPZ73 | A3GF74 |
| S516-D01 | P31867 | A0A1E4SVR6 | EFP2BNSMN | EFP6RPZ73 | A3GF74 |
| S514-G06 | P31867 | A0A1E4SVR6 | EFP2BNSMN | EFP6RPZ73 | Q8X167 |
| S514-G08 | P31867 | A0A1E4SVR6 | EFP2BNSMN | EFP6RPZ73 | Q8X167 |
| S514-H07 | P31867 | A0A1E4SVR6 | EFP2BNSMN | EFP6RPZ73 | Q8X167 |
| S515-D07 | P31867 | A0A1E4SVR6 | EFP2BNSMN | EFP6RPZ73 | Q8X167 |
| S515-D08 | P31867 | A0A1E4SVR6 | EFP2BNSMN | EFP6RPZ73 | Q8X167 |
| S515-E02 | P31867 | A0A1E4SVR6 | EFP2BNSMN | EFP6RPZ73 | Q8X167 |
| S514-G10 | G4N708 | A2QAC0 | EFP2BNSMN | EFP6RPZ73 | A3GF74 |
| S515-A10 | G4N708 | A2QAC0 | EFP2BNSMN | EFP6RPZ73 | A3GF74 |
| S515-A05 | G4N708 | A2QAC0 | EFP2BNSMN | EFP6RPZ73 | Q8X167 |
| S515-H09 | P31867 | A2QAC0 | EFP2BNSMN | EFP6RPZ73 | Q8X167 |
| S516-G01 | O94735 | A5DD67 | EFP2BNSMN | EFP6RPZ73 | A3GF74 |
| S514-H08 | O94735 | A5DD67 | EFP2BNSMN | EFP6RPZ73 | Q8X167 |
| S515-G11 | P31867 | A5DD67 | EFP2BNSMN | EFP6RPZ73 | A3GF74 |
| S514-A04 | O94735 | AAE28701 | EFP2BNSMN | EFP6RPZ73 | A3GF74 |
| S515-F01 | O94735 | AAE28701 | EFP2BNSMN | EFP6RPZ73 | Q8X167 |
| S514-G03 | P31867 | AAE28701 | EFP2BNSMN | EFP6RPZ73 | A3GF74 |
| S514-B08 | A2Q8B5 | B6HI95 | EFP2BNSMN | EFP6RPZ73 | A3GF74 |
| S514-F09 | A2Q8B5 | B6HI95 | EFP2BNSMN | EFP6RPZ73 | A3GF74 |
| S515-B05 | A2Q8B5 | B6HI95 | EFP2BNSMN | EFP6RPZ73 | A3GF74 |
| S515-F09 | EFP2C7GS0 | B6HI95 | EFP2BNSMN | EFP6RPZ73 | A3GF74 |
| S515-E04 | G4N708 | B6HI95 | EFP2BNSMN | EFP6RPZ73 | A3GF74 |
| S516-G03 | O94735 | B6HI95 | EFP2BNSMN | EFP6RPZ73 | A3GF74 |
| S514-G05 | O94735 | B6HI95 | EFP2BNSMN | EFP6RPZ73 | Q8X167 |
| S515-G01 | O94735 | B6HI95 | EFP2BNSMN | EFP6RPZ73 | Q8X167 |
| S515-H07 | P31867 | B6HI95 | EFP2BNSMN | EFP6RPZ73 | A3GF74 |
| S515-A04 | P31867 | B6HI95 | EFP2BNSMN | EFP6RPZ73 | Q8X167 |
| S515-C02 | A2Q8B5 | C5J3R8 | EFP2BNSMN | EFP6RPZ73 | A3GF74 |
| S514-E05 | O94735 | C5J3R8 | EFP2BNSMN | EFP6RPZ73 | Q8X167 |
| S514-G07 | P31867 | C5J3R8 | EFP2BNSMN | EFP6RPZ73 | A3GF74 |
| S515-C11 | P31867 | C5J3R8 | EFP2BNSMN | EFP6RPZ73 | A3GF74 |
| S514-D10 | O94735 | EFP5NRTLP | EFP2BNSMN | EFP6RPZ73 | Q8X167 |
| S514-E04 | EFP2C7GS0 | EFP9CM0L5 | EFP2BNSMN | EFP6RPZ73 | Q8X167 |
| S515-D04 | A2Q8B5 | Q763T4 | EFP2BNSMN | EFP6RPZ73 | A3GF74 |
| S515-E08 | A2Q8B5 | Q763T4 | EFP2BNSMN | EFP6RPZ73 | A3GF74 |
| S516-D03 | A2Q8B5 | Q763T4 | EFP2BNSMN | EFP6RPZ73 | A3GF74 |
| S516-E03 | A2Q8B5 | Q763T4 | EFP2BNSMN | EFP6RPZ73 | A3GF74 |
| S516-H01 | A2Q8B5 | Q763T4 | EFP2BNSMN | EFP6RPZ73 | A3GF74 |
| S514-H11 | A2Q8B5 | Q763T4 | EFP2BNSMN | EFP6RPZ73 | Q8X167 |
| S516-E02 | EFP2C7GS0 | Q763T4 | EFP2BNSMN | EFP6RPZ73 | Q8X167 |
| S515-G04 | G4N708 | Q763T4 | EFP2BNSMN | EFP6RPZ73 | A3GF74 |
| S514-A07 | O94735 | Q763T4 | EFP2BNSMN | EFP6RPZ73 | A3GF74 |
| S515-H10 | O94735 | Q763T4 | EFP2BNSMN | EFP6RPZ73 | Q8X167 |
| S514-B11 | P31867 | Q763T4 | EFP2BNSMN | EFP6RPZ73 | Q8X167 |
| S515-C03 | A2Q8B5 | Q7SI09 | EFP2BNSMN | EFP6RPZ73 | A3GF74 |
| S516-B01 | A2Q8B5 | Q7SI09 | EFP2BNSMN | EFP6RPZ73 | A3GF74 |
| S514-D04 | A2Q8B5 | Q7SI09 | EFP2BNSMN | EFP6RPZ73 | Q8X167 |
| S514-H10 | A2Q8B5 | Q7SI09 | EFP2BNSMN | EFP6RPZ73 | Q8X167 |
| S515-A08 | A2Q8B5 | Q7SI09 | EFP2BNSMN | EFP6RPZ73 | Q8X167 |
| S515-C07 | A2Q8B5 | Q7SI09 | EFP2BNSMN | EFP6RPZ73 | Q8X167 |
| S515-E01 | EFP2C7GS0 | Q7SI09 | EFP2BNSMN | EFP6RPZ73 | Q8X167 |
| S514-F11 | O94735 | Q7SI09 | EFP2BNSMN | EFP6RPZ73 | Q8X167 |
| S514-B04 | P31867 | Q7SI09 | EFP2BNSMN | EFP6RPZ73 | A3GF74 |
| S515-F08 | A2Q8B5 | Q96V44 | EFP2BNSMN | EFP6RPZ73 | A3GF74 |
| S516-G02 | A2Q8B5 | Q96V44 | EFP2BNSMN | EFP6RPZ73 | A3GF74 |
| S514-F08 | G4N708 | Q96V44 | EFP2BNSMN | EFP6RPZ73 | A3GF74 |
| S515-D06 | O94735 | Q96V44 | EFP2BNSMN | EFP6RPZ73 | A3GF74 |
| S515-H01 | O94735 | Q96V44 | EFP2BNSMN | EFP6RPZ73 | A3GF74 |
| S514-A08 | O94735 | Q96V44 | EFP2BNSMN | EFP6RPZ73 | Q8X167 |
| S514-C10 | O94735 | Q96V44 | EFP2BNSMN | EFP6RPZ73 | Q8X167 |
| S512-G04 | A2Q8B5 | A0A1E4SVR6 | EFP3RCJ9K | EFP6RPZ73 | Q8X167 |
| S512-G09 | A2Q8B5 | A0A1E4SVR6 | EFP3RCJ9K | EFP6RPZ73 | Q8X167 |
| S512-H01 | A2Q8B5 | A0A1E4SVR6 | EFP3RCJ9K | EFP6RPZ73 | Q8X167 |
| S512-G02 | O94735 | A0A1E4SVR6 | EFP3RCJ9K | EFP6RPZ73 | A3GF74 |
| S512-F09 | O94735 | A0A1E4SVR6 | EFP3RCJ9K | EFP6RPZ73 | Q8X167 |
| S512-G03 | P31867 | A0A1E4SVR6 | EFP3RCJ9K | EFP6RPZ73 | Q8X167 |
| S512-G01 | O94735 | A2QAC0 | EFP3RCJ9K | EFP6RPZ73 | A3GF74 |
| S512-F04 | P31867 | A2QAC0 | EFP3RCJ9K | EFP6RPZ73 | A3GF74 |
| S512-G05 | P31867 | A2QAC0 | EFP3RCJ9K | EFP6RPZ73 | Q8X167 |
| S512-H03 | P31867 | A2QAC0 | EFP3RCJ9K | EFP6RPZ73 | Q8X167 |
| S511-H03 | A2Q8B5 | B6HI95 | EFP3RCJ9K | EFP6RPZ73 | Q8X167 |
| S512-C01 | O94735 | B6HI95 | EFP3RCJ9K | EFP6RPZ73 | Q8X167 |
| S512-F06 | A2Q8B5 | Q763T4 | EFP3RCJ9K | EFP6RPZ73 | A3GF74 |
| S511-C01 | A2Q8B5 | Q763T4 | EFP3RCJ9K | EFP6RPZ73 | Q8X167 |
| S512-F05 | A2Q8B5 | Q763T4 | EFP3RCJ9K | EFP6RPZ73 | Q8X167 |
| S512-H06 | O94735 | Q7SI09 | EFP3RCJ9K | EFP6RPZ73 | Q8X167 |
| S512-G08 | P31867 | Q7SI09 | EFP3RCJ9K | EFP6RPZ73 | Q8X167 |
| S511-G01 | O94735 | Q96V44 | EFP3RCJ9K | EFP6RPZ73 | A3GF74 |
| S513-F08 | A2Q8B5 | A0A1E4SVR6 | EFP7G1BZ1 | EFP6RPZ73 | A3GF74 |
| S513-H04 | A2Q8B5 | A0A1E4SVR6 | EFP7G1BZ1 | EFP6RPZ73 | A3GF74 |
| S514-C01 | A2Q8B5 | A0A1E4SVR6 | EFP7G1BZ1 | EFP6RPZ73 | Q8X167 |
| S512-G10 | EFP2C7GS0 | A0A1E4SVR6 | EFP7G1BZ1 | EFP6RPZ73 | A3GF74 |
| S513-C11 | EFP2C7GS0 | A0A1E4SVR6 | EFP7G1BZ1 | EFP6RPZ73 | Q8X167 |
| S513-B06 | G4N708 | A0A1E4SVR6 | EFP7G1BZ1 | EFP6RPZ73 | A3GF74 |
| S513-F01 | G4N708 | A0A1E4SVR6 | EFP7G1BZ1 | EFP6RPZ73 | Q8X167 |
| S513-F03 | O94735 | A0A1E4SVR6 | EFP7G1BZ1 | EFP6RPZ73 | A3GF74 |
| S513-G01 | O94735 | A0A1E4SVR6 | EFP7G1BZ1 | EFP6RPZ73 | A3GF74 |
| S513-G02 | O94735 | A0A1E4SVR6 | EFP7G1BZ1 | EFP6RPZ73 | A3GF74 |
| S513-G09 | O94735 | A0A1E4SVR6 | EFP7G1BZ1 | EFP6RPZ73 | A3GF74 |
| S513-H02 | O94735 | A0A1E4SVR6 | EFP7G1BZ1 | EFP6RPZ73 | A3GF74 |
| S513-A01 | O94735 | A0A1E4SVR6 | EFP7G1BZ1 | EFP6RPZ73 | Q8X167 |
| S513-C03 | O94735 | A0A1E4SVR6 | EFP7G1BZ1 | EFP6RPZ73 | Q8X167 |
| S513-D03 | O94735 | A0A1E4SVR6 | EFP7G1BZ1 | EFP6RPZ73 | Q8X167 |
| S514-B01 | O94735 | A0A1E4SVR6 | EFP7G1BZ1 | EFP6RPZ73 | Q8X167 |
| S514-D01 | P31867 | A0A1E4SVR6 | EFP7G1BZ1 | EFP6RPZ73 | A3GF74 |
| S513-G03 | A2Q8B5 | A2QAC0 | EFP7G1BZ1 | EFP6RPZ73 | Q8X167 |
| S513-D02 | O94735 | A2QAC0 | EFP7G1BZ1 | EFP6RPZ73 | A3GF74 |
| S512-C10 | O94735 | A2QAC0 | EFP7G1BZ1 | EFP6RPZ73 | Q8X167 |
| S513-F07 | O94735 | A2QAC0 | EFP7G1BZ1 | EFP6RPZ73 | Q8X167 |
| S513-F09 | O94735 | A2QAC0 | EFP7G1BZ1 | EFP6RPZ73 | Q8X167 |
| S513-F06 | A2Q8B5 | A5DD67 | EFP7G1BZ1 | EFP6RPZ73 | A3GF74 |
| S513-B05 | G4N708 | A5DD67 | EFP7G1BZ1 | EFP6RPZ73 | A3GF74 |
| S513-B07 | O94735 | A5DD67 | EFP7G1BZ1 | EFP6RPZ73 | Q8X167 |
| S513-E01 | O94735 | A5DD67 | EFP7G1BZ1 | EFP6RPZ73 | Q8X167 |
| S513-G06 | A2Q8B5 | AAE28701 | EFP7G1BZ1 | EFP6RPZ73 | A3GF74 |
| S513-A05 | A2Q8B5 | AAE28701 | EFP7G1BZ1 | EFP6RPZ73 | Q8X167 |
| S513-A04 | EFP2C7GS0 | AAE28701 | EFP7G1BZ1 | EFP6RPZ73 | Q8X167 |
| S513-A02 | A2Q8B5 | B6HI95 | EFP7G1BZ1 | EFP6RPZ73 | Q8X167 |
| S513-E06 | G4N708 | B6HI95 | EFP7G1BZ1 | EFP6RPZ73 | Q8X167 |
| S513-A03 | O94735 | B6HI95 | EFP7G1BZ1 | EFP6RPZ73 | Q8X167 |
| S513-C09 | O94735 | B6HI95 | EFP7G1BZ1 | EFP6RPZ73 | Q8X167 |
| S513-E04 | O94735 | B6HI95 | EFP7G1BZ1 | EFP6RPZ73 | Q8X167 |
| S513-F04 | O94735 | B6HI95 | EFP7G1BZ1 | EFP6RPZ73 | Q8X167 |
| S513-D05 | P31867 | B6HI95 | EFP7G1BZ1 | EFP6RPZ73 | A3GF74 |
| S513-C10 | P31867 | B6HI95 | EFP7G1BZ1 | EFP6RPZ73 | Q8X167 |
| S513-D09 | G4N708 | C5J3R8 | EFP7G1BZ1 | EFP6RPZ73 | Q8X167 |
| S513-C02 | O94735 | C5J3R8 | EFP7G1BZ1 | EFP6RPZ73 | A3GF74 |
| S513-G10 | O94735 | C5J3R8 | EFP7G1BZ1 | EFP6RPZ73 | A3GF74 |
| S513-H05 | O94735 | C5J3R8 | EFP7G1BZ1 | EFP6RPZ73 | Q8X167 |
| S514-E01 | O94735 | Q763T4 | EFP7G1BZ1 | EFP6RPZ73 | Q8X167 |
| S513-E11 | P31867 | Q763T4 | EFP7G1BZ1 | EFP6RPZ73 | Q8X167 |
| S513-H11 | P31867 | Q763T4 | EFP7G1BZ1 | EFP6RPZ73 | Q8X167 |
| S513-A11 | G4N708 | Q7SI09 | EFP7G1BZ1 | EFP6RPZ73 | A3GF74 |
| S512-F10 | A2Q8B5 | Q96V44 | EFP7G1BZ1 | EFP6RPZ73 | A3GF74 |
| S513-H06 | A2Q8B5 | Q96V44 | EFP7G1BZ1 | EFP6RPZ73 | Q8X167 |
| S513-B01 | O94735 | Q96V44 | EFP7G1BZ1 | EFP6RPZ73 | Q8X167 |
| S509-C08 | O94735 | A0A1E4SVR6 | G3YG17 | EFP6RPZ73 | A3GF74 |
| S509-A09 | O94735 | A0A1E4SVR6 | G3YG17 | EFP6RPZ73 | Q8X167 |
| S509-E10 | O94735 | A0A1E4SVR6 | G3YG17 | EFP6RPZ73 | Q8X167 |
| S510-H02 | O94735 | A2QAC0 | G3YG17 | EFP6RPZ73 | A3GF74 |
| S509-G08 | A2Q8B5 | B6HI95 | G3YG17 | EFP6RPZ73 | Q8X167 |
| S509-G10 | EFP2C7GS0 | B6HI95 | G3YG17 | EFP6RPZ73 | A3GF74 |
| S509-C06 | P31867 | Q763T4 | G3YG17 | EFP6RPZ73 | A3GF74 |
| S509-H10 | P31867 | Q763T4 | G3YG17 | EFP6RPZ73 | Q8X167 |
| S509-A08 | O94735 | Q7SI09 | G3YG17 | EFP6RPZ73 | A3GF74 |
| S509-D07 | O94735 | Q7SI09 | G3YG17 | EFP6RPZ73 | A3GF74 |
| S509-H09 | O94735 | Q7SI09 | G3YG17 | EFP6RPZ73 | A3GF74 |
| S509-G05 | A2Q8B5 | Q96V44 | G3YG17 | EFP6RPZ73 | A3GF74 |

Table 13 shows a summary of strain growth on arabinose agar plates which was dependent on LXR selection. Those strains with no growth are indicated with (−); stains with minimal growth are indicated with (+/−); stains with the best growth are indicated with (+). Strains with the best growth had expressed an LXR from Aspergillus brasiliensis (SEQ ID NO: 297), Talaromyces leycettanus (SEQ ID NO: 300), Aspergillus aculeatus (SEQ ID NO: 302) or Aspergillus niger (SEQ ID NO: 304), while the strains expressing the remaining LXRs showed only minimal or no growth.

TABLE 13
Arabinose strain growth summary

| | | | Growth agar |
|---|---|---|---|
| LXR | Gene ID | Organism | screening |
| 1 | A0A1E4T269 | C. arabinofermentans | − |
| 2 | E7R859 | O. parapolymorpha | − |
| 3 | Q8NK50 | T. reesei | − |
| 4 | Q70FD1 | A. monospora | +/− |
| 5 | G3YG17 | A. niger | + |
| 6 | EFP3RCJ9K | T. leycettanus | + |
| 7 | EFP5PTNSX | C. sorboxylosa | − |
| 8 | EFP7G1BZ1 | A. brasiliensis | + |
| 9 | EFP16N8ZZ | P. membranifaciens | − |
| 10 | EFP480208 | B. anomalus | − |
| 11 | EFP2CH8R5 | K. capsulata | +/− |
| 12 | EFP2BNSMN | A. aculeatus | + |

### Example 2: Evaluation of Yeast Strains Expressing an Active Arabinose Fermentation Pathway

Yeast strains from Example 1 showing substantial growth (ie those expressing the LXR from, A. brasiliensis, T. leycettanus, A. aculeatus and A. niger) were further evaluated for growth in media where xylose or arabinose were the sole carbon source. The Growth Profiler (Enzyscreen; Heemstede, Netherlands) was used to evaluate strain growth. The Growth Profiler is an incubator that can simultaneously control growth conditions, take images of clear-bottom multi-titer growth plates, and measure cell density over time. A software converts pixels of defined regions per well of each image to RBG (red, blue, green) values; green values are translated to identify growth rates for analysis.

To prepare the strains for evaluation of growth in YNB+2% arabinose or xylose media, yeast strains were grown for 24 hours in YPD medium with 2% glucose, 30° C. and 300 RPM. Yeast cells are centrifuged at 3000 RPM for 10 minutes to pellet the cells. The YPD medium was removed and yeast cells were resuspended in YNB medium. An inoculum of yeast was added to Growth Profiler plates containing 250 uL of medium (YNB with 3% arabinose or 3% xylose). Plates are secured in the Growth Profiler and grown at 250 RPM, 30° C. for 72 hours. Time intervals between each photo was 10 minutes. Growth evaluation was stopped by adding and mixing 50 uL of 8% H₂SO₄. Samples were centrifuged at 3000 RPM for 10 minutes and the supernatant was collected for HPLC analysis for remaining arabinose and xylose concentrations. Slope of each strain was calculated by taking the ratio of rise (green value) over run (time (hours)) during exponential phase. Strains with the highest slopes were able to grow best in the media and those with the least amount of remaining arabinose or xylose consumed the most C5 sugar. Results are shown in Table 14.

TABLE 14

| | Arabinose | | Xylose | |
|---|---|---|---|---|
| | Slope | ARABINOSE | Slope | XYLOSE |
| Strain ID | (G-value/h) | (g/L) | (G-value/h) | (g/L) |
| ER | 0 | 21.50165714 | 0.225084148 | 20.82 |
| S514-C11 | 0.223891 | — | 2.920503 | 8.82 |
| S514-H04 | 0.304674 | 18.2592 | 3.033948 | 7.2 |
| S514-B06 | 0.301397 | 19.971 | 2.950927 | 7.98 |
| S514-C09 | 0.306953 | 20.922 | 2.836496 | 8.16 |
| S515-C05 | 0.26819 | 20.288 | 3.044113 | 9.06 |
| S515-D05 | 0.167696 | 24.9162 | 3.009615 | 8.88 |
| S515-H03 | 0.375604 | 18.8298 | 3.008567 | 10.68 |
| S515-H08 | 0.491814 | 15.9134 | 2.831306 | 8.88 |
| S516-E01 | 0.347358 | 16.9912 | 3.03406 | 10.38 |
| S514-D05 | 0.097607 | 27.0718 | 1.098669 | 12.6 |
| S514-C07 | 0.281654 | 22.19 | 2.387774 | 8.28 |
| S514-F03 | 0.235715 | 24.2822 | 2.581191 | 9.48 |
| S515-F10 | 0.281892 | 20.7952 | 2.834035 | 9.72 |
| S514-E03 | 0.256387 | 22.4436 | 2.04905 | 9.42 |
| S514-E10 | 0.193412 | — | 2.163497 | 10.5 |
| S514-H06 | 0.312106 | 21.6194 | 2.095047 | 8.7 |
| S515-B11 | 0.35427 | 21.4926 | 2.112324 | 10.02 |
| S515-C01 | 0.389105 | 17.3716 | 2.78317 | 8.94 |
| S515-G03 | 0.286616 | 11.2852 | 2.17861 | 10.44 |
| S515-G06 | 0.293631 | 22.5704 | 2.016283 | 10.8 |
| S514-C03 | 0.306685 | 21.239 | 2.08522 | 10.02 |
| S514-H03 | −0.05382 | 29.4176 | 2.144722 | 8.22 |
| S515-E10 | 0.233547 | 23.0142 | 2.192073 | 10.56 |
| S515-F11 | 0.286131 | 21.6194 | 2.146729 | 10.14 |
| S514-F07 | 0.227929 | — | 1.74774 | 9.6 |
| S515-H11 | 0.285432 | 27.1986 | 1.926555 | 10.98 |
| S516-D01 | 0.494728 | 14.9624 | 2.783655 | 11.94 |
| S514-G06 | 0.202736 | 26.6914 | 1.724565 | 9.36 |
| S514-G08 | 0.238605 | 26.6914 | 1.777348 | 9.48 |
| S514-H07 | 0.159451 | 26.945 | 1.798484 | 9.12 |
| S515-D07 | 0.247574 | 26.0574 | 1.768388 | 10.98 |
| S515-D08 | 0.229732 | 26.5646 | 1.744443 | 10.08 |
| S515-E02 | 0.147991 | 26.628 | 1.880161 | 11.88 |
| S514-G10 | 0.312388 | — | 2.767305 | 10.32 |
| S515-A10 | 0.184697 | 27.5156 | 0.918563 | 14.52 |
| S515-A05 | 0.223526 | 26.5012 | 2.475423 | 10.2 |
| S515-H09 | 0.294529 | 23.6482 | 1.800561 | 10.98 |
| S516-G01 | −0.14328 | 29.9882 | 2.137557 | 10.38 |
| S514-H08 | −0.09944 | 29.2908 | 2.111554 | 8.82 |
| S515-G11 | −0.05439 | 29.8614 | 1.731366 | 10.92 |
| S514-A04 | −0.00984 | 23.5214 | 2.882125 | 5.58 |
| S515-F01 | 0.087493 | 22.824 | 2.824389 | 3.3 |
| S514-G03 | 0.014717 | 29.1006 | 1.644223 | 9.96 |
| S514-B08 | 0.478402 | 18.386 | 3.074911 | 7.74 |
| S514-F09 | 0.437208 | — | 2.926999 | 8.52 |
| S515-B05 | 0.523905 | 16.3572 | 3.133496 | 9.24 |
| S515-F09 | 0.412795 | 18.069 | 2.964951 | 9.24 |
| S515-E04 | 0.350307 | 17.1814 | 2.332287 | 8.88 |
| S516-G03 | 0.593214 | 16.2304 | 2.100655 | 10.86 |
| S514-G05 | 0.368751 | 17.6886 | 2.107803 | 8.7 |
| S515-G01 | 0.746399 | 14.5186 | 2.131169 | 10.86 |
| S515-H07 | 0.3148 | 22.1266 | 1.657816 | 10.86 |
| S515-A04 | 0.257812 | 21.8096 | 1.504137 | 12.24 |
| S515-C02 | 0.174764 | 24.1554 | 2.946572 | 9.48 |
| S514-E05 | 0.186016 | 24.726 | 2.081061 | 8.64 |
| S514-G07 | 0.166103 | 26.5012 | 1.734104 | 9.06 |
| S515-C11 | 0.256103 | 27.1986 | 1.477412 | 11.52 |
| S514-D10 | 0.359205 | — | 1.975 | 9.66 |
| S514-E04 | 0.571672 | 8.6224 | 2.89671 | 6.48 |
| S515-D04 | 0.551086 | 14.5186 | 3.005247 | 9 |
| S515-E08 | 0.551203 | 15.4696 | 3.067895 | 9.12 |
| S516-D03 | 0.586343 | 14.1382 | 3.051999 | 9.18 |
| S516-E03 | 0.636337 | 14.899 | 3.102168 | 9.84 |
| S516-H01 | 0.865365 | 13.1238 | 2.995912 | 9.18 |
| S514-H11 | 0.728317 | — | 2.929325 | 9.66 |
| S516-E02 | 0.703683 | 14.6454 | 2.979273 | 9.78 |
| S515-G04 | 1.042094 | 4.5014 | 2.775628 | 4.44 |
| S514-A07 | 0.496838 | 16.0402 | 1.932455 | 8.82 |
| S515-H10 | 0.759813 | 13.6944 | 2.627836 | 10.5 |
| S514-B11 | 0.17521 | | 1.667005 | 11.7 |
| S515-C03 | 0.401786 | 16.0402 | 3.069054 | 9.54 |
| S516-B01 | 0.707663 | 13.0604 | 2.772564 | 10.62 |
| S514-D04 | 0.355574 | — | 2.824911 | 7.92 |
| S514-H10 | 0.604883 | — | 2.973979 | 9.48 |
| S515-A08 | 0.530153 | 15.4062 | 2.90886 | 9.48 |
| S515-C07 | 0.586035 | 14.6454 | 3.150673 | 9.12 |
| S515-E01 | 0.649326 | 15.9768 | 2.743048 | 9.72 |
| S514-F11 | 0.396738 | — | 2.183791 | 9.48 |
| S514-B04 | 0.239392 | 23.2678 | 1.696266 | 9.42 |
| S515-F08 | 0.361978 | 20.8586 | 2.951987 | 9.06 |
| S516-G02 | 0.852543 | 13.0604 | 2.915959 | 9.72 |
| S514-F08 | 0.431982 | 17.5618 | 2.602745 | 7.98 |
| S515-D06 | 1.053344 | 9.1296 | 2.697061 | 9.96 |
| S515-H01 | 0.778018 | 14.8356 | 2.029516 | 10.5 |
| S514-A08 | 0.357256 | 16.5474 | 2.043049 | 8.7 |
| S514-C10 | 0.417053 | — | 1.996473 | 10.74 |
| S512-G04 | 0.443779 | 15.5964 | 3.17579 | 10.92 |
| S512-G09 | 0.406462 | 20.4782 | 2.909094 | 10.68 |
| S512-H01 | 0.541342 | 15.3428 | 3.025252 | 10.86 |
| S512-G02 | −0.01276 | 25.2332 | 2.579761 | 12.96 |
| S512-F09 | 0.384775 | 19.2736 | 2.359821 | 11.94 |
| S512-G03 | 0.283375 | 19.0834 | 1.893099 | 12.96 |
| S512-G01 | 0.793687 | 14.1382 | 2.480385 | 12.84 |
| S512-F04 | 0.481863 | 18.8932 | 2.187117 | 13.32 |
| S512-G05 | 0.345186 | 23.458 | 1.865619 | 12.24 |
| S512-H03 | 0.437481 | 20.1612 | 1.937234 | 13.68 |
| S511-H03 | 0.709499 | 15.85 | 3.287925 | 11.46 |
| S512-C01 | 0.665902 | 17.435 | 2.239868 | 13.08 |
| S512-F06 | 0.757808 | 11.8558 | 2.978545 | 10.92 |
| S511-C01 | 0.725895 | 18.1958 | 3.251609 | 10.14 |
| S512-F05 | 0.669105 | 15.4062 | 3.036416 | 10.38 |
| S512-H06 | 0.699211 | 14.1382 | 2.578299 | 13.08 |
| S512-G08 | 0.469355 | 19.337 | 1.917905 | 12.96 |
| S511-G01 | 0.761954 | 15.9768 | 2.38626 | 11.94 |
| S513-F08 | 0.349422 | 18.4494 | 3.227536 | 8.1 |
| S513-H04 | 0.342447 | 15.9768 | 3.175577 | 7.86 |
| S514-C01 | 0.339727 | 17.118 | 2.902037 | 7.62 |
| S512-G10 | 0.386734 | 14.4552 | 3.027752 | 9.78 |
| S513-C11 | 0.451742 | 13.948 | 2.79311 | 7.44 |
| S513-B06 | 0.279008 | 19.5906 | 2.686594 | 10.26 |
| S513-F01 | 0.349223 | 17.9422 | 2.874943 | 10.32 |
| S513-F03 | 0.322046 | 17.752 | 2.221799 | 10.5 |
| S513-G01 | 0.404345 | 20.4148 | 2.636006 | 10.5 |
| S513-G02 | 0.291819 | 22.6338 | 2.356212 | 9.9 |
| S513-G09 | 0.344041 | 18.703 | 2.328688 | 9.3 |
| S513-H02 | 0.40363 | 17.118 | 2.282683 | 9.48 |
| S513-A01 | 0.405526 | 18.069 | 2.087484 | 18.96 |
| S513-C03 | 0.252879 | 18.1958 | 2.229841 | 9.84 |
| S513-D03 | 0.205545 | 20.0978 | 1.918364 | 10.32 |
| S514-B01 | 0.381516 | 17.0546 | 2.030427 | 8.28 |
| S514-D01 | 0.28958 | 23.3312 | 1.822026 | 8.82 |
| S513-G03 | 0.689008 | 10.3976 | 2.989736 | 7.92 |
| S513-D02 | 0.687881 | 11.412 | 2.211079 | 9.36 |
| S512-C10 | 1.082383 | 4.3112 | 2.934386 | 10.86 |
| S513-F07 | 0.632769 | 11.5388 | 2.080188 | 9.36 |
| S513-F09 | 0.647857 | 12.2362 | 2.14045 | 9.3 |
| S513-F06 | −0.20326 | 28.0228 | 3.111995 | 7.98 |
| S513-B05 | −0.03917 | 28.4666 | 1.629285 | 9.12 |
| S513-B07 | −0.19577 | 28.53 | 2.063536 | 9.3 |
| S513-E01 | −0.1048 | 28.53 | 2.114326 | 11.04 |
| S513-G06 | −0.16182 | 28.847 | 2.728601 | 7.44 |
| S513-A05 | −0.04623 | 28.9738 | 2.935447 | 7.62 |
| S513-A04 | −0.1041 | 29.1006 | 3.04918 | 7.62 |
| S513-A02 | 0.823808 | 8.876 | 3.188441 | 9.66 |
| S513-E06 | 0.56613 | 12.6166 | 2.802197 | 9.12 |
| S513-A03 | 0.710229 | 9.51 | 2.055643 | 9.54 |
| S513-C09 | 0.645218 | 7.925 | 2.129339 | 9.42 |
| S513-E04 | 0.756633 | 10.9682 | 2.090318 | 9.42 |
| S513-F04 | 0.865162 | 9.6368 | 2.503446 | 9.42 |
| S513-D05 | 1.136577 | 5.389 | 3.253874 | 6.66 |
| S513-C10 | 0.267478 | 19.8442 | 1.552885 | 10.38 |
| S513-D09 | 0.160144 | 25.1064 | 2.721037 | 10.14 |
| S513-C02 | 0.268511 | 19.4004 | 2.539023 | 12.06 |
| S513-G10 | 0.176039 | 24.8528 | 2.471286 | 9.06 |
| S513-H05 | 0.204994 | 24.409 | 2.157022 | 9.42 |
| S514-E01 | 0.807711 | 10.0172 | 2.104705 | 8.88 |
| S513-E11 | 0.299618 | 19.7808 | 2.001606 | 10.14 |
| S513-H11 | 0.570877 | 16.2938 | 2.201135 | 9.12 |
| S513-A11 | 0.630132 | 7.4178 | 2.778518 | 8.64 |
| S512-F10 | 0.930637 | 3.9942 | 2.936537 | 10.2 |
| S513-H06 | 0.883345 | 10.3976 | 3.020215 | 7.44 |
| S513-B01 | 0.88628 | 11.9826 | 2.158865 | 10.26 |
| S509-C08 | 0.562551 | 1.7118 | 2.135135 | 10.86 |
| S509-A09 | 0.522801 | 8.7492 | 1.933366 | 11.88 |
| S509-E10 | 0.540302 | 1.7118 | 2.03875 | 10.62 |
| S510-H02 | 0.835795 | 10.9048 | 2.251637 | 11.7 |
| S509-G08 | 0.995938 | 6.8472 | 3.134697 | 9.48 |
| S509-G10 | 0.833948 | 1.3314 | 3.023345 | 8.16 |
| S509-C06 | 0.637888 | 13.2506 | 1.688989 | 11.52 |
| S509-H10 | 0.749673 | 13.3774 | 1.763998 | 12.3 |
| S509-A08 | 0.70811 | 1.0778 | 2.380411 | 10.44 |
| S509-D07 | 0.967568 | 8.0518 | 2.183393 | 10.44 |
| S509-H09 | 0.793033 | 1.1412 | 2.222569 | 10.86 |
| S509-G05 | 0.891624 | 6.4668 | 3.064 | 8.64 |

### Example 3: Construction of Yeast Strains Expressing an Active Arabinose Fermentation Pathway with Heterologous Cones AR, LAD, and LXR

This example describes the construction of yeast cells containing an active arabinose pathway by heterologous expression of only AR, LAD, and LXR. Seven pieces of DNA containing the three heterologous genes (AR, LAD, and LXR) with a promoter and terminator each were designed to allow for homologous recombination between the seven DNA fragments into the XII-5 or XII-2 locus of the yeast MBG4779. The genes for each AR, LAD, and LXR tested in this example have been identified in the examples above to function in yeast strains also expressing XDH and XK. In this example additional copies of XDH and XK were not introduced into the yeast strain.

### Construction of the 5′ Homology and Promoter PGK1 Containing Fragment (Fragment 1)

Two different DNA fragments were designed for fragment 1, one for targeting to the XII-5 site and one for targeting to the XII-2 site. Synthetic linear uncloned DNA containing 379 bp homology to the XII-5 site or 500 bps homology to the XII-2 site and S. cerevisiae promoter PGK1 was synthesized by Thermo Fisher Scientific and designated 18AAQSYW (XII-5) or 18AAQSZW (XII-2). 18AAQSYW (XII-5) was PCR amplified resulting in an 1100 bps linear DNA fragment used for transformation. 18AAQSZW (XII-2) was PCR amplified resulting in a 1200 bp linear DNA fragment used for transformation.

### Construction of Fragment Containing Aldose Reductase (AR) Coding Sequence (Fragment 2)

Fragment 2 was constructed to contain 50 bp of the 3′ S. cerevisiae promoter PGK1, a aldose reductase (AR) coding sequence, and 50 bp of the 5′ ADH3 terminator. Five DNA fragments were used, each containing a different aldose reductase coding sequence. The DNA sources of three of the AR fragments were plasmids constructed as described supra (pMcTS498-500), one uncloned synthetic linear DNA from GeneArt (16ACZYMP), and one genomic DNA (S515-G04). To generate linear DNA for transformation pMcTs498, pMcTs499, pMcTs500, 16ACZYMP, or genomic DNA from S515-G04 were used as a PCR template.

### Construction of ADH3 Terminator and PMA1 Promoter Fragment (Fragment 3)

Fragment 3 was constructed to contain a S. cerevisiae ADH3 terminator and S. cerevisiae PMA1 promoter using methods similar to those described above.

### Construction of Fragment Containing an L-Xylulose Reductase (LXR) Coding Sequence (Fragment 4)

Fragment 4 was constructed to contain a 50 bp 3′ S. cerevisiae PMA1 promoter, a L-xylulose reductase (LXR)_coding sequence, and 50 bp 5′ S. cerevisiae STE2 terminator. Five DNA fragments total were used for fragment 4 each containing a different L-xylulose reductase coding sequence. The DNA sources for three of the LXR genes are genomic DNA from strains constructed supra (S15-G04, S511-B03, and S513-B01), one is plasmid (pMcTs507), and one is cloned synthetic DNA from GeneArt (18ABYHTC). To generate linear DNA for transformation, pMcTs507, 18ABYHTC, or genomic DNA from S15-G04, S511-B03, or S513-B01 were used as PCR template.

### Construction of the STE2 Terminator and TEF2 Promoter Fragment (Fragment 5)

Synthetic cloned DNA containing S. cerevisiae STE2 terminator and TEF2 promoter was synthesized by Thermo Fisher Scientific, designated 18ABYHUC, and used as a PCR template to generate a linear DNA for transformation.

### Construction of Fragment Containing an L-Arabinitol 4-Dehydrogenase (LAD) Coding Sequence (Fragment 6)

Fragment 6 was constructed to contain a 50 bp 3′ S. cerevisiae TEF2 promoter, a L-arabinitol 4-dehydrogenase (LAD) coding sequence, and 50 bp 5′ S. cerevisiae PRM9 terminator. Five DNA fragments total were used for fragment 6 each containing a different L-arabinitol 4-dehydrogenase coding sequence. The DNA sources for four of the LAD genes are genomic DNA from strains constructed supra (S511-B03, S15-G04, S511-A07, and S513-B01) and one is plasmid constructed (pMcTs505). To generate linear DNA for transformation, pMcTs505 or genomic DNA from S511-B03, S15-G04, S511-A07, or S513-B01 were used as a PCR template.

### Construction of the PRM9 Terminator and 3′ Homology Containing Fragment (Fragment 7)

Two different DNA fragments were designed for fragment 7, one for targeting to the XII-5 site and one for targeting to the XII-2 site. Synthetic linear uncloned DNA containing the S. cerevisiae PMR9 terminator and 400 bp homology to the XII-5 site or 481 bps homology to the XII-2 was synthesized by Thermo Fisher Scientific and designated 18ACTCAP (XII-5) or 18AAQS2W (XII-2). 18ACTCAP (XII-5) and 18AAQS2W (XII-2) were PCR amplified resulting in 650 bp and 730 bp linear DNA fragments, respectively.

Integration of the Linear Fragments to Generate a Yeast with Coding Sequences for Heterologous AR, LAD, and LXR

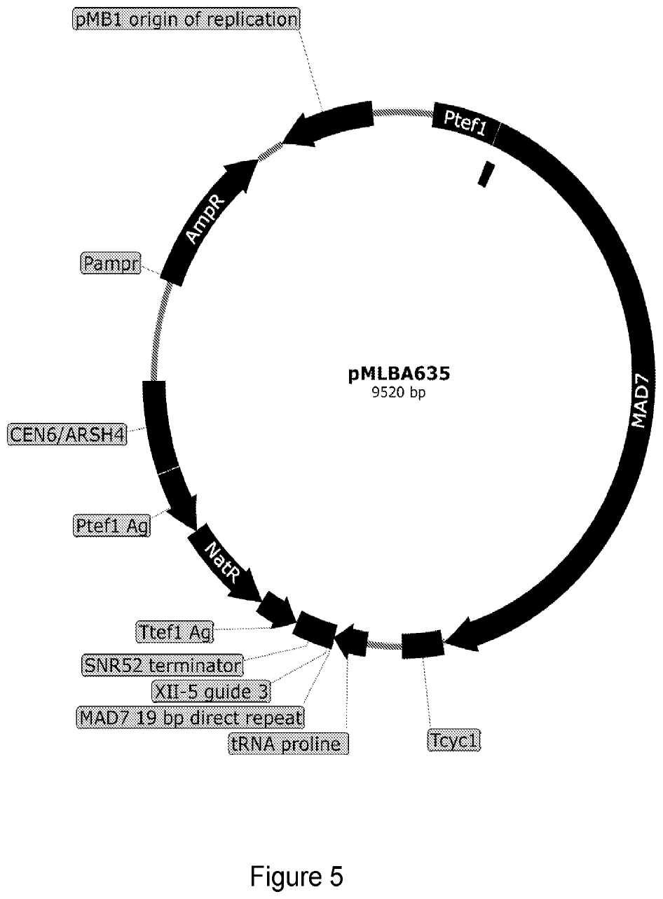

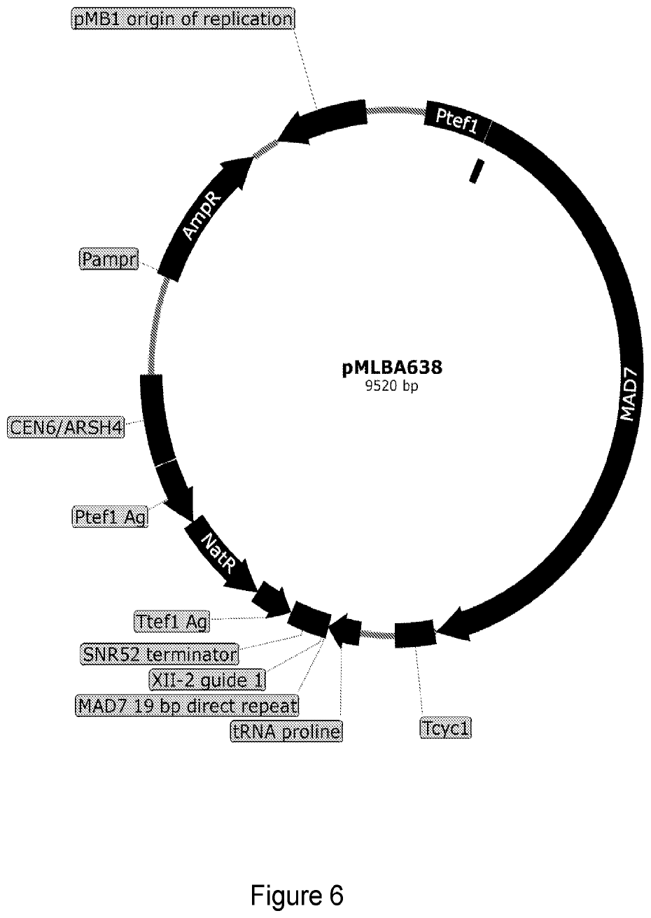

The yeast MBG4779 was transformed with the fragments described above with two different pools of DNA fragments. Pool 1: fragments 1, 3, 5, and 7 were constant, where fragments 1 and 7 used DNA fragments with homology to XII-5. Fragments 2, 4, and 6 were pools of the 5 different coding sequences for AR, LXR, or LAD. Pool 2: fragments 1, 3, 5, and 7 were constant, where fragments 1 and 7 used DNA fragments with homology to XII-2. Fragments 2, 4, and 6 were pools of the 5 different coding sequences for AR, LXR, or LAD. Pool 1 targets the DNA fragments to the XII-5 locus and Pool 2 targets the DNA fragments to the XII-2 locus. To aid homologous recombination of the 7 fragments at the genomic sites XII-5 or XII-2 a plasmid containing the nuclease MAD7 and a guide RNA specific to XII-5 (pMLBA635; FIG. 5) or guide RNA specific to XII-2 (pMLBA638; FIG. 6) was also used in the transformation. Pool 1+pMLBA635 or Pool 2+pMLBA638 were transformed into the into the S. cerevisiae strain MBG4779 following yeast electroporation. Transformants were selected on YPD+cloNAT to select for colonies that contain the CRISPR/MAD7 plasmid pMLBA635 or pMLBA638. 320 transformants targeting to XII-5 or XII-2 were picked using a Q-pix Colony Picking System (Molecular Devices) into YPD (2% glucose)+cloNAT liquid 96-well plates. Plates were grown for 2 days at 30° C., 300 rpm. The Q-pix Colony Picking System (Molecular Devices) system was used to make daughter plates in YPD (2% glucose) liquid 96-well plates. Plates were grown for 3 days at 30° C., 300 rpm. The daughter plates were used to inoculate OmniTrays containing YPD (2% glucose) solid medium. The YPD solid plates were used as a starting culture to inoculate OnmiTrays containing YNB with 2% arabinose as the sole carbon source. 74.2% (475/640) grew on YNB with 2% arabinose as the sole carbon source. Integration of the 3-gene cassette was verified by PCR with locus specific primers and subsequent sequencing. Of the 640 sequenced strains, 243 yielded one gene called for AR, LAD, and LXR in each strain.

In this example 106 of the 125 possible 3-gene expression cassettes were obtained (Table 15), demonstrating growth on YNB+2% arabinose is capable with the only AR, LAD, and LXR.

TABLE 15

| Strain ID | LAD | LXR | AR |
|---|---|---|---|
| S606-A01 | Q763T4 | G3YG17 | O94735 |
| S602-A01 | A2QAC0 | EFP7G1BZ1 | EFP2C7GS0 |
| S603-A01 | Q96V44 | EFP2BNSMN | P31867* |
| S607-A02 | Q7SI09 | Q70FD1 | A2Q8B5 |
| S602-A02 | A2QAC0 | Q70FD1 | A2Q8B5 |
| S606-A02 | A2QAC0 | EFP3RCJ9K | P31867 |
| S605-A03 | Q7SI09 | EFP3RCJ9K | EFP2C7GS0 |
| S603-A03 | Q763T4 | Q70FD1 | A2Q8B5 |
| S603-A05 | Q7SI09 | EFP3RCJ9K | P31867 |
| S601-A05 | A0A1E4SVR6 | G3YG17 | P31867 |
| S604-A05 | Q96V44 | G3YG17 | A2Q8B5 |
| S602-A05 | Q763T4 | Q70FD1 | P31867 |
| S607-A06 | Q763T4 | EFP3RCJ9K | EFP2C7GS0 |
| S605-A06 | Q7SI09 | EFP7G1BZ1 | O94735 |
| S603-A06 | Q763T4 | EFP3RCJ9K | P31867* |
| S603-A07 | Q763T4 | G3YG17 | A2Q8B5 |
| S604-A07 | A2QAC0 | EFP7G1BZ1 | A2Q8B5 |
| S608-A08 | A0A1E4SVR6 | EFP7G1BZ1 | EFP2C7GS0 |
| S607-A09 | A2QAC0 | G3YG17 | EFP2C7GS0 |
| S608-A09 | A2QAC0 | EFP2BNSMN | A2Q8B5 |
| S603-A09 | Q7SI09 | EFP3RCJ9K | O94735 |
| S607-A10 | A0A1E4SVR6 | EFP2BNSMN | O94735 |
| S601-B01 | Q96V44 | EFP2BNSMN | A2Q8B5 |
| S608-B01 | Q96V44 | G3YG17 | O94735 |
| S601-B02 | Q763T4 | EFP7G1BZ1 | EFP2C7GS0 |
| S603-B02 | A2QAC0 | G3YG17 | P31867 |
| S602-B02 | Q96V44 | G3YG17 | EFP2C7GS0 |
| S604-B02 | Q763T4 | Q70FD1 | O94735 |
| S601-B03 | A0A1E4SVR6 | G3YG17 | O94735 |
| S608-B04 | A0A1E4SVR6 | G3YG17 | A2Q8B5 |
| S601-B04 | A0A1E4SVR6 | EFP2BNSMN | EFP2C7GS0 |
| S607-B04 | A0A1E4SVR6 | EFP3RCJ9K | G4N708 |
| S601-B06 | A0A1E4SVR6 | G3YG17 | EFP2C7GS0* |
| S607-B06 | A0A1E4SVR6 | EFP2BNSMN | A2Q8B5 |
| S608-B06 | Q96V44 | EFP7G1BZ1 | EFP2C7GS0 |
| S607-B08 | A2QAC0 | EFP7G1BZ1 | A2Q8B5 |
| S602-B08 | A0A1E4SVR6 | EFP2BNSMN | G4N708 |
| S601-B08 | Q7SI09 | EFP7G1BZ1* | O94735 |
| S607-C01 | Q7SI09 | EFP3RCJ9K | P31867 |
| S602-C01 | A0A1E4SVR6 | EFP3RCJ9K | EFP2C7GS0 |
| S608-C01 | Q763T4 | Q70FD1 | P31867 |
| S601-C02 | A0A1E4SVR6 | G3YG17 | O94735 |
| S601-C03 | Q763T4 | EFP2BNSMN | A2Q8B5 |
| S608-C04 | A2QAC0 | EFP3RCJ9K | P31867 |
| S607-C04 | A2QAC0 | EFP3RCJ9K | EFP2C7GS0* |
| S604-C04 | A2QAC0 | EFP3RCJ9K | O94735 |
| S605-C05 | Q763T4 | EFP2BNSMN | A2Q8B5 |
| S605-C06 | Q763T4 | EFP7G1BZ1 | O94735 |
| S607-C06 | Q763T4 | EFP7G1BZ1 | A2Q8B5 |
| S602-C08 | Q7SI09 | EFP7G1BZ1 | EFP2C7GS0 |
| S605-C08 | Q763T4 | EFP2BNSMN | O94735 |
| S601-C09 | A0A1E4SVR6 | EFP2BNSMN | A2Q8B5 |
| S606-C10 | Q763T4 | EFP2BNSMN | O94735 |
| S602-D01 | A0A1E4SVR6 | EFP3RCJ9K | A2Q8B5 |
| S607-D01 | Q763T4 | EFP7G1BZ1 | EFP2C7GS0* |
| S605-D02 | Q96V44 | EFP2BNSMN | G4N708 |
| S608-D02 | Q96V44 | EFP3RCJ9K | O94735 |
| S603-D02 | Q96V44 | EFP2BNSMN | P31867 |
| S603-D03 | Q763T4 | EFP3RCJ9K | O94735 |
| S601-D03 | A0A1E4SVR6 | EFP7G1BZ1 | G4N708 |
| S605-D03 | Q7SI09 | EFP7G1BZ1 | O94735 |
| S602-D04 | A2QAC0 | G3YG17 | O94735 |
| S603-D04 | A0A1E4SVR6 | Q70FD1 | A2Q8B5 |
| S605-D04 | Q96V44 | G3YG17 | O94735 |
| S603-D05 | Q7SI09 | EFP2BNSMN | EFP2C7GS0* |
| S605-D06 | Q763T4 | G3YG17 | A2Q8B5 |
| S603-D06 | Q763T4 | G3YG17 | O94735 |
| S605-D07 | Q96V44 | EFP7G1BZ1 | A2Q8B5 |
| S607-D07 | A2QAC0 | G3YG17 | A2Q8B5 |
| S603-D07 | Q763T4 | EFP3RCJ9K | P31867 |
| S608-D07 | A2QAC0 | EFP7G1BZ1 | O94735 |
| S602-D07 | Q763T4 | EFP3RCJ9K | P31867 |
| S603-D08 | A2QAC0 | EFP2BNSMN | A2Q8B5 |
| S607-D08 | A0A1E4SVR6 | EFP2BNSMN | EFP2C7GS0 |
| S606-D08 | A0A1E4SVR6 | EFP2BNSMN | P31867 |
| S605-D08 | A0A1E4SVR6 | EFP7G1BZ1 | P31867 |
| S608-D08 | A2QAC0 | G3YG17 | EFP2C7GS0 |
| S607-D09 | A0A1E4SVR6 | G3YG17 | A2Q8B5 |
| S602-D09 | Q7SI09 | G3YG17 | P31867 |
| S606-D10 | Q7SI09 | EFP2BNSMN | P31867 |
| S607-D10 | Q96V44 | EFP7G1BZ1 | P31867 |
| S603-E01 | Q96V44 | G3YG17 | A2Q8B5 |
| S605-E01 | A0A1E4SVR6 | G3YG17 | O94735 |
| S607-E01 | Q96V44 | EFP3RCJ9K | O94735 |
| S603-E02 | A0A1E4SVR6 | EFP7G1BZ1 | O94735 |
| S602-E02 | A0A1E4SVR6 | EFP3RCJ9K | O94735 |
| S604-E02 | A2QAC0* | EFP3RCJ9K | O94735 |
| S601-E02 | Q96V44 | G3YG17 | A2Q8B5 |
| S607-E03 | Q763T4 | EFP7G1BZ1 | O94735 |
| S605-E03 | Q96V44 | EFP2BNSMN | A2Q8B5 |
| S608-E03 | Q763T4 | EFP3RCJ9K | O94735 |
| S603-E04 | Q763T4 | EFP2BNSMN* | EFP2C7GS0 |
| S602-E04 | A0A1E4SVR6 | G3YG17 | O94735 |
| S606-E06 | A2QAC0 | EFP2BNSMN | P31867 |
| S605-E06 | A0A1E4SVR6 | EFP3RCJ9K | A2Q8B5 |
| S605-E07 | Q763T4 | EFP7G1BZ1 | EFP2C7GS0 |
| S606-E07 | A2QAC0 | EFP7G1BZ1 | P31867 |
| S605-E10 | Q7SI09 | G3YG17 | P31867 |
| S607-E10 | Q96V44 | G3YG17 | P31867 |
| S606-E10 | Q7SI09 | EFP2BNSMN | A2Q8B5 |
| S604-F01 | A0A1E4SVR6 | EFP7G1BZ1 | A2Q8B5 |
| S602-F01 | Q763T4 | EFP3RCJ9K | G4N708 |
| S601-F01 | A0A1E4SVR6 | EFP2BNSMN | P31867 |
| S604-F02 | A2QAC0 | G3YG17 | A2Q8B5 |
| S603-F02 | Q763T4 | EFP3RCJ9K | P31867 |
| S602-F02 | Q96V44 | G3YG17 | O94735 |
| S606-F03 | Q7SI09 | EFP7G1BZ1 | G4N708 |
| S607-F04 | Q7SI09 | EFP2BNSMN* | A2Q8B5 |
| S606-F04 | Q7SI09 | EFP7G1BZ1 | P31867 |
| S601-F05 | Q763T4 | G3YG17 | G4N708 |
| S606-F05 | Q96V44 | EFP3RCJ9K | G4N708 |
| S603-F05 | Q96V44 | G3YG17 | P31867 |
| S607-F06 | A0A1E4SVR6 | EFP3RCJ9K | O94735 |
| S606-F07 | A0A1E4SVR6 | EFP3RCJ9K | A2Q8B5 |
| S607-F07 | Q763T4 | EFP3RCJ9K | G4N708 |
| S607-F08 | Q7SI09 | G3YG17 | EFP2C7GS0 |
| S605-F08 | Q7SI09 | G3YG17 | G4N708* |
| S606-F09 | A2QAC0 | EFP2BNSMN | EFP2C7GS0 |
| S601-F09 | Q96V44 | EFP3RCJ9K | P31867 |
| S607-F10 | Q763T4 | EFP2BNSMN | P31867 |
| S603-F10 | Q7SI09 | EFP7G1BZ1 | P31867 |
| S607-G01 | Q96V44 | EFP7G1BZ1 | O94735 |
| S606-G03 | Q763T4 | G3YG17 | EFP2C7GS0 |
| S607-G03 | Q96V44 | EFP2BNSMN | EFP2C7GS0* |
| S601-G03 | Q7SI09 | Q70FD1 | O94735 |
| S605-G03 | Q96V44 | EFP7G1BZ1 | O94735* |
| S602-G03 | A0A1E4SVR6 | EFP3RCJ9K | A2Q8B5 |
| S605-G04 | A2QAC0 | EFP2BNSMN | A2Q8B5* |
| S603-G04 | Q763T4 | EFP3RCJ9K | EFP2C7GS0 |
| S607-G04 | Q763T4 | EFP7G1BZ1 | EFP2C7GS0* |
| S607-G05 | Q96V44 | EFP3RCJ9K | O94735 |
| S607-G06 | Q7SI09 | EFP3RCJ9K | O94735 |
| S602-G06 | A0A1E4SVR6 | Q70FD1 | O94735 |
| S606-G06 | A2QAC0 | EFP3RCJ9K | O94735* |
| S608-G06 | A2QAC0 | G3YG17* | P31867 |
| S601-G06 | A2QAC0 | EFP7G1BZ1 | O94735 |
| S602-G07 | Q96V44 | G3YG17 | P31867 |
| S604-G07 | Q763T4 | Q70FD1 | G4N708 |
| S603-G08 | A2QAC0 | G3YG17 | O94735 |
| S607-G08 | Q7SI09 | G3YG17 | O94735 |
| S608-G08 | A2QAC0 | G3YG17 | G4N708 |
| S601-G08 | Q96V44 | EFP7G1BZ1 | A2Q8B5 |
| S606-G08 | Q7SI09 | EFP3RCJ9K | P31867 |
| S606-G09 | Q96V44 | EFP3RCJ9K | EFP2C7GS0 |
| S607-G09 | Q96V44 | EFP3RCJ9K | P31867 |
| S608-G09 | A2QAC0 | G3YG17 | O94735 |
| S606-G10 | A0A1E4SVR6 | EFP7G1BZ1 | O94735 |
| S605-G10 | Q7SI09 | EFP2BNSMN | O94735 |
| S607-G10 | A0A1E4SVR6 | G3YG17 | G4N708 |
| S608-G10 | Q7SI09 | EFP7G1BZ1 | EFP2C7GS0 |
| S605-H01 | Q96V44 | G3YG17 | O94735 |
| S602-H01 | Q96V44 | Q70FD1 | A2Q8B5 |
| S606-H01 | Q96V44 | EFP7G1BZ1 | EFP2C7GS0 |
| S606-H02 | Q763T4 | EFP3RCJ9K | G4N708 |
| S602-H02 | Q7SI09 | G3YG17 | A2Q8B5 |
| S607-H02 | Q96V44 | G3YG17 | P31867 |
| S605-H03 | Q7SI09 | EFP7G1BZ1 | A2Q8B5 |
| S606-H03 | Q7SI09 | EFP7G1BZ1 | EFP2C7GS0 |
| S604-H04 | Q763T4 | EFP2BNSMN | O94735 |
| S606-H04 | Q763T4 | EFP2BNSMN | A2Q8B5 |
| S607-H04 | Q7SI09 | EFP3RCJ9K | O94735 |
| S601-H04 | Q96V44 | G3YG17 | P31867* |
| S608-H04 | Q96V44 | EFP3RCJ9K | EFP2C7GS0 |
| S603-H04 | Q96V44 | EFP3RCJ9K | O94735 |
| S607-H05 | A0A1E4SVR6 | EFP2BNSMN | P31867 |
| S608-H05 | A0A1E4SVR6 | EFP7G1BZ1 | O94735 |
| S601-H05 | A0A1E4SVR6 | EFP3RCJ9K | EFP2C7GS0* |
| S605-H05 | Q763T4 | G3YG17 | A2Q8B5 |
| S605-H06 | Q7SI09 | G3YG17 | A2Q8B5 |
| S601-H06 | Q763T4 | EFP3RCJ9K | A2Q8B5 |
| S602-H07 | A0A1E4SVR6 | G3YG17* | EFP2C7GS0 |
| S604-H08 | Q7SI09* | EFP3RCJ9K | O94735 |
| S607-H09 | Q763T4 | EFP7G1BZ1 | A2Q8B5 |
| S606-H09 | Q7SI09 | EFP7G1BZ1 | P31867 |
| S607-H10 | A2QAC0 | EFP2BNSMN | EFP2C7GS0 |
| S606-H10 | Q96V44 | G3YG17 | O94735 |

1: A recombinant host cell having an active arabinose fermentation pathway, wherein the cell comprises:
(i) a heterologous polynucleotide encoding an aldose reductase (AR),
(ii) a heterologous polynucleotide encoding a L-arabinitol 4-dehydrogenase (LAD), and
(iii) a heterologous polynucleotide encoding a L-xylulose reductase (LXR);
wherein the L-xylulose reductase (LXR) has a mature polypeptide sequence with at least 80% sequence identity to any one of SEQ ID NOs: 297, 300, 302 and 304.

2: The recombinant host cell of claim 1, wherein the aldose reductase (AR) has a mature polypeptide sequence with at least 80% sequence identity to any one of SEQ ID NOs: 281-285.

3: The recombinant host cell of claim 1, wherein the L-arabinitol 4-dehydrogenase (LAD) has a mature polypeptide sequence with at least 80% sequence identity to any one of SEQ ID NOs: 286-296.

4: The recombinant host cell of claim 1, wherein the cell further comprises one or more heterologous polynucleotides selected from:
(iv) a heterologous polynucleotide encoding a xylitol dehydrogenase (XDH); and
(v) a heterologous polynucleotide encoding a xylulokinase (XK).

5: The recombinant host cell of claim 4, wherein the xylitol dehydrogenase (XDH) has a mature polypeptide sequence with at least 80% sequence identity to SEQ ID NO: 309.

6: The recombinant host cell of claim 4, wherein the xylulokinase (XK) has a mature polypeptide sequence with at least 80% sequence identity to SEQ ID NO: 75, SEQ ID NO: 310 or SEQ ID NO: 311.

7: The recombinant host cell of claim 1, wherein the cell further comprises heterologous polynucleotide encoding a non-phosphorylating NADP-dependent glyceraldehyde-3-phosphate dehydrogenase (GAPN).

8: The recombinant host cell of claim 7, wherein the heterologous polynucleotide encodes a non-phosphorylating NADP-dependent glyceraldehyde-3-phosphate dehydrogenase (GAPN) having a mature polypeptide sequence with at least 80% sequence identity to any one of SEQ ID NOs: 262-280.

9: The recombinant host cell of claim 1, wherein the cell further comprises an active xylose fermentation pathway.

10: The recombinant host cell of claim 9, wherein the cell comprises one or more heterologous polynucleotides selected from:
a heterologous polynucleotide encoding a xylose isomerase (XI), and
a heterologous polynucleotide encoding a xylulokinase (XK).

11: The recombinant host cell of claim 9, wherein the cell comprises one or more heterologous polynucleotides selected from:
a heterologous polynucleotide encoding an aldose reductase (AR),
a heterologous polynucleotide encoding a xylitol dehydrogenase (XDH), and
a heterologous polynucleotide encoding a xylulokinase (XK).

12: The recombinant host cell of claim 1, wherein the cell further comprises a heterologous polynucleotide encoding a glucoamylase.

13: The recombinant host cell of claim 1, wherein the cell further comprises a heterologous polynucleotide encoding an alpha-amylase.

14: The recombinant host cell of claim 1, wherein the cell is capable of higher anaerobic growth rate on xylose compared to the same cell without the heterologous polynucleotide encoding a L-xylulose reductase (LXR).

15: The recombinant host cell of claim 1, wherein the cell is capable of a higher rate of xylose consumption compared to the same cell without the heterologous polynucleotide encoding a L-xylulose reductase (LXR).

16: The recombinant host cell of claim 1, wherein the cell is capable of higher xylose consumption compared to the same cell without the heterologous polynucleotide encoding a L-xylulose reductase (LXR) at 120 hours fermentation.

17: The recombinant yeast cell of claim 1, wherein an endogenous GPD and/or GPP gene is inactivated.

18: The recombinant host cell of claim 1, wherein the cell is a Saccharomyces cerevisiae cell.

19: A method of producing a derivative of a recombinant host cell of claim 1, the method comprising:
(a) providing:
(i) a first host cell; and
(ii) a second host cell, wherein the second host cell is a recombinant host cell of claim 1;
(b) culturing the first host cell and the second host cell under conditions which permit combining of DNA between the first and second host cells;
(c) screening or selecting for a derive host cell.

20: A method of producing a fermentation product from a starch-containing or cellulosic-containing material, the method comprising:
(a) saccharifying the starch-containing or cellulosic-containing material; and
(b) fermenting the saccharified material of step (a) with the recombinant host cell of claim 1 under suitable conditions to produce the fermentation product.
